(19) United States
(12) Patent Application Publication
(10) Pub. No.: US 2004/0019005 A1
(43) Pub. Date: Jan. 29, 2004

(54) Methods for parallel measurement of genetic variations

(76) Inventors: Jeffrey Van Ness, Claremont, CA (US); David J Galas, Claremont, CA (US); Lori K Garrison, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/398,006

(22) PCT Filed: Oct. 1, 2001
(86) PCT No.: PCT/US01/42432

(51) Int. Cl. A61K 48/00; C12Q 1/68

(52) U.S. Cl. 514/44; 435/6

(57) ABSTRACT

The identity of a nucleotide of interest in a target nucleic acid molecule is determined by combining the target with two primers. The first primer is immobilized to a substrate and hybridizes to and extends from a location 3′ of the nucleotide of interest in the target, so as to incorporate the complement of the nucleotide of interest in a first extension product. The second primer then hybridizes to and extends based on the first extension product, which is immobilized to the substrate via the first primer, at a location 3′ of the complement of the nucleotide of interest, so as to incorporate the nucleotide of interest in a second extension product. The second extension product then dissociates from the first extension product and thus from the substrate and re-hybridizes to another first primer molecule that has not extended. The non-extended first primer then extends from a location 3′ of the nucleotide of interest in the second extension product, so as to form, in combination with the second extension product, a double-stranded nucleic acid fragment. The first and second primers are designed to incorporate a portion of the recognition sequence of a restriction endonuclease (RE) that recognizes a partially variable interrupted nucleotide sequence, i.e., a sequence of the form D-N-S where D and S refer to specific nucleotide sequences essential for RE recognition, and N is a sequence consisting of n viable nucleotides also required for RE recognition. The first primer incorporates the sequence D, the second primer incorporates the sequence S, and they are designed, in view of the target, to product a nucleic acid fragment where constant sequences D and S are separated by variable sequence N, where the nucleotide of interest is within region N. Action of the RE on the nucleic acid fragment provides a small nucleic acid fragment that is amendable to characterization, to thereby reveal the identity of the nucleotide of interest.

### BACKGROUND OF THE INVENTION

1. Technical Field

This invention relates to the field of molecular biology, more particularly to methods and compositions involving nucleic acids, and still more particularly to methods and compositions for parallel measurement of genetic variations.

2. Description of the Related Art

The chromosomal mapping and nucleic acid sequencing of each of the 80,000 to 100,000 human genes, achieved through the Human Genome Project, provides an opportunity for a comprehensive approach to the identification of nucleotide loci responsible for genetic diseases. Many of the 150-200 common genetic diseases and ˜600-800 of the rarer genetic diseases are associated with one or more defective genes. Of these, more than 200 human diseases are known to be caused by a defect in a single gene, often resulting in a change of a single amino acid residue. (Olsen, “Biotechnology: An Industry Comes of Age” (National Academic Press, 1986)).

Mutations occurring in somatic cells may induce disease if the mutations affect genes involved in cellular division control, resulting in, for example, tumor formation. In the germline, loss-of-function mutations in many genes can give rise to a detectable phenotype in humans. The number of cell generations in the germline, from one gamete to a gamete in an offspring, may be around 20-fold greater in the male germline than in the female. In the female, an egg is formed after a second meiotic division and lasts for 40 years. Therefore the incidence of different types of germline mutations and chromosomal aberrations depends on the parent of origin.

A majority of mutations, germline or somatic, are of little consequence to the organism since most of the genome appears to lack coding function (about 94%). Even within exon regions there is some tolerance to mutations both due to the degeneracy of the genetic code and because the amino acid substitutions may have only a slight influence on a protein's function. (See, e.g., Strong et al., N. Engl. J. Med. 325:1597 (1991)). With the development of increasingly efficient methods to detect mutations in large DNA segments, the need to predict the functional consequences (e.g., the clinical phenotype) of a mutation become more important.

While point mutations predominate among mutations in the human genome, individual genes may exhibit peculiar patterns of mutations and, accordingly, pose different diagnostic problems. In approximately 60% of cases of Duchenne muscular dystrophy, the mutation involves a deletion of a large segment of the gigantic dystrophine gene. The elucidated mutation causing the fragile X syndrome is characterized by an increased copy number of a particular repeated sequence (CCG)ₙ. Hereditarily unstable DNA of this type may prove to be a more general phenomenon in human disease than is generally recognized.

Molecular genetic techniques have not been employed to a significant extent in the diagnosis of chromosomal aberrations in genetic and malignant disease; cytogenetics remains the preferred technique to investigate these important genetic mechanisms. In an individual with one mutated copy of a tumor suppressor gene, the remaining normal allele may be replaced by a second copy of the mutant allele in one cell per 10³-10⁴. Mechanisms causing this replacement include chromosomal nondisjunction, mitotic recombination, and gene conversion. In contrast, independent mutations destroying the function of the remaining gene copy, are estimated to occur in one cell out of 10⁶.

Sensitive mutation detection techniques offer extraordinary possibilities for mutation screening. For example, analyses may be performed even before the implantation of a fertilized egg. (Holding et al., Lancet 3:532 (1989)). Increasingly efficient genetic tests, may also permit screening for oncogenic mutations in cells exfoliated from the respiratory tract or the bladder in connection with health checkups. (Sidransky et al., Science 252:706, 1991). Alternatively, when an unknown gene causes a genetic disease, methods to monitor DNA sequence variants are useful to study the inheritance of disease through genetic linkage analysis. Notwithstanding these unique applications for the detection of mutations in individual genes, the existing methodology for achieving such applications continues to pose technological and economic challenges. While several different approaches have been pursued, none are sufficiently efficient and cost effective for wide scale application.

Conventional methods for detecting mutations at defined nucleotide loci involve time-consuming linkage analyses in families using limited sets of genetic markers that are difficult to “readout.” Such methods include, e.g., DNA marker haplotyping (that identifies chromosomes with an affected gene) as well as methods for detecting major rearrangements such as large deletions, duplications, translocations and single base pair mutations. These methods include scanning, screening and fluorescence resonance energy transfer (FRET)-based techniques. (See, Cotton, “Mutation Detection” (Oxford University Press, 1997)).

Highly sensitive assays that detect low abundance mutations rely on PCR to amplify the target sequence. Non-selective PCR strategies, however, amplify both mutant and wild-type alleles with approximately equal efficiency. Accordingly, low abundance mutant alleles are represented in only a small fraction of the final product. Thus, if the mutant sequence comprises <25% of the amplified product, it is unlikely that DNA sequencing approaches will be able to detect its presence. Although it is possible to quantify low abundance mutations by first separating the PCR products by cloning and subsequent probing of the clones with allele-specific oligonucleotides (ASOs), this approach is both labor intensive (requiring multiple lengthy procedures) and costly. (Saiki et al., Nature 324:163-166 (1986); Sidransky et al., Science 256:102-105 (1992); and Brennan et al., N. Engl. J. Med. 332:429-435 (1995)).

In contrast to the above, allele-specific PCR methods can rapidly and preferentially amplify mutant alleles. For example, multiple mismatch primers have been used to detect H-ras mutations at a sensitivity of one mutant in 10⁵ wild-type alleles and sensitivity as high as one mutant in 10⁶ wild-type alleles have been reported. (Haliassos et al., Nucleic Acids Res. 17:8093-8099 (1989) and Chen et al., Anal. Biochem 244:191-194 (1997)). These successes are, however, limited to allele-specific primers discriminating through 3′ purine-purine mismatches. For the more common transition mutations, the discriminating mismatch on the 3′ primer end (ie., G:T or C:A mismatch) will be removed in a small fraction of products by polymerase error during extension from the opposite primer on wild-type DNA. Thereafter, these error products are efficiently amplified and generate false positive signals.

It has been suggested that one means to eliminate the polymerase error problem is to deplete wild-type DNA early in the amplification cycles. Several reports have explored selective removal of wild-type DNA by restriction endonuclease digestion in order to enrich for low abundance mutant sequences. These restriction fragment length polymorphism (RFLP) methods detect approximately one mutant in 10⁶ wild-type or better. One approach has employed digestion of genomic DNA followed by PCR amplification of the uncut fragments (RFLP-PCR) to detect very low level mutations within restriction sites in the H-ras and p53 genes. (Sandy et al., Proc. Natl. Acad. Sci. USA 89:890-894 (1992) and Pourzand et al., Mutat. Res. 288:113-121 (1993)). Similar results have been obtained by digestion following PCR and subsequent amplification of the un-cleaved DNA now enriched for mutant alleles (PCR-RFLP). (Kumar et al., Oncogene 3:647-651 (1988); Kumar et al., Oncogene Res. 4:235-241 (1989) and Jacobson et al., Oncogene 9:553-563 (1994)).

Although sensitive and rapid, RFLP detection methods are limited by the requirement that the location of the mutations must coincide with restriction endonuclease recognition sequences. To circumvent this limitation, primers that introduce a restriction site (art of the recognition sequence is in the template DNA) have been employed in “primer-mediated RFLP.” (Jacobson et al., PCR Methods Applicat. 1:299 (1992); Chen et al., Anal. Biochem. 195:51-56 (1991); Di Giuseppe et al., Am. J. Pathol. 144:889-895 (1994); Kahn et al., Oncogene 6:1079-1083 (1991); Levi et al., Cancer Res. 51:3497-3502 (1991) and Mitsudomi et al., Oncogene 6:1353-1362 (1991)). Subsequent investigators have demonstrated, however, that errors are produced at the very next base by polymerase extension from primers having 3′ natural base mismatches. (Hattori et al., Biochem. Biophys. Res. Commun. 202:757-763 (1994); O'Dell et al., Genome Res. 6:558-568 (1996) and Hodanova et al., J. Inherit. Metab. Dis. 20:611-612 (1997)). Such templates fail to cleave during restriction digestion and amplify as false positives that are indistinguishable from true positive products extended from mutant templates.

Use of nucleotide analogs may reduce errors resulting from polymerase extension and improve base conversion fidelity. Nucleotide analogs that are designed to base pair with more than one of the four natural bases are termed “convertides.” Base incorporation opposite different convertides has been tested. (Hoops et al., Nucleic Acids Res. 25:4866-4871 (1997)). For each analog, PCR products were generated using Taq DNA polymerase and primers containing an internal nucleotide analog. The products generated showed a characteristic distribution of the four bases incorporated opposite the analogs.

Due, in part, to the shortcomings in the existing methodology for detecting genetic mutations, there exists an unmet need for rapid and sensitive methods for detecting mutations and parallel measurement of genetic variations. The present invention fulfills this and other related needs by providing methods for parallel measurement of genetic variations that, inter alia, display increased speed, convenience and specificity. As disclosed in detail herein below, methods according to the present invention are based on the incorporation of unique restriction endonuclease restriction sites flanking and/or encompassing genetic variation loci. These methods exploit the high degree of specificity afforded by restriction endonucleases and employ readily available detection techniques.

### SUMMARY OF THE INVENTION

The present invention provides various methods, including those summarized below:

In one aspect, the present invention provides a method for identifying one or more nucleotide(s) at a defined position in a single-stranded target nucleic acid, comprising

(a) providing a first oligonucleotide primer (ODNP) immobilized to a substrate, wherein the first ODNP comprises a nucleotide sequence complementary to a nucleotide sequence of the target nucleic acid at a location 3′ to the defined position, and further comprises a first constant recognition sequence (CRS) of a first strand of an interrupted restriction endonuclease recognition sequence (IRERS), but not a complete IRERS, the complete IRERS being a double-stranded oligonucleotide having the first strand and a second strand and comprising the first and a second CRS linked by a variable recognition sequence (VRS);

(b) exposing the immobilized first ODNP to the target nucleic acid and a second ODNP, wherein the second ODNP comprises a nucleotide sequence complementary to a nucleotide sequence of the complement of the target nucleic acid at a location 3′ to the defined position of the target nucleic acid, and further comprises the second CRS of the second strand of the IRERS;

(c) extending the first and second ODNPs so as to form a fragment having the complete IRERS wherein the nucleotide to be identified is within the VRS of the complete IRERS;

(d) cleaving the fragment with a restriction endonuclease that recognizes the complete IRERS; and

(e) characterizing a product of step (d) to thereby determine the identity of the nucleotide to be identified.

Optionally, the defined position is polymorphic. In certain embodiments, a mutation at the defined position may be associated with a disease. Exemplary diseases include, but are not limited to, bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease. In other embodiments, a mutation at the defined position is associated with drug resistance of a pathogenic microorganism.

In certain embodiments, the single-stranded target nucleic acid is one strand of a denatured double-stranded nucleic acid, including genomic nucleic acid and cDNA. In some embodiments, the single-stranded target nucleic acid is derived from the genome of a pathogenic virus or from the genome or episome of a pathogenic bacterium. In other embodiments, the target nucleic acid is synthetic nucleic acid.

The substrate to which a first ODNP may comprise silicon, glass, paper, ceramic, metal, metalloid, or plastics. A first ODNP may non-covalently immobilized to the substrate. Alternatively, a first ODNP may be covalently immobilized to a substrate at its 5′ terminus. A first immobilized ODNP may be synthesized on the substrate using a technique such as photolithography. Alternatively, a first ODNP may be first synthesized and subsequently immobilized onto the substrate.

Optionally, the nucleotide sequence of the first ODNP that is complementary to the nucleotide sequence of the target nucleic acid is at least 10, 11, 12, 13, or 14 nucleotides in length. Likewise, in certain embodiments, the nucleotide sequence complementary to the nucleotide sequence of the complement of the target nucleic acid in the second ODNP is at least 10, 11, 12, 13 or 14 nucleotides in length.

Optionally, step (c) of the method comprises performing a polymerase chain reaction.

Step (d) of the method may produce a fragment with a blunt end or a 3′ overhang. Alternatively, step (d) produces a fragment with a 5′ overhang, using a restriction enzyme such as EcoN I, wherein optionally the nucleotide to be identified or the complement thereof is within the 5′ overhang, wherein optionally step (e) further comprises filling a 3′ recessed terminus corresponding to the 5′ overhang with one or more nucleoside triphosphates, wherein optionally step (e) further comprises washing the substrate before filling the 3′ recessed terminus, wherein optionally the nucleoside triphosphate comprises a detectable label, wherein optionally the detectable label is a fluorophore or a radioisotope.

The product of step (c) characterized in step (e) may, or may not, be immobilized to the substrate. In certain embodiments, step (e) is performed at least partially by the use of a technique selected from the group consisting of mass spectrometry, liquid chromatography, fluorescence polarization, electron ionization, gel electrophoresis, and capillary electrophoresis.

In another aspect, the invention provides an immoblilized oligonucleotide primer (ODNP), comprising

(a) an oligonucleotide sequence complementary to a nucleotide sequence of a single-stranded target nucleic acid at a location 3′ to a defined position, the oligonucleotide sequence having 3′ and 5′ termini; and

(b) at a location 3′ to the oligonucleotide sequence of (a), a first constant recognition sequence (CRS) of a first strand of an interrupted restriction endonuclease recognition sequence (IRERS), but not a complete IRERS, the complete IRERS being a double-stranded oligonucleotide having the first strand and a second strand and comprising the first CRS and a second CRS linked by a variable recognition sequence (VRS).

In some embodiments, the oligonucleotide sequence of (a) is at least 12, 14, 16, 18, 20, or 22 nucleotides in length. Preferably, the immobilized ODNP further comprises one or more nucleotides complementary to the target nucleic acid at a location 3′ to the first CRS. In certain embodiments, the ODNP is 15-80 nucleotides in length.

The ODNP may be non-covalently immobilized to the substrate. Alternatively, the ODNP is covalently immobilized to the substrate at its 5′ terminus.

In certain embodiments, the complete IRERS is recognizable by EcoN I.

Optionally, the defined position in the target nucleic acid is polymorphic. In certain embodiments, a mutation at the defined position in the target nucleic acid is associated with a disease.

In another aspect, the invention provides an immobilized oligonucleotide primer (ODNP) having regions A, B, C, D, E and F, the ODNP being partially complementary to a target nucleic acid as shown below:

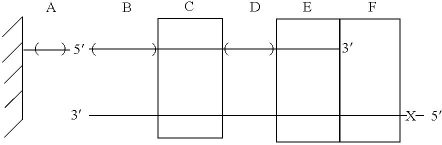

A designates an optional linking element that links the 5′ end of the ODNP to a solid support;

B designates an optional nucleotide sequence;

C designates a nucleotide sequence that is complementary to a nucleotide sequence of a single-stranded target nucleic acid at a location 3′ to a defined position “X” of the target nucleic acid;

D designates a first constant recognition sequence (CRS) of a first strand of an interrupted restriction endonuclease recognition sequence (IRERS), but not a complete IRERS, the complete IRERS being a double-stranded oligonucleotide having the first strand and a second strand and comprising the first CRS and a second CRS linked by a variable recognition sequence (VRS) having a number n of variable nucleotides;

E designates an optional nucleotide sequence; and

F designates an optional gap of nucleotides, where the number of nucleotides within regions E and F is within the range 0 to n-1.

Optionally, in the above-immobilized ODNP, region A is selected from a polyether and a polyester. Region A may be cleavable in some embodiments. Optionally, region B comprises 1 to 50 nucleotides. Optionally, region C comprises 2-30 nucleotides. Optionally, region D comprises 2-6 nucleotides. In a preferred embodiment, region D has the sequence 5′-CCT-3′. Optionally, region E comprises 1-8 nucleotides. Preferably, region E is complementary to the target nucleic acid. Optionally, F comprises 1-8 nucleotides. Optionally, the number of nucleotides with regions B, C, D and E is between 15-80 nucleotides.

The immobilization of the ODNP may be non-covalent attachment to the solid support. Alternatively, the immobilization is covalent attachment to the solid support.

In another aspect of the invention, a composition comprising the immobilized ODNP described above and a target nucleic acid is provided. In certain embodiments, the nucleotide at the defined position in the target nucleic acid may be a single nucleotide polymorphism, polymorphic or a mutation associated with a disease. Exemplary diseases include, but are not limited to, bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

In another aspect, the present invention also provides an array comprising:

(a) a substrate having a plurality of distinct areas; and

(b) a plurality of oligonucleotide primers (ODNPs) immobilized to the distinct areas wherein an ODNP in the plurality comprises

(i) an oligonucleotide sequence complementary to a nucleotide sequence of a single-stranded target nucleic acid at a location 3′ to a defined position at which position a nucleotide is to be identified, the oligonucleotide sequence having 3′ and 5′ termini, and

(ii) at the 3′ terminus of the oligonucleotide sequence of (i), a first constant recognition sequence (CRS) of a first strand of an interrupted restriction endonuclease recognition sequence (IRERS), but not a complete IRERS, the complete IRERS being a double-stranded nucleic acid having the first strand and a second strand and comprising the first CRS and a second CRS linked by a variable recognition sequence (VRS).

In certain embodiments, the ODNPs in any one of the distinct areas of the array are homogeneous, but different from the ODNPs in a second distinct area. In other embodiments, the ODNPs in at least one of the distinct areas are heterogeneous. In some embodiments, the ODNPs in any one of the distinct areas are the same as the ODNPs in a second distinct area.

ODNPs of the array may be non-covalently immobilized to the substrate. Alternatively, they may be covalently immobilized to the substrate at their 5′ termini. In certain embodiments, ODNPs are synthesized on the substrate, for example, using the technology of photolithography. In other embodiments, ODNPs are first synthesized and subsequently immobilized to the substrate.

Optionally, each ODNP is 15-80 nucleotides in length. Preferably, for each ODNP, the oligonucleotide sequence of (i) is at least 10, 11, 12, 13, or 14 nucleotides in length. Preferably, at least one of the ODNPs further comprises one or more nucleotides complementary to the target nucleic acid at a location 3′ to the first CRS.

Optionally, the defined position in the target nucleic acid is polymorphic. Optionally, a mutation at the defined position is associated with a disease.

In certain embodiments, the complete IRERS is recognizable by EcoN I.

In some embodiments, 1000 to 10¹² ODNP molecules of the first set are immobilized in at least one in the plurality of distinct areas.

The substrate of the array may have 2-9, 10-100, 101-400, 401-1000, or more than 1000 distinct areas. It may be made of a material such as silicon, glass, paper, ceramic, metal, metalloid, and plastic. In some embodiments, the surface of the array has raised portions to delineate the distinct areas.

In certain embodiments, the single-stranded target nucleic acid may be one strand of a denatured double-stranded nucleic acid, such as genomic DNA.

Optionally, the target nucleic acids complementary to the ODNP(s) that comprise sequences (i) and (ii) are from one organism. Alternatively, the target nucleic acids complementary to the ODNP(s) that comprise sequences (i) and (ii) are from two or more organisms of one species.

In another aspect, the present invention provides a method, comprising

(a) providing a first set of oligonucleotide primers (ODNPs) immobilized to a substrate in a plurality of distinct areas wherein each ODNP of the first set comprises

(i) an oligonucleotide sequence complementary to a nucleotide sequence of a single-stranded target nucleic acid at a location 3′ to a defined position whereat a nucleotide is to be identified, and

(ii) a first constant recognition sequence (CRS) of a first strand of an interrupted restriction endonuclease recognition sequence (IRERS), but not a complete IRERS, the complete IRERS being a double-stranded nucleic acid having the first strand and a second strand and comprising the first CRS and a second CRS linked by a variable recognition sequence (VRS);

(b) exposing the immobilized first set of ODNPs to one or more target nucleic acids and a second set of ODNPs wherein each ODNP of the second set comprises

(i) an oligonucleotide sequence complementary to a nucleotide sequence of the complement of the single-stranded target nucleic acid at a location 3′ to the defined position, and

(ii) the second CRS of the second strand of the complete IRERS;

(c) extending the ODNPs of the first and second sets so as to form one or more fragments having the complete IRERS wherein the nucleotide(s) to be identified is within the VRS of the complete IRERS;

(d) cleaving the fragment(s) with a restriction endonuclease that recognizes the complete IRERS; and

(e) characterizing a product of step (d) to thereby determine the identity of the nucleotide to be identified.

In certain embodiments, the ODNPs of the first set in any one of the distinct areas are homogeneous, but different from the ODNPs in a second distinct area. In other embodiments, the ODNPs of the first set in at least one of the distinct areas are heterogeneous. In some embodiments, the ODNPs of the first set in any one of the distinct areas are the same as the ODNPs in a second distinct area.

The ODNPs of the first set may be non-covalently immobilized to the substrate. Alternatively, they may be covalently immobilized to the substrate at their 5′ termini. In certain embodiments, the ODNPs are synthesized on the substrate, for example, using the technology of photolithography. In other embodiments, the ODNPs are first synthesized and subsequently immobilized to the substrate.

Optionally, each of the first set of ODNPs is 15-80 nucleotides in length. Optionally, each of the second set of ODNPs is 15-80 nucleotides in length. Preferably, for each ODNP of the first set, the oligonucleotide sequence of (i) is at least 10, 11, 12, 13, or 14 nucleotides in length. Preferably, for each ODNP of the second set, the oligonucleotide sequence of (i) is at least 10, 11, 12, 13, or 14 nucleotides in length. Preferably, at least one of the first set of ODNPs further comprises one or more nucleotides complementary to the target nucleic acid at a location 3′ to the first CRS. Preferably, at least one of the second set of ODNPs further comprises one or more nucleotides complementary to the target nucleic acid at a location 3′ to the second CRS.

Optionally, the defined position in the target nucleic acid is polymorphic. Optionally, a mutation at the defined position is associated with a disease. Exemplary diseases include, but are not limited to, bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

In some embodiments, 1000 to 10¹² ODNP molecules of the first set are immobilized in at least one in the plurality of distinct areas.

The substrate may have 2-9, 10-100, 101-400, 401-1000, or more than 1000 distinct areas. It may be made of a material such as silicon, glass, paper, ceramic, metal, metalloid, plastics and plastic copolymers. In some embodiments, the surface of the array has raised portions to delineate the distinct areas.

In certain embodiments, the single-stranded target nucleic acid may be one strand of a denatured double-stranded nucleic acid, such as genomic DNA, cDNA. In other embodiment the target nucleic acid is synthetic nucleic acid.

Optionally, the target nucleic acids complementary to the ODNP(s) of the first set are from one organism. Alternatively, the target nucleic acids complementary to the ODNP(s) of the first set are from two or more organisms of one species.

Optionally, step (c) of the method comprises performing a polymerase chain reaction.

Step (d) of the method may produce a fragment with a blunt end or a 3′ overhang. Alternatively, step (d) produces a fragment with a 5′ overhang, using a restriction enzyme such as EcoN I, wherein optionally the nucleotide to be identified or the complement thereof is within the 5′ overhang, wherein optionally step (e) further comprises filling a 3′ recessed terminus corresponding to the 5′ overhang with one or more nucleoside triphosphates, wherein optionally the 3′ recessed terminus is filled in with a RNA polymerase, or optionally step (e) further comprises washing the substrate before filling the 3′ recessed terminus, wherein optionally the nucleoside triphosphate comprises a detectable label, wherein optionally the detectable label is a fluorophore or a radioisotope.

The product of step (c) characterized in step (e) may, or may not, be immobilized to the substrate. In certain embodiments, step (e) is performed at least partially by the use of a technique selected from the group consisting of mass spectrometry, liquid chromatography, fluorescence polarization, electron ionization, gel electrophoresis, and capillary electrophoresis.

In another aspect, the invention further provides a method, comprising

(a) exposing the array described above to one or more target nucleic acids and a set of ODNPs wherein each ODNP of the set comprises

(i) an oligonucleotide sequence complementary to a nucleotide sequence of the complement of the single-stranded target nucleic acid at a location 3′ to the defined position, and

(ii) the second CRS of the second strand of the complete IRERS;

(b) extending the immobilized ODNPs of the array and the ODNPs of the set so as to form one or more fragments having the complete IRERS wherein the nucleotide(s) to be identified is within the VRS of the complete IRERS;

(c) cleaving the fragment(s) with a restriction endonuclease that recognizes the complete IRERS; and

(d) characterizing a product of step (d) to thereby determine the identity of the nucleotide to be identified.

In yet another aspect, the invention provides a kit for genotyping comprising the array described above. The kit may further comprise a restriction endonuclease that recognizes a complete IRERS and/or a DNA polymerase.

These and other aspects of the present invention will become evident upon reference to the following detailed description and attached drawings. Each of the references identified herein is incorporated herein by reference. For example, various references as set forth herein describe in more detail certain procedures or compositions (e.g., plasmids, etc.), and are therefore incorporated by reference in their entirety.

### BRIEF DESCRIPTION OF THE SEVERAL VIEWS OF THE DRAWING(S)

FIG. 1 is a chromatogram obtained from electrospray-liquid-chromatography/mass spectrometry-time of flight (ES-LC/MS-TOF) analysis of a 1:10 dilution of a 4-mer ODN (5′-ACGA-3′).

FIG. 2 is a chromatogram obtained from (ES-LC/MS-TOF) analysis of a 1:100 dilution of a 4-mer ODN (5′-ACGA-3′).

FIG. 3 is a chromatogram obtained from (ES-LC/MS-TOF) analysis of a 1:1000 dilution of a 4-mer ODN (5′-ACGA-3′).

FIGS. 4A and 4B are chromatograms obtained from (ES-LC/MS-TOF) analysis of a 1:10 dilution of a 6-mer ODN (5′-ACGATG-3′).

FIG. 5 is a chromatogram obtained from (ES-LC/MS-TOF) analysis of a 1:100 dilution of a 6-mer ODN (5′-ACGATG-3′).

FIG. 6 is a chromatogram obtained from (ES-LC/MS-TOF) analysis of a 1:1000 dilution of 6-mer ODN (5′-ACGATG-3′).

FIGS. 7A and 7B are chromatograms obtained from (ES-LC/MS-TOF) analysis of a 1:10 dilution of a 8-mer ODN (5′-ACGATGCA-3′).

FIG. 8 is a chromatogram obtained from (ES-LC/MS-TOF) analysis of a 1:100 dilution of a 8-mer ODN (5′-ACGATGCA-3′).

FIGS. 9A and 9B are chromatograms obtained from (ES-LC/MS-TOF) analysis of a 1:10 dilution of the 10-mer ODN of SEQ ID NO: 8.

FIG. 10 is a chromatogram obtained from (ES-LC/MS-TOF) analysis of a 1:100 dilution of the 10-mer ODN of SEQ ID NO: 8.

FIG. 11 is a chromatogram obtained from (ES-LC/MS-TOF) analysis of a 1:1000 dilution of the 10-mer ODN of SEQ ID NO: 8.

FIG. 12 is a diagram of major steps in one aspect of the present method for identifying a nucleotide at a defined position in a target nucleic acid using two ODNPs and an exemplary restriction endonuclease recognition sequence for EcoN I.

FIG. 13 is a high pressure liquid chromatogram (HPLC) of a set of 4, 6, 8 and 10 nucleotide ODNPs.

FIGS. 14A and 14B show HPLC separation of three 8-mers (FIG. 14A) and three 10-mers (FIG. 14B).

FIGS. 15A and 15B show the HPLC separation of one 4-mer, one 6-mer, three 8-mers and three 10-mers (FIG. 15A) and the elution of two 6-mers (FIG. 15B).

FIG. 16 is a control HPLC chromatogram.

FIG. 17 shows HPLC fractionation and detection of short fragments generated by restriction enzyme Fok I double digest.

FIG. 18 shows changes in fluorescence polarization for DNA samples genotyped with the FP-EcoN I assay.

FIG. 19 is a schematic diagram of the major components of immobilized ODNPs and a resulting amplicon of the present invention.

FIG. 20 is a schematic diagram of the major components of an interrupted restriction endonuclease recognition sequence. D₁D₂ . . . Dₘ is a specific nucleotide sequence consisting of m nuleotides, whereas D′₁D′₂ . . . D′ₘ is the complement sequence of D₁D₂ . . . Dₘ. The double-stranded fragment comprised of D₁D₂ . . . Dₘ and D′₁D′₂ . . . D′ₘ forms the first CRS (also referred to as “Region D”). N₁N₂ . . . Nₙ is a variable nucleotide sequence consisting of n nucleotides where any one of the nucleotide can contain any of the four bases (a, c, t, or g). N′₁N′₂ . . . N′ₙ is the complement of N₁N₂ . . . Nₙ and forms a VRS in combination of N₁N₂ . . . Nₙ (also referred to as “Region N”). Region E of immobilized ODNPs of the present invention constitutes a portion of Region N. S₁S₂ . . . Sᵢ is a specific nucleotide sequence consisting of i nucleotides, whereas S′₁S′₂ . . . S′ᵢ is the complement of S₁S₂ . . . Sᵢ. The double-stranded fragment comprised of S₁S₂ . . . Sᵢ and S′₁S′₂ . . . S′ᵢ forms the second CRS (also referred to as “Region S”).

FIG. 21 is a schematic diagram of various types of digestion products. Oligonucleotides (a), (b), (c) and (d) designate the following single-stranded oligonucleotides: (a) the immobilized strand that contains the first ODNP, (b) the complementary strand of oligonucleotide (a), (c) the non-immobilized strand that was a portion of an extension product of the first ODNP, and (d) the complementary strand of oligonucleotide (c).

### DETAILED DESCRIPTION OF THE INVENTION

The present invention provides methods, compositions, and kits for determining sequence information at a defined genetic loci in a target nucleic acid and for parallel measurement of genetic variations. As described in more detail below, the invention provides for the design, preparation and use of oligonucleotide primers (ODNPs) that can be extended in a manner that incorporates information about the nucleotide of interest into the extension product. The resulting product, e.g., amplicon, can then be analyzed by various methods, also described in more detail below, to determine the identity of the nucleotide of interest. This information is advantageously utilized in a variety of applications, as described herein, such as genetic analysis for hereditary diseases, tumor diagnosis, disease predisposition, forensics or paternity, crop cultivation and animal breeding, expression profiling of cell function and/or disease marker genes, and identification and/or characterization of infectious organisms that cause infectious diseases in plants or animals and/or that are related to food safety.

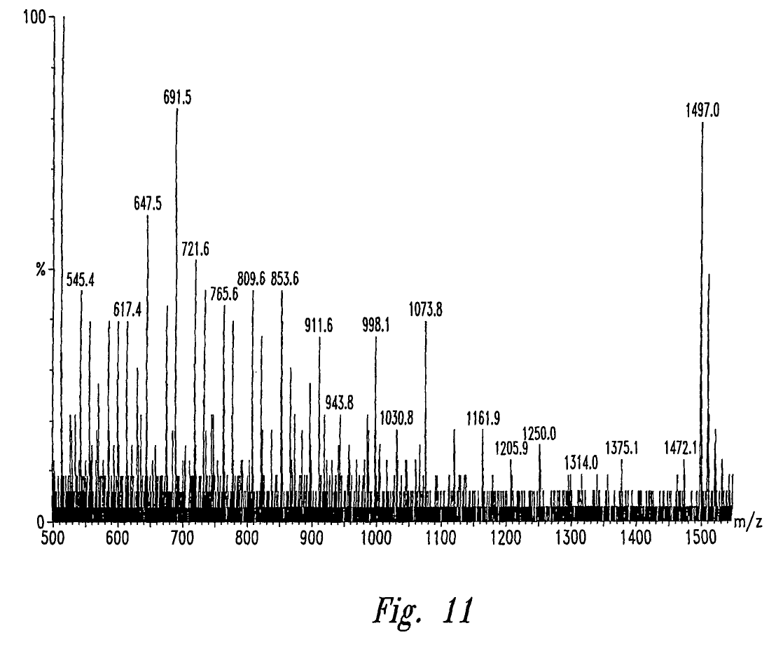

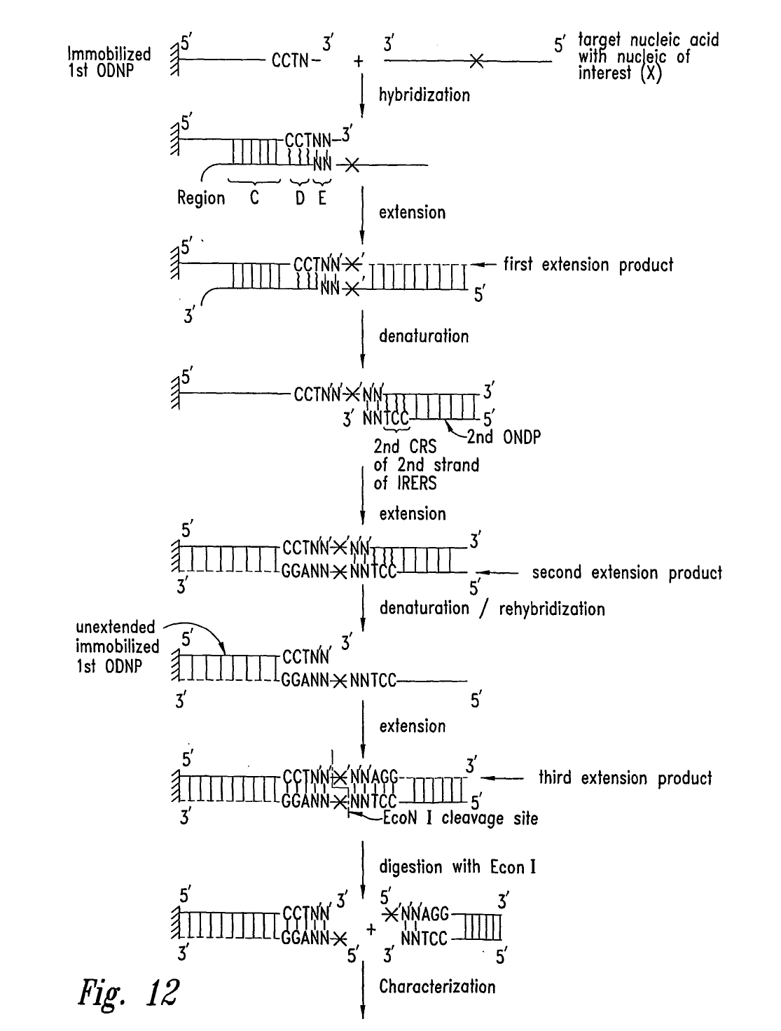

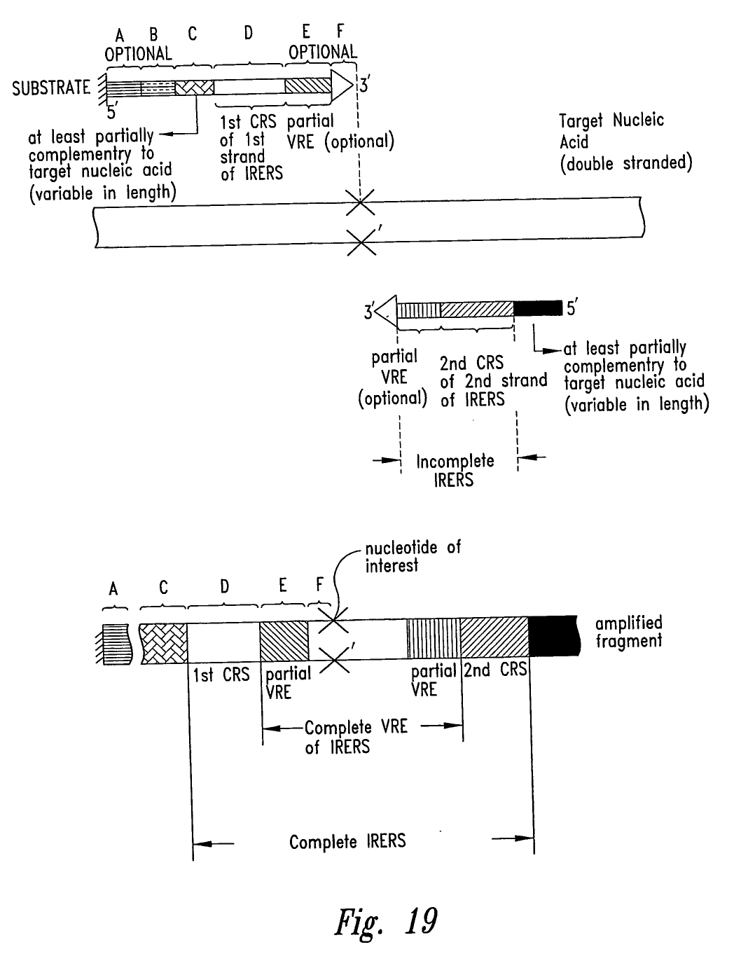

The ODNPs of the present invention each contain part of an interrupted restriction endonuclease recognition sequence (IRERS), defined in detail below. The interrupted segment of the restriction endonuclease recognition site (also referred to as “variable recognition sequence (VRS)”) may be one or more nucleotides in length and the sequence is variable (each position can contain any of the four bases (a, c, t, or g)). When extended and incorporated into an amplified fragment, the two primers together, in combination with the segment of target nucleic acid between them (i.e., VRS) form a single and complete IRERS. The primers are designed such that the nucleotide of interest in a target nucleic acid is located in the amplicon within the variable segment of the restriction endonuclease recognition site. The amplicon can then be digested to generate small fragments of nucleic acid that can be analyzed to determine the nucleotide of interest with great accuracy and sensitivity. The ODNPs of the present invention are shown schematically in FIG. 19. One primer of each ODNP pair is immobilized to a substrate to facilitate characterization of digested products. In FIG. 12, a diagram of the present invention is shown using the exemplary restriction endonuclease recognition sequence for EcoN I. One skilled in the art will appreciate that any interrupted restriction endonucleate recognition sequence may be used (see Table 2).

In various aspects, the present invention provides assays for determining the identity of a nucleotide at a predetermined location in a target nucleic acid molecule and for parallel measurement of genetic variations. In additional aspects, provided herein are compounds and compositions that are useful in performing such assays. In other aspects, the present invention provides compounds and compositions that, upon suitable characterization, identify the base at a predetermined location in a target nucleic acid or measure genetic variation in parallel. Still further aspects of the present invention are described hereinbelow.

### A. Conventions

Prior to providing a more detailed description of the present invention, it may be helpful to an understanding thereof to define a convention as used herein, as follows. The terms “3′” and “5′” are used herein to describe location of a particular site within a single strand of nucleic acid. When a location in nucleic acid is “3′ to” or “3′ of” a nucleotide of interest, this means that it is between the nucleotide of interest and the 3′ hydroxyl of that strand of nucleic acid. Likewise, when a location in a nucleic acid is “5′ to” or “5′ of” a nucleotide of interest, this means that it is between the nucleotide of interest and the 5′ phosphate of that strand of nucleic acid.

Also, as used herein, the word “a” refers to one or more of the indicated items unless the context clearly indicates otherwise. For instance, “a” polymerase refers to one or more polymerases.

### B. Methodology of the Present Invention

According to the present invention, the identity of a nucleotide of interest in a target nucleic acid molecule is determined by combining the, target with two primers. The first primer is immobilized to a substrate and hybridizes to and extends from a location 3′ of the nucleotide of interest in the target, so as to incorporate the complement of the nucleotide of interest in a first extension product. The second primer then hybridizes to and extends based on the first extension product, which is immobilized to the substrate via the first primer, at a location 3′ of the complement of the nucleotide of interest, so as to incorporate the nucleotide of interest in a second extension product. The second extension product then dissociates from the first extension product and thus from the substrate and re-hybridizes to another first primer molecule that has not extended. The non-extended first primer then extends from a location 3′ of the nucleotide of interest in the second extension product, so as to form, in combination with the second extension product, a double-stranded nucleic acid fragment. The first and second primers are designed to incorporate a portion of the recognition sequence of a restriction endonuclease (RE) that recognizes a partially variable interrupted nucleotide sequence, i.e., a sequence of the form D-N-S where D and S refer to specific nucleotide sequences essential for RE recognition (also referred to as the first constant recognition sequence (CRS) and the second CRS, respectively), and N is a sequence consisting of n viable nucleotides also required for RE recognition. The first primer incorporates the sequence D, the second primer incorporates the sequence S, and they are designed, in view of the target, to product a nucleic acid fragment where constant sequences D and S are separated by variable sequence N, where the nucleotide of interest is within region N. Action of the RE on the nucleic acid fragment provides a small nucleic acid fragment that is amendable to characterization, to thereby reveal the identity of the nucleotide of interest. The use of short nucleic acid (e.g., DNA) fragments is advantageous for numerous readout systems because amplicons produced during, e.g., a PCR amplification reaction, need not be tagged or labeled to facilitate detection.

The present invention also provides a method of parallel measurement of genetic variations. Genetic variations at defined positions in target nucleic acids are identified by combining the targets with two sets of ODNPs. The first set of ODNPs are immobilized to a substrate in distinct areas. Each ODNP of the two sets is designed to incorporate a portion of an IRERS (i.e., CRS) so that the extension and/or amplification product of an ODNP of the first set and its corresponding ODNP of the second set contains a complete IRERS. Because the ODNP of the first set and its corresponding ODNP of the second set are complementary to a portion of a target or the complement of the target at a location 3′ to a defined position where there is a possibility of a genetic variation, the extension and/or amplification product from the two ODNPs with the target as a template contains the nucleotide at the defined position. The extension and/or amplification product is then digested with a RE that recognizes the IRERS and the resulting small fragment is characterized. The genetic variation at the defined position is thereby identified. Because ODNPs are immobilized in distinct areas of the substrate, the above methods can be used to determine and/or measure genetic variations in multiple target nucleic acids in parallel.

The methods of the present invention have one or more of the following advantages: (1) very little target nucleic acid(s) is required; (2) multiple alleles may be assayed simultaneously in a “containerless” setting (i.e., different alleles need not be contained in distinct containers); (3) the ODNPs need not be labeled or tagged; (4) multiple measurements of genetic variations can be made in parallel; (5) the incorporation of nucleotides to be identified into small fragments is carried out with high fidelity; (6) no need exists for a dephosphorylation step after extension reactions as all the unincorporated oligonucleotides, dNTPs, etc. can be washed away; (7) signal is generated by the use of inexpensive labeled (e.g., fluorescent) dNTPs or rNTPs; and (8) no third primer is required as is the case for single nucleotide extension assays. These and other advantages will become more obvious with more detailed descriptions below.

### 1. Target Nucleic Acid Molecules

Methods, kits and compositions of the present invention typically involve or include a target nucleic acid molecule. The target nucleic acid of the present invention is any nucleic acid molecule about which nculeotide information is desired, and which can serve as a template for a primer extension reaction, i.e., can base pair with a primer.

The term “nucleic acid” refers generally to any molecule, preferably a polymeric molecule, incorporating units of ribonucleic acid or an analog thereof. The template nucleic acid can be either single-stranded or double-stranded. In one aspect, the template nucleic acid is DNA. In another aspect, the template is RNA. Suitable nucleic acid molecules are DNA, including genomic DNA, ribosomal DNA and cDNA. Other suitable nucleic acid molecules are RNA, including mRNA, rRNA and tRNA. The nucleic acid molecule may be naturally occurring, as in genomic DNA, or it may be synthetic, i.e., prepared based up human action, or may be a combination of the two.

A naturally occurring nucleic acid is obtained from a biological sample. The biological sample can be any sample that contains biological material (e.g., cells, tissues) from any organisms, including but not limited to, animals, higher plants, fungi, bacteria, and viruses. One type of preferred biological samples include one or more mammalian tissues, (for example blood, plasma/serum, hair, skin, lymph node, spleen, liver, etc) and/or cells or cell lines. The biological samples may comprise one or more human tissues and/or cells. Mammalian and/or human tissues and/or cells may further comprise one or more tumor tissues and/or cells. Another type of preferred biological samples contain viruses, such as pathogenic viruses (e.g., hepatitis A, B, or C), herpes virus (e.g., VZV, HSV-1, HAV-6, HSV-II, CMV, and Epstein Barr virus), adenovirus, influenza virus, echovirus, rhinovirus, comovirus, respiratory syncytical virus, mumps virus, measles virus, rubella virus, parvovirus, poliovirus, rabies virus, and flaviviruses. Other preferred biological samples contain genomic or episomic nucleic acids of pathogenic bacteria, particularly regions conferring drug resistance or useful for phylogenic characterization of the host (e.g., 16S rRNA). Such bacteria include, but is not limited to, chlamydia, rickettsial bacteria, mycobacteria, staphylococci, treptocci, pneumonococci, meningococci, conococci, klebsiella, proteus, serratia, pseudomonas, bacilli, cholera, tetanus, anthrax, plague, and Lymes disease bacteria.

The biological sample may contain biological material from a single organism, or a population of (i.e., two or more) organisms of a single species. Alternatively, the biological sample may contain biological material from organisms of two or more species.

Methodology for isolating populations of nucleic acids from biological samples are well known and readily available to those skilled in the art of the present invention. Exemplary techniques are described, for example, in the following laboratory research manuals: Sambrook et al., “Molecular Cloning” (Cold Spring Harbor Press, 3rd Edition, 2001) and Ausubel et al., “Short Protocols in Molecular Biology” (1999) (incorporated herein by reference in their entirety). Nucleic acid isolation kits are also commercially available from numerous companies which simplify and accelerate the isolation process.

A synthetic nucleic acid is produced by human intervention. At this time, many companies are in the business of making and selling synthetic nucleic acids that may be useful as the template nucleic acid molecule in the present invention. See, e.g., Applied Bio Products Bionexus (www.bionexus.net); Commonwealth Biotechnologies, Inc. (Richmond, Va.; www.cbi-biotech.com); Gemini Biotech (Alachua, Fla.; www.geminibio.com); INTERACTIVA Biotechnologie GmbH (Ulm, Germany; www.interactiva.de); Microsynth (Balgach, Switzerland; www.microsynth.ch); Midland Certified Reagent Company (Midland, Tex.; www.mcrc.com); Oligos Etc. (Wilsonville, Oreg.; www.oligosetc.com); Operon Technologies, Inc. (Alameda, Calif.; www.operon.com); Scandanavian Gene Synthesis AB (Köping, Sweden; www.sgs.dna); Sigma-Genosys (The Woodlands, Tex.; www.genosys.com); Synthetic Genetics (San Digeo, Calif.; www.syntheticgenetics.com, which was recently purchased by Epoch Biosciences, Inc. (Bothell, Wash.; www.epochbio.com); and many others.

The synthetic nucleic acid template may be prepared using an amplification reaction. The amplification reaction may be, for example, the polymerase chain reaction.

The synthetic nucleic acid template may be prepared using recombinant DNA means through production in one or more prokaryotic or eukaryotic organism such as, e.g., E. coli, yeast, Drosophila or mammalian tissue culture cell line.

The nucleic acid molecule may, and typically will, contain one or more of the ‘natural’ nucleotides, i.e., adenine (A), guanine (G), cytosine (C), thymine (T) and, in the case of an RNA, uracil (U). In addition, and particularly when the nucleic acid is a synthetic molecule, the target nucleic acid may include “unnatural” nucleotides. Unnatural nucleotides are chemical moieties that can be substituted for one or more natural nucleotides in a nucleotide chain without causing the nucleic acid to lose its ability to serve as a template for a primer extension reaction. The substitution may include either sugar and/or phosphate substitutions, in addition to base substitutions.

Such moieties are very well known in the art, and are known by a large number of names including, for example, abasic nucleotides, which do not contain a commonly recognized nucleotide base, such as adenine, guanine, cytosine, uracil or thymnine (see, e.g., Takeshita et al. “Oligonucleotides containing synthetic abasic sites,” J. Biol. Chem. 262:10171-10179 (1987); Iyer et al. “Abasic oligodeoxyribonucleoside phosphorothioates: synthesis and evaluation as anti-HIV-1 agents” Nucleic Acids Res. 18:2855-2859 (1990); and U.S. Pat. No. 6,117,657); base or nucleotide analogs (see, e.g., Ma et al., “Design and Synthesis of RNA Miniduplexes via a Synthetic Linker Approach. 2. Generation of Covalently Closed, Double-Stranded Cyclic HIV-1 TAR RNA Analogs with High Tat-Binding Affinity,” Nucleic Acids Res. 21:2585 (1993). Some bases are known as universal mismatch base analogs, such as the abasic 3-nitropyrrole); convertides (see, e.g., Hoops et al., Nucleic Acids Res. 25:4866-4871 (1997)); modified nucleotides (see, e.g., Millican et al., “Synthesis and biophysical studies of short oligodeoxynucleotides with novel modifications: A possible approach to the problem of mixed base oligodeoxynucleotide synthesis,” Nucleic Acids Res. 12:7435-7453 (1984); nucleotide mimetics; nucleic acid related compounds; spacers (see, e.g., Nielsen et al., Science 254:1497-1500 (1991); and specificity spacers (see, e.g., PCT International Publication No. WO 98/13527).

Additional examples of non-natural nucleotides are set forth in: Jaschke et al., Tetrahedron Tett. 34:301 (1993); Seela and Kaiser, Nucleic Acids Res. 15:3113 (1990) and Nucleic Acids Res. 18:6353 (1990); Usman et al., PCT International Patent Application No. PCT/US 93/00833; Eckstein, PCT International Patent Application No. PCT/EP91/01811; Sproat et al., U.S. Pat. No. 5,334,711, and Buhr and Matteucci, PCT International Publication No. WO 91/06556; Augustyns, K. A. et al., Nucleic Acids Res., 1991, 19, 2587-2593); and U.S. Pat. Nos. 5,959,099 and 5,840,876.

When the template nucleic acid molecule, and/or the primer used in the present method, contains a non-natural nucleotide, then a base-pair mismatch will occur between the template and the primer. The term “base-pair mismatch” refers to all single and multiple nucleotide substitutions that perturb the hydrogen bonding between conventional base pairs, e.g., G:C, A:T, or A:U, by substitution of a nucleotide with a moiety that does not hybridize according to the standard Watson-Crick model to a corresponding nucleotide on the opposite strand of the oligonucleotide duplex. Such base-pair mismatches include, e.g., G:C, G:T, G:A, G:U, C:C, C:A, C:T, C:U, T:T, T:U, U:U and A:A. Also included within the definition of base-pair mismatches are single or multiple nucleotide deletions or insertions that perturb the normal hydrogen bonding of a perfectly base-paired duplex. In addition, base-pair mismatches arise when one or both of the nucleotides in a base pair has undergone a covalent modification (e.g., methylation of a base) that disrupts the normal hydrogen bonding between the bases. Base-pair mismatches also include non-covalent modifications such as, for example, those resulting from incorporation of intercalating agents such as ethidium bromide and the like that perturb hydrogen bonding by altering the helicity and/or base stacking of an oligonucleotide duplex.

The template, in addition to containing nucleic acids or analogs thereof, also contains one or more natural bases of unknown identity. The present invention provides compositions and methods whereby the identity of the unknown nucleotide(s) becomes known. The base(s) of unknown identity is present at the “nucleotide loci” (or the “defined position”), refers to a specific nucleotide or region encompassing one or more nucleotides having a precise location on a target nucleic acid.

The base(s) to be identified in the target nucleic acid may be a mutation. The term “mutation” refers to an alteration in a wild-type nucleic acid sequence. Mutations may be in regions encoding proteins (exons) or may be in non-coding regions (introns or 5′ and 3′ flanking regions) of a target nucleic acid. Exemplary mutations in non-coding regions include regulatory mutations that alter the amount of gene product, localization of protein and/or timing of expression. The term “point mutations” refers to mutations in which a wild-type base (i.e., A, C, G or T) is replaced with one of the other bases at a defined nucleotide locus within a nucleic acid sample. They can be caused by a base substitution or a base deletion. A “frameshift mutation” is caused by a small deletion or insertion that, in turn, causes the reading frame to be shifted and, thus, a novel peptide to be formed. A “regulatory mutation” is a mutation in a region(s) of the gene not coding for protein, e.g., intron, 5′- or 3′-flanking, but affecting correct expression (e.g., amount of product, localization of protein, timing of expression). A “nonsense mutation” is a single nucleotide change resulting in a triplet codon (where mutation occurs) being read as a “STOP” codon causing premature termination of peptide elongation, i.e., a truncated peptide. A “missense mutation” is a mutation that results in one amino acid being exchanged for a different amino acid. Such a mutation may cause a change in the folding (3-dimensional structure) of the peptide and/or its proper association of other peptides in a multimeric protein.

The term “trinucleotide repeat” refers to a class of mutations that overlap with the chromosomal disorders, since large deletions in the “trinucleotide repeat” can be seen using cytological methods. A trinucleotide repeat is a 3-base-pair sequence of nucleic acid (typically DNA) in or around the gene which is reiterated tandemly (one directly adjacent to the next) multiple times. The mutation is observed when abnormal expansion of the repeat at variable levels results in the abnormal phenotype. The severity of the disorder can sometimes be correlated with the number of repeats in the expanded region, e.g., fragile X mental retardation syndrome, Huntington Disease, and myotonic dystrophy.

The nucleotide of interest, i.e., the nucleotide to be identified, may be a “single-nucleotide polymorphism” (SNP), which refers to any nucleotide sequence variation, preferably one that is common in a population of organisms and is inherited in a Medelian fashion. In a preferred embodiment, the SNP is either of two possible nucleotides, and there is no possibility of finding a third or fourth nucleotide identity at an SNP site.

Thus, a defined nucleotide locus within the target nucleic acid that comprises a nucleotide to be identified may contain a point mutation, single nucleotide polymorphism, deletion and/or insertion mutation. The target nucleic acid may also be a complement of such a mutated allele.

The term “polymorphism” or “genetic variation,” as used herein, refers to the occurrence of two or more genetically determined alternative sequences or alleles in a small region (ie., one to several (e.g., 2, 3, 4, 5, 6, 7, or 8) nucleotides in length) in a population. The allelic form occurring most frequently in a selected population is referred to as the wild type form. Other allelic forms are designated as variant forms. Diploid organisms may be homozygous or heterozygous for allelic forms.

The genetic variation may be associated with or cause diseases or disorders. The term “associated with,” as used herein, refers to the correlation between the occurrence of the genetic variation and the presence of a disease or a disorder. Such diseases include, but are not limited to, bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

Target nucleic acids may be amplified before being combined with ODNPs as described below. Any known methods for amplifying nucleic acids may be used. Exemplary methods, such as the use of Qbeta Replicase, Strand Displacement Amplification, transcription-mediated amplification, RACE, and one-sided PCR, are described in detail below.

### 2. Design of Oligonucleotide Primers (ODNPs)

Methods, kits and compositions of the present invention typically involve or include one or more ODNPs which generally contain one or more partial IRERSs and regions of complementarity with target nucleic acids. For the purpose of simplicity, the target nucleic acid is described as a single-stranded nucleic acid below. However, one of ordinary skill in the art would readily design the ODNP(s) of the present invention wherein the target nucleic acid is double-stranded. For instance, a double-stranded nucleic acid may be first denatured to form two single-stranded nucleic acids before being mixed with ODNPs.

The term “oligonucleotide” (ODN) refers to a nucleic acid fragment (typically DNA or RNA) obtained synthetically as by a conventional automated nucleic acid (e.g., DNA) synthesizer. Oligonucleotide is used synonymously with the term polynucleotide. The term “oligonucleotide primer” (ODNP) refers to any polymer having two or more nucleotides used in a hybridization, extension, and/or amplification reaction. The ODNP may be comprised of deoxyribonucleotides, ribonucleotides, or an analog of either. As used herein for hybridization, extension, and amplification reactions, ODNPs are generally between 8 and 200 bases in length. More preferred are ODNPs of between 12 and 50 bases in length and still more preferred are ODNPs of between 18 and 32 bases in length.

In one aspect, the present invention provides an immobilized ODNP useful for producing a portion of a target nucleic acid containing a nucleotide to be identified at a defined position in combination of a second ODNP. The immobilized ODNP (“the first ODNP” or “the forward primer”) comprises a nucleic acid sequence complementary to a nucleotide sequence of a target nucleic acid at a location 3′ to the defined position (“the first region of the target nucleic acid”), whereas the second primer (“the reverse primer”) comprises a nucleic acid sequence complementary to a nucleotide sequence of the complement of the target nucleic acid, also at a location 3′ to the defined position (“the first region of the complement”). The complementarity between the ODNPs and their corresponding target nucleic acid or the complement thereof need not be exact, but must be sufficient for the ODNPs to selectively hybridize with the target nucleic acid or the complement thereof such that the ODNPs are able to function as primers for extension and/or amplification using the target nucleic acid, or the complement thereof, as a template. Generally, each ODNP contains at least 5, preferably at least 7, more preferably at least 9, most preferably at least 11 nucleotides that are complementary to the target nucleic acid or the complement thereof. Because both ODNPs hybridize to a target nucleic acid or the complement thereof at a location 3′ to the defined position, the resulting extension and/or amplification products from the two ODNPs contains the nucleotide to be identified at the defined position.

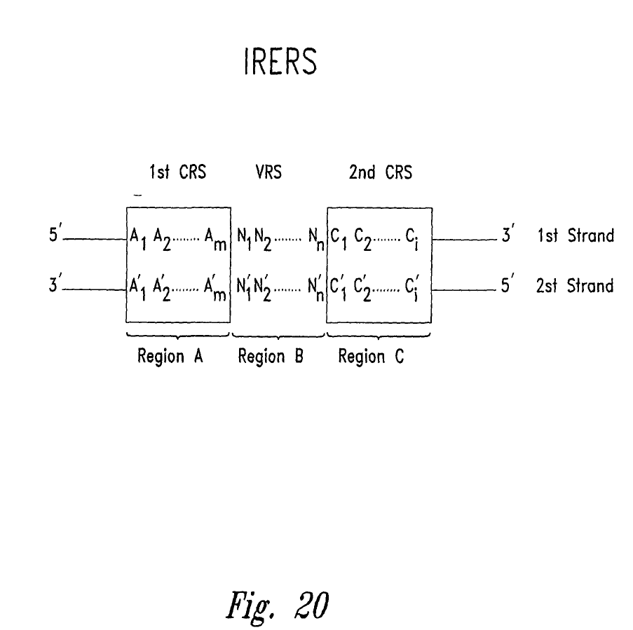

The first primer and the second primer each further comprises a partial IRERS, but not a complete IRERS, at a location 3′ to, or preferably at the 3′ terminus of, its nucleic acid sequence described above (i.e., the sequence complementary to the target nucleic acid or the complement thereof). As described in more detail below, a complete IRERS is a double-stranded oligonucleotide sequence comprising a first CRS and a second CRS linked with a VRS (FIG. 20). Generally, the first ODNP and the second ODNP comprise the first CRS of the first strand of the IRERS and the second CRS of the second strand of the IRERS, respectively. In addition, the first ODNP and the second ODNP are so spaced that (1) the extension and/or amplification product with the ODNP pair as primers and the target nucleic acid as a template contains a complete IRERS and (2) the nucleic acid to be identified is within the VRS. In other words, the number of nucleotides between the first and the second CRS is the exact number of nucleotides in the VRS so that the extension and/or amplification product from both ODNPs can be digested by a RE that recognizes the complete IRERS. The partial IRERS in each ODNP may or may not be complementary to the target nucleic acid.

In a preferred embodiment, the first primer and/or the second primer further contains one or more nucleotides that is complementary to the target nucleic acid or the complement thereof (“the second region of the target nucleic acid” and “the second region of the complement,” respectively) at a location 3′ to, or preferably the 3′ terminus of, the CRS. Such nucleotides are a portion of the VRS (FIG. 19). The number of the nucleotides between first and second regions of the target nucleic acid or the complement thereof may be larger or smaller, but preferably equal to, the number of nucleotides of ODNPs between their two regions that complementary to the target nucleic acids or the complement thereof.

Alternatively, the immobilized ODNP may comprise additional sequences (FIG. 19). In FIG. 19, regions A, B, C, D, E and F designate the following elements or sequences: region A—an optional linking element that links the 5′ terminus of the ODNP to a substrate; region B—an optional nucleotide sequence; region C—a nucleotide sequence complementary to a nucleotide sequence of a single-stranded target nucleic acid at a location 3′ to a defined position “X” of the target; region D—a first CRS of a first strand of a complete IRERS; region E—an optional nucleotide sequence; and region F—an optional gap of nucleotides. The complementarity between region C and the nucleotide sequence of the target need not be exact. Preferably, the nucleotide sequence of region E is complementary to a nucleotide sequence of the target near the nucleotide sequence of the target that is complementary to region C. The overall complementary between the ODNP and the target (via region C and region E, if applicable, of the ODNP) must be sufficient for the ODNP to selectively hybridize with the target so that the ODNP is able to function as a primer, for extension and/or amplification using the target as a template. The number of nucleotides within regions E and F is within the range 0 to n-1 so that the ODNP does not contain the nucleotide at the defined position. The number “n” designates the number of nucleotides within the VRS of the complete IRERS.

General techniques for designing sequence-specific primers are well known. For instance, such techniques are described in books, such as PCR Protocols: Current Methods and Application, edited by Bruce A. White, 1993; PCR Primer: A Laboratory Manual, edited by Carl W. Dieffenbach and Gabriela S. Dveksler, 1995; PCR (Basics: From Background to Bench, by McPherson et al.; PCR Applications: Protocols for Functional Geomics, edited by Michael A. Innis, 1999; PCR: Introduction to Biotechniques Series, by Newton and Graham, 1997; PCR Protocols: A Guide to Methods and Application, by Gelfand et al., 1990; PCR Strategies, by Michael A. Innis; PCR Technology: Current Innovations, by Griffin and Griffin, 1994; and PCR: Essential Techniques, edited by J. F. Burke. In addition, softwares for designing primers are also available, including Primer Master (see, Proutski and Holmes, “Primer Master: A new program for the design and analysis of PCR primers.” Comput. Appl. Biosci. 12:253-5 (1996)) and OLIGO Primer Analysis Software from Molecular Biology Insights, Inc. (Cascade, Colo., USA). The above references and description of softwares are incorporated herein by reference in their entirety.

### 3. Immobilization of ODNPs

Methods, kits and compositions of the present invention may involve or include an immobilized ODNP or an array of immobilized ODNPs. The ODNPs of the present invention can be non-covalently immobilized to a substrate. Alternatively and preferably, the ODNPs are covalently immobilized to a substrate.

The substrate to which the ODNPs of the present invention are immobilized is prepared from a suitable material. The substrate is preferably rigid and has a surface that is substantially flat. In some embodiments, the surface may have raised portions to delineate areas. The suitable material includes, but is not limited to, silicon, glass, paper, ceramic, metal, metalloid, plastics and plastic copolyers. Typical substrates are silicon wafers and borosilicate slides (e.g., microscope glass slides). An example of a particularly useful solid support is a silicon wafer that is usually used in the electronic industry in the construction of semiconductors. The wafers are highly polished and reflective on one side and can be easily coated with various linkers, such as poly(ethyleneimine) using silane chemistry. Wafers are commercially available from companies such as WaferNet, San Jose, Calif.

As noted above, the present invention provides arrays of ODNPs useful for producing portions of target nucleic acids containing nucleotides to be identified at defined positions and/or for parallel measurement of genetic variations. As used herein, an “array” refers to a collection of ODNPs that are placed on a solid support (also referred to “substrate”) in distinct areas. Each area is separated by some distance in which no nucleic acid or oligonucleotide is bound or deposited. In some embodiments, area sizes are 20 to 500 microns and the center to center distances of neighboring areas range from 50 to 1500 microns. The array of the present invention may contain 2-9, 10-100, 101-400, 401-1,000, or more than 1,000 distinct areas.

Depending on the contemplated application, one of ordinary skill in the art may vary the composition of immobilized molecules of the present array. For instance, the ODNPs described above (i.e., the immobilized ODNPs useful for producing a portion of a target nucleic acid containing a nucleotide to be identified) may or may not be immobilized to every distinct area of the array. Preferably, the ODNPs in a distinct area of an array are homogeneous. More preferably, the ODNPs in every distinct area of an array to which the ODNPs are immobilized are homogeneous. The term “homogeneous,” as used herein, indicates that each ODNP molecule in a distinct area has the same sequence as another ODNP molecule in the same area. Alternatively, the ODNPs in at least one of the distinct areas of an array are heterogeneous. The term “heterogeneous,” as used herein, indicates that at least one ODNP molecule in a distinct area has a different sequence from another ODNP molecule in the area. In some embodiments, molecules other than the ODNPs described above may also be present in some or all of distinct areas of an array. For instance, a molecule useful as an internal control for the quality of an array may be attached to some or all of distinct areas of an array. Another example for such a molecule may be a nucleic acid useful as an indicator of hybridization stringency. In other embodiments, the composition of ODNPs in every distinct area of an array is the same. Such an array may be useful in determining genetic variations in a particular gene in a selected population of organisms or in parallel diagnosis of a disease or a disorder associated with mutations in a particular gene.

Depending on the envisioned application, the immobilized ODNPs of the present invention may contain oligonucleotide sequences (i.e., region C and optional region E) complementary to various target nucleic acids. Such target nucleic acids include, but are not limited to, genes associated with hereditary diseases in animals, oncogenes, genes related to disease predisposition, genomic DNAs useful for forensics and/or paternity determination, genes associated with or rendering desirable features in plants or animals, and genomic or episomic DNA of infectious organisms. An array of the present invention may contain ODNPs complementary to a particular type of target nucleic acids in distinct areas. For example, an array may have an ODNP complementary to a first gene related to disease predisposition in a first distinct area, another ODNP complementary to a second gene also related to disease predisposition in a second distinct area, yet another ODNP complementary to a third gene also related to disease predisposition in a third distinct area, etc. Such an array is useful to determine disease predisposition of an individual animal (including a human) or a plant. Alternatively, an array may have ODNPs complementary to multiple types of target nucleic acids as to the functions of the targets.

In addition, an array may contain ODNPs as described above (i.e., the immobilized ODNPs useful for producing a portion of a target nucleic acid containing a nucleotide to be identified) each having the same region D (i.e., the region consisting of a CRS). Alternatively, an array may contain ODNPs having different CRSs, which are part of different IRERSs and thereby recognized by different REs.

In general, for successful performance in an array environment, the immobilized ODNPs must be stable and not dissociate during hybridization, washing or analysis. The density of the immobilized ODNPs must be sufficient for the subsequent analysis. For an array suitable for the present methods, typically 1000 to 10¹², preferably 1000 to 10⁶, 10⁶ to 10⁹, or 10⁹ to 10¹² ODNP molecules are immobilized in at least one distinct area. However, there must be minimal non-specific binding of target nucleic acids to the substrate. The immobilization process should not interfere with the ability of immobilized ODNPs to hybridize with target nucleic acids in a sample being analyzed. Thus, it is often best for only one point, ideally the 5′ termini of the ODNPs to be immobilized.

It is typically not desirable to have the ODNPs directly bound to the substrate. This is because an ODNP bound in this way is constrained in the way it may bind to target nucleic acids. To maximize the ability of a bound ODNP to hybridize with targets, a linking element (i.e., region A of the immobilized ODNP of FIG. 19) is typically positioned between the other regions of the ODNP and the substrate. The linking element comprises a chemical chain that serves to distance the other regions of the ODNP from the substrate. In certain embodiments, the linking elements are cleavable so that the portion of ODNP containing regions C and D detaches from the substrate upon the cleavage of region A. There are a number of ways to position a linking element. In one common approach, the substrate is coated with a polymeric layer that provides linking elements with a lot of reactive ends/sites. A common example is glass slides coated with polylysine, which are commercially available. Another example is substrates coated with poly(ethyleneimine) as described in Published PCT Application No. WO99/04896 and U.S. Pat. No. 6,150,103. In certain embodiments, regions C and D of the present ODNP are further distanced from the substrate via region B (FIG. 19). In other embodiments, however, the oligonucleotide portions of the ODNPs (i.e., optional region B, regions C and D, and optional region E) are directly immobilized to a substrate.

Generally, an ODNP may be immobilized to a substrate in the following two ways: (1) synthesizing the ODNP directly on the substrate (often termed “in situ synthesis”), or (2) synthesizing the ODNP separately and then position and bind it to the substrate (sometimes termed “post-synthetic attachment”). For in situ synthesis, the primary technology is photolithography. Briefly, the technology involves modifying the surface of a solid support with photolabile groups that protect, for example, oxygen atoms bound to the substrate through linking elements. This array of protected hydroxyl groups is illuminated through a photolithographic mask, producing reactive hydroxyl groups in the illuminated areas. A 3′-O-phosphoramidite-activated deoxynucleoside protected at the 5′-hydroxyl with the same photolabile group is then presented to the surface and coupling occurs through the hydroxyl group at illuminated areas. Following further chemical reactions, the substrate is rinsed and its surface is illuminated through a second mask to expose additional hydroxyl groups for coupling. A second 5′-protected, 3′-O-phosphoramidite-activated deoxynucleoside is present to the surface. The selective photo-de-protection and coupling cycles are repeated until the desired set of products is obtained. Detailed description of using photolithography in array fabrication may be found in the following patents or published patent applications: U.S. Pat. Nos. 5,143,854; 5,424,186; 5,856,101; 5,593,839; 5,908,926; 5,737,257; and Published PCT Patent Application Nos. WO99/40105; WO99/60156; WO00/35931.

The post-synthetic attachment approach requires a methodology for attaching pre-existing oligonucleotides to a substrate. One method uses the bio-streptavidin interaction. Briefly, it is well known that biotin and streptavidin form a non-covalent, but very strong, interaction that may be considered equivalent in strength to a covalent bond. Alternatively, one may covalently bind pre-synthesized oligonucleotide to a substrate. For example, carbodiimides are commonly used in three different approaches to couple DNA to solid supports. In one approach, the support is coated with hydrazide groups that are then treated with carbodiimide and carboxy-modified oligonucleotide. Alternatively, a substrate with multiple carboxylic acid groups may be treated with an amino-modified oligonucleotide and carbodiimide. Epoxide-based chemistries are also used with amine modified oligonucleotides. Detailed descriptions of methods for attaching pre-existing oligonucleotides to a substrate may be found in the following references: U.S. Pat. Nos. 6,030,782; 5,760,130; 5,919,626; published PCT Patent Application No. WO00/40593; Stimpson et al. Proc. Natl. Acad. Sci. 92:6379-6383 (1995); Beattie et al. Clin. Chem. 41:700-706 (1995); Lamture et al. Nucleic Acids Res. 22:2121-2125 (1994); Chrisey et al. Nucleic Acids Res. 24:3031-3039 (1996); and Holmstrom et al., Anal. Biochem. 209:278-283 (1993).

The primary post-synthetic attachment technologies include ink jetting and mechanical spotting. Ink jetting involves the dispensing of oligonucleotides using a dispenser derived from the ink-jet printing industry. The oligonucleotides are withdrawn from the source plate up into the print head and then moved to a location above the substrate. The oligonucleotides are then forced through a small orifice, causing the ejection of a droplet from the print head onto the surface of the substrate. Detailed description of using ink jetting in array fabrication may be found in the following patents: U.S. Pat. Nos: 5,700,637; 6,054,270; 5,658,802; 5,958,342; 6,136,962 and 6,001,309.

Mechanical spotting involves the use of rigid pins. The pins are dipped into an oligonucleotide solution, thereby transferring a small volume of the solution onto the tip of the pins. Touching the pin tips onto the substrate leaves spots, the diameters of which are determined by the surface energies of the pins, the oligonucleotide solution, and the substrate. Mechanical spotting may be used to spot multiple arrays with a single oligonucleotide loading. Detailed description of using mechanical spotting in array fabrication may be found in the following patents or published patent applications: U.S. Pat. Nos. 6,054,270; 6,040,193; 5,429,807; 5,807,522; 6,110,426; 6,063,339; 6,101,946; and published PCT Patent Application Nos. WO99/36760; 99/05308; 00/01859; 00/01798.

One of ordinary skill in the art would appreciate that besides the techniques described above, other methods may also be used in immobilizing ODNPs to a substrate. Descriptions of such methods can be found in, but are not limited to, the following patent or published patent applications: U.S. Pat. Nos. 5,677,195; 6,030,782; 5,760,130; 5,919,626; and published PCT Patent Application Nos. WO98/01221; WO99/41007; WO99/42813; WO99/43688; WO99/63385; WO00/40593; WO99/19341; WO00/07022. These patents and patent applications, as well as all the references (including patents, patent applications, and journal articles) cited above in this section (i.e., Immobolization of ODNPs) are incorporated herein by reference in their entirety.

### 4. Nucleic Acid Hybridization and Extension/Amplification

Methods, kits and compositions of the present invention may involve or include ODNPs (either immobilized or non-immobilized) that are hybridized to a target nucleic acid, where the ODNPs facilitate the production and/or amplification of a defined nucleotide locus within the target nucleic acid. The ODNPs and target nucleic acid are thus preferably combined under base-pairing condition. Selection of suitable nucleic acid hybridization and/or amplification conditions are available in the art by, e.g., reference to the following laboratory research manuals: Sambrook et al., “Molecular Cloning” (Cold Spring Harbor Press, 1989) and Ausubel et al., “Short Protocols in Molecular Biology” (1999) (incorporated herein by reference in their entirety).

Depending on the application envisioned, the artisan may vary conditions of hybridization to achieve desired degrees of selectivity of ODNP towards target sequence. For applications requiring high selectivity, relatively stringent conditions may be employed to form the hybrids, such as, e.g., low salt and/or high temperature conditions, such as from about 0.02 M to about 0.15 M salt at temperatures of from about 50° C. to about 70° C. Such selective conditions are relatively intolerant of large mismatches between the ODNP target nucleic acid.

Alternatively, hybridization of the ODNPs may be achieved under moderately stringent buffer conditions such as, for example, in 10 mM Tris, pH 8.3; 50 mM KCl; 1.5 mM MgCl₂ at 60° C. which conditions permit the hybridization of ODNP comprising nucleotide mismatches with the target nucleic acid. The design of alternative hybridization conditions is well within the expertise of the skilled artisan.

After being hybridized to the target, the ODNPs are extended with the target or the complement thereof as a template using various methodologies known in the art, such as the polymerase chain reaction (PCR) and modified ligase chain reaction (LCR). For the purpose of simplicity, the target nucleic acid is described as a single-stranded nucleic acid below. However, one of ordinary skill in the art would readily extend the ODNPs of the present invention wherein the target nucleic acids are double-stranded. For instance, a double-stranded target nucleic acid may first be denatured to two single-stranded nucleic acids before being mixed with ODNPs.

The present invention provides a method to obtain a portion of a target nucleic acid that contains a defined nucleotide locus and a complete IRERS and is immobilized to a substrate. To do so, at least three runs of extension reactions from an immobilized ODNP (“the first primer”) and a corresponding non-immobilized ODNP (“the second primer”) described above need be carried out (FIG. 12). In addition, there are fewer target nucleic acid molecules than the first primer molecules in the reaction mixture so that not all of the first primer molecules hybridize to target molecules during the first run of extension. Briefly, the first run of extension is for the first primer having a first CRS to incorporate the complement of the nucleotide of interest in the first extension product. The second primer having a second CRS then hybridizes to and extends using the first extension product as a template and thereby incorporates the nucleotide of interest and the first CRS in a second extension product. The second extension product then dissociates from the first extension product and re-hybridizes to another first primer molecule that has not extended. The non-extended first primer then extends from a location 3′ to the nucleotide of interest in the second extension product, so as to form, in combination with the second extension product, a double-stranded nucleic acid fragment. Because the first ODNP and its corresponding second ODNP are spaced in a distance of the same number of base pairs as that of the VRS, the double-stranded nucleic acid fragment resulting from the three runs of extensions contains a complete IRERS.

While three runs of extension reactions are sufficient to produce a fragment containing a defined nucleotide locus within a target nucleic acid and a complete IRERS, preferably, more than three extension reactions are conducted to amplify the fragment. As one of ordinary skill in the art would appreciate, in the subsequent runs of extensions, the first primer can hybridize to and extend using any of the target nucleic acid, the second extension product, and the complement of the third extension product as a template, as a template. Similarly, in the subsequent runs of extensions, the second primer can hybridize to and extend using either the first extension product or the third extension product as a template. However, because the third extension product and the complement thereof are shorter than any of the target nucleic acid, the first extension product and the second extension product, they are the preferred templates for subsequent extension reactions from either the first or the second ODNPs. This is because the extension efficiency with a short fragment as a template is higher than that with a large fragment as a template. With the increase of the number of extension reactions, the double stranded fragment containing both the nucleotide to be identified and a complete IRERS accumulates more quickly than other molecules in the reaction mixture. Such accumulation increases the sensitivity of subsequent characterization of the fragment after being digested with a RE that recognizes the complete IRERS.

The present invention also provides a method to obtain portions of target nucleic acids that contain defined nucleotide loci and complete IRERSs and are immobilized to a substrate. To do so, target nucleic acids are mixed with two sets of ODNPs: a first set that is immobilized to a substrate and thereby form an ODNP array and a second set that is not immobilized to any solid support. Each ODNP of the two sets is designed to incorporate a portion of an IRERS (i.e., CRS) as described in detail above so that the extension and/or amplification product of an ODNP of the first set and its corresponding ODNP of the second set contains a complete IRERS. Because the ODNP of the first set and its corresponding ODNP of the second set are complementary to a portion of a target or the complement of the target at a location 3′ of a defined position, the extension and/or amplification product from the two ODNPs with the target as a template contains the nucleotide at the defined position.

The extension/amplification reaction can be carried out using any extension/amplification method known in the art, including PCR methods. For instance, U.S. Pat. Nos. 4,683,195, 4,683,202 and 4,800,159 all describe PCR methods. In addition, PCR methods are also described in several books, e.g., PCR Protocols: Current Methods and Application, edited by Bruce A. White, 1993; PCR Primer: A Laboratory Manual, edited by Carl W. Dieffenbach and Gabriela S. Dveksler, 1995; PCR (Basics: From Background to Bench, by McPherson et al.; PCR Applications: Protocols for Functional Geomics, edited by Michael A. Innis, 1999; PCR: Introduction to Biotechniques Series, by Newton and Graham, 1997; PCR Protocols: A Guide to Methods and Application, by Gelfand et al., 1990; PCR Strategies, by Michael A. Innis; PCR Technology: Current Innovations, by Griffin and Griffin, 1994; and PCR: Essential Techniques, edited by J. F. Burke. Briefly, an excess of deoxynucleoside triphosphates is added to a reaction mixture along with a DNA polymerase (e.g., Taq or Pfu polymerase). If the sample contains a target nucleic acid that hybridizes with ODNPs, the polymerase will cause the ODNPs to be extended along the target nucleic acid sequence by adding on nucleotides. By raising and lowering the temperature of the reaction mixture, the extended ODNPs will dissociate from the target to form reaction products, excess ODNPs will bind to the target and to the reaction product and the process is repeated.

Exemplary PCR conditions according to the present invention may include, but are not limited to, the following: 100 μl PCR reactions comprise 100 ng target nucleic acid; 0.5 μM of each first ODNP and second ODNP; 10 mM Tris, pH 8.3; 50 mM KCl; 1.5 mM MgCl₂; 200 μM each dNTP; 4 units Taq™ DNA Polymerase (Boehringer Mannheim; Indianapolis, Ind.), and 880 ng TaqStart™ Antibody (Clontech, Palo Alto, Calif.). Exemplary thermocycling conditions may be as follows: 94° C. for 5 minutes initial denaturation; 45 cycles of 94° C. for 30 seconds, 60° C. for 30 seconds, 72° C. for 1 minute; final extension at 72° C. for 5 minutes. Exemplary nucleic acid polymerases may include one of the thermostable DNA polymerases that are readily available in the art such as, e.g., Taq™, Vent™ or PFU™. Depending on the particular application contemplated, it may be preferred to employ one of the nucleic acid polymerases having a defective 3′ to 5′ exonuclease activity.

An alternative way to make and/or amplify a fragment that contains a nucleotide to be identified at a defined position and a complete IRERS and is immobilized to a substrate is by a modified ligase chain reaction, referred to herein as the gap-LCR (Abravaya, et al., Nucleic Acids Res. 23:675-682 (1995)). This method requires four ODNPs (“primer A,” “primer B,” “primer C,” and “primer D”), wherein primer A is immobilized to a substrate. Briefly, in the presence of a target nucleic acid, primers A and B will bind to the target located 5′ and 3′ of (on either side of) the defined position in the target, respectively; whereas primers C and D will bind to the complement of the target located 5′ and 3′ of the complement of the nucleotide of interest, respectively. In the presence of a polymerase and a ligase, the gaps between primers A and B and between primers C and D will be filled in and each pair of primers (i.e., primers A and B, or primers C and D) ligated to form a single unit. By temperature cycling, as in PCR, bound ligated units dissociate from the target and then serve as “target sequences” for ligation of excess ODNP pairs. In addition, each of the four ODNPs contains a CRS and is spaced from each other to ensure the ligated units contain a complete IRERS.

Gap-LCR may be used to produce immobilized fragments that contain nucleotides to be identified within target nucleic acids and complete IRERSs in parallel. Briefly, an array of primer As is used in combination of sets of primer Bs, Cs and Ds. Each primer A on the array will make and/or amplify a portion of a target nucleic acid that further contains a complete IRERS in the presence of its corresponding primers B, C and D, a polymerase, and a ligase. Accordingly, multiple immobilized fragments will be obtained that contain portions of target nucleic acids with different sequences.

As indicated above, gap-LCR uses both a nucleic acid polymerase enzyme and a nucleic acid ligase enzyme to drive the reaction. Exemplary nucleic acid polymerases may include one of the thermostable DNA polymerases that are readily available in the art such as, e.g., Taq™, Vent™ or PFUT™. Exemplary nucleic acid ligases may include T4 DNA ligase, or the thermostable Tsc or Pfu DNA ligases. U.S. Pat. No. 4,883,750, incorporated herein by reference in its entirety, describes an alternative method of amplification similar to LCR for binding ODNP pairs to a target sequence.

Exemplary gap-LCR conditions may include, but are not limited to, the following: 50 μl LCR reactions comprise 500 ng DNA; a buffer containing 50 mM EPPS, pH 7.8, 30 mM MgCl₂, 20 mM K⁺, 10 μM NAD, 1-10 μM gap filling nucleotides, 30 nM each oligonucleotide primer, 1 U Thermus flavus DNA polymerase, lacking 3′→5′ exonuclease activity (MBR, Milwukee, Wis.), and 5000 U T. thermophilus DNA ligase (Abbott Laboratories). Cycling conditions may consist of a 30 s incubation at 85° C. and a 30 s incubation at 60° C. for 25 cycles and may be carried out in a standard PCR machine such as a Perkin Elmer 9600 thermocycler.

In addition to the PCR and gap-LCR techniques described above, a number of other template dependent methodologies may be used either to amply target nucleic acids before combining the target nucleic acids with the ODNPs of the present invention. Alternatively, such methodologies may be used, in combination of the immobilized ODNPs or the array of ODNPs described above, to produce a fragment containing a portion of a target nucleic acid with a defined nucleotide locus and a complete IRERS. For instance, Qbeta Replicase, described in PCT Intl. Pat. Appl. Publ. No. PCT/US87/00880, incorporated herein by reference in its entirety, may alternatively be used with, methods of the present invention. By this method, a replicative sequence of RNA that has a region complementary to that of a target is added to a sample in the presence of an RNA polymerase. The polymerase will copy the replicative sequence that can then be detected.

Alternatively, Strand Displacement Amplification (SDA) may be employed to achieve isothermal amplification of nucleic acids. By this methodology, multiple rounds of strand displacement and synthesis, i.e. nick translation, are utilized. A similar method, called Repair Chain Reaction (RCR) is another method of amplification which may be useful in the present invention and involves annealing several ODNPs throughout a region targeted for amplification, followed by a repair reaction in which only two of the four bases are present. The other two bases can be added as biotinylated derivatives for easy detection. A similar approach is used in SDA.

Other nucleic acid amplification procedures include transcription-based amplification systems (TAS) (also referred to as transcription-mediated amplification, or TMA) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing an ODNP that has sequences specific to the target sequence. Following polymerization, DNA/RNA hybrids are digested with RNase H while double stranded DNA molecules are heat-denatured again. In either case the single stranded DNA is made filly double stranded by addition of a second target-specific ODNP, followed by polymerization. The double stranded DNA molecules are then multiply transcribed by a polymerase such as one of the RNA polymerases that are readily available in the art, e.g., SP6, T3, or T7. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, discloses a nucleic acid amplification process involving cyclically synthesizing single-stranded RNA (“ssRNA”), ssDNA, and double-stranded DNA (dsDNA), which may be used in accordance with the present invention. The ssRNA is a first template for a first ODNP, which is elongated by reverse transcriptase (RNA-dependent DNA polymerase). The RNA is then removed from resulting DNA:RNA duplex by the action of ribonuclease H (RNase H, an RNase specific for RNA in a duplex with either DNA or RNA). The resultant ssDNA is a second template for a second ODNP, which also includes the sequences of an RNA polymerase promoter (exemplified by T7 RNA polymerase) 5′ to its homology to its template. This ODNP is then extended by DNA polymerase (exemplified by the large “Klenow” fragment of E. coli DNA polymerase I), resulting as a double-stranded DNA (“dsDNA”) molecule, having a sequence identical to that of the original RNA between the ODNPs and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, disclose a nucleic acid sequence amplification scheme based on the hybridization of a promoter/ODNP sequence to a target single-stranded DNA (“ssDNA”) followed by transcription of many RNA copies of the sequence. This scheme is not cyclic; since new templates are not produced from the resultant RNA transcripts. Other amplification methods include “RACE” (Frohman, 1990), “rolling cycle,” (Thomas et al., Arch. Pathol. Lab Med. 123:1170-1176 (1999), Hatch et al. Genet. Anal. 15:35-40 (1999), Baner et al. Nucleic Acids Res. 26:5073-5078; Nakai, J. Biol. Chem 268:23997-24004 (1993)) and “one-sided PCR” (Ohara, 1989) which are well-known to those of skill in the art.

In most circumstances, hybridization and amplification conditions for reactions involving immobilized ODNPs (e.g., the ODNPs on an oligonucleotide array) are similar to those involving ODNPs in solution. One of ordinary skill in the art would be able to optimize those conditions in view of a particular ODNP and its corresponding target nucleic acid. Exemplary hybridization and/or amplification conditions may be found in U.S. Pat. Nos. 6,013,449, 6,156,502, 5,858,659, 5,424,186, 5,902,723, 6,045,996, 6,087,112, 6,156,501 and published PCT Patent Applications Nos. WO92/10092, 95/11995. All of these patents or patent applications are incorporated herein by reference.

### 5. Restriction Endonucleases and Digestion Conditions

Methods, kits and compositions of the present invention typically involve or include one or more interrupted restriction endonucleases. The term “restriction endonuclease” (RE), refers to the class of nucleases that bind to unique double-stranded nucleic acid sequences and that generate a cleavage in the double-stranded nucleic acid that results in either blunt, double stranded ends, or single stranded ends with either a 5′ or a 3′ overhang. As used herein, the term ”interrupted restriction endonuclease recognition sequence” (IRERS) is defined as a restriction endonuclease recognition site that is comprised of a “first constant recognition sequence (CRS),” a “second CRS,” and a “variable recognition sequence (VRE)” that links the first and second CRSs (FIG. 20). According to the present invention, “first CRS” (also referred to as “Region D”) is defined as that region of the IRERS that contains the constant (not variable) nucleotides of the IRERS that are located 5′ of the VRE of the IRERS. “Second CRS” (also referred to as “Region S”) is defined as that region of the IRERS that contains the constant (not variable) nucleotides of the IRERS that are located 3′ of the VRE of the IRERS. According to the present invention, the “VRE” (also referred as “Region N”) is defined as the stretch of one or more variable nucleotides that are located between the first and second CRSs.

The term “Bsl I” refers to an exemplary RE that binds to a unique nucleic acid sequence that is composed of 5′-CCNNNNNNNGG-3′ where N is an undefined nucleotide base or analog thereof, and that cleaves double-stranded nucleic acid. The cleavage site is as follows:

5′-CCNNNNN/NNGG-3′ (SEQ ID NO. 1)

3′-GGNN/NNNNNCC-5′ (SEQ ID NO. 2)

where the bottom and top strands are cleaved 4 bases in from the 3′-OH ends (“/” indicates the cleavage sites). In one aspect of the present invention, the base to be identified, e.g., the mutation or SNP, is positioned within the middle three “Ns” comprising the 3′overhang. In another aspect, the base to be identified is positioned within the 6^{th} nucleotide from the 5′ end of the top strand. Alternatively, the base to be identified may be at any other positions within the variable recognition sequence.

The term “EcoN I” refers to another exemplary RE that binds to a unique nucleic acid sequence composed of 5′-CCTNNNNNAGG-3′ (SEQ ID NO. 3) where N is an undefined nucleotide base or analog thereof, and that cleaves double-stranded nucleic acid. The cleavage site is as follows:

5′-CCTNN/NNNAGG-3′ (SEQ ID NO. 3)

3′-GGANNN/NNTCC-5′ (SEQ ID NO. 4)

where the bottom and top strands are cleaved 6 bases in from the 3′-OH ends (“/” indicates the cleavage sites). In one aspect of the present invention, the base to be identified is positioned at the 6^{th} nucleotide from the 5′ end of the top strand.

Any restriction endonuclease that recognizes an interrupted restriction endonuclease recognition sequence can be used in the present invention. Some of such enzymes are commercially available from numerous companies such as, e.g., New England Biolabs Inc. (Beverly, Mass.; www.neb.com); Stratagene (La Jolla, Calif.; www.stratagene.com), Promega (Madison, Wis.: www.promega.com), and Clontech (Palo Alto, Calif.; www.clontech.com). Non-commercially available restriction enzymes may be isolated and/or purified based on the teaching available in the art. For instances, the following articles describe the isolation and/or purification of several non-commercially available restriction enzymes suitable for the present invention and are incorporated herein in their entirety by reference: for restriction enzyme ApaB I, Grones and Turna, Biochim. Biophys. Acta 1162:323-325 (1993), Grones and Turna, Biologia (Bratisl) 46:1103-1108 (1991); for EcoH I, Glatman et al., Mol. Gen. Mikrobiol. Virusol. 3:32 (1990); for Fmu I, Rebentish et al. Biotekhnologiya 3:15-16 (1994); for HpyB II, FEMS Microbial. Lett. 179:175-180 (1999); for Sse8647 I, Nomura et al., European Patent Application No. 0698663 A1, Ishino et al., Nucleic Acids Res. 23: 742-744 (1995); for Unb I, Kawalec et al., Acta Biochim 44:849-852 (1997); for VpaK11A I, Miyahara et al. J. Food. Hyg. Sci. Japan 35:605-609 (1994).

Exemplary REs suitable for use in the present invention and their corresponding recognition sequences are presented in Table 1. It will be apparent to one of ordinary skill in the art, however, that REs available in the art that recognizes IRERSs, but are not included in Table 1, may be equally suitable depending on the particular application contemplated.

TABLE 1
Exemplary IRERSs and Their Corresponding REs

| | |
|---|---|
| RE | RECOGNITION SEQUENCE |
| Ahd I | GACNNN/NNGTC (SEQ ID NO. 5) |
| AlwN I | CAGNNN/CTG |
| Ava II | G/GWCC |
| Bgl I | GCCNNNN/NGGC (SEQ ID NO. 6) |
| Glp I | GC/TNAGC |
| Cac8 I | GCN/NGC |
| Dde I | C/TNAG |
| Dra III | CACNNN/GTG |
| EcoN I | CCTNN/NNNAGG (SEQ ID NO. 3) |
| Hinf I | G/ANTC |
| Hpy166 II | GTNNAC |
| Nci I | CC/SGG |
| PpuM I | RG/GWCCY |
| Sau96 I | G/GNCC |
| Sty I | C/CWWGG |
| Tfi I | G/AWTC |
| Tth111 I | GACN/NNGTC |
| Xmn I | GAANN/NNTTC (SEQ ID NO. 7) |

A nucleic acid fragment containing a portion of target nucleic acid with a defined nucleotide locus and a complete IRERS is digested (or cleaved) by a RE that recognizes the IRERS. Conditions for storage and use of restriction endonucleases used according to the present invention are readily available in the art, for example, by reference to one of the laboratory manuals such as Sambrook et al., supra and Ausubel et al., supra.

Briefly, the number of units of IPRE added to a reaction may be calculated and adjusted according to the varying cleavage rates of nucleic acid substrates. 1 unit of restriction endonuclease will digest 1 ug of substrate nucleic acid in a 50 μl reaction in 60 minutes. Generally, fragments (e.g., amplicons) may require more than 1 unit/ug to be cleaved completely. The restriction enzyme buffer is typically used at 1× concentration in the reaction. Some restriction endonucleases require bovine serum albumin (BSA) (usually used at a final concentration of 100 μg/ml for optimal activity). Restriction endonucleases that do not require BSA for optimal activity are not adversely affected if BSA is present in the reaction.

For an array of ODNPs having different CRSs, the immobilized extension/amplification products described above may be digested by different REs that recognize various IRERSs of which the CRSs of the ODNPs are components, if the digestion conditions for the REs are similar. However, if the different REs can not function well together, they may be applied to the array sequentially. Between each run of digestion, the buffer for a previous RE will be washed off the array, and the array will then be put into another buffer suitable for a next RE.

Most restriction enzymes are stable when stored at −20° C. in the recommended storage buffer. Exposure to temperatures above −20° C. should be minimized whenever possible. All restriction endonucleases should be kept on ice when not otherwise being stored in the freezer. Enzymes should always be the last component added to a reaction.

The recommended incubation temperature for most restriction endonucleases is about 37° C. Restriction endonucleases isolated from thermophilic bacteria require higher incubation temperatures, typically ranging from 50° C. to 65° C. Incubation time may often be shortened if an excess of restriction endonuclease is added to the reaction. Longer incubation times are often used to allow a reaction to proceed to completion with fewer units of restriction endonuclease.

### 6. Methodologies for Characterizing Short Nucleic Acid Fragments

The present invention provides methodology whereby a fragment is cleaved using a restriction endonuclease, so as to generate a short (also referred to as “small”) nucleic acid fragment. This short nucleic acid fragment contains information which, upon characterization of the fragment, allows one to determine the identity of the nucleotide(s) of interest in the target nucleic acid. Thus, the present invention transfers information about the nucleotide(s) of interest from a relatively large target nucleic acid into a relatively small nucleic acid fragment. In this way, the nucleotide(s) of interest is made to constitute a relatively large portion of the bases in a nucleic acid, such that characterization of the nucleic acid (fragment) is more readily able to reveal information about the nucleotide(s) of interest. In particular, a direct and complete characterization of the small nucleic acid fragment can be obtained (which is often practically impossible for a large target nucleic acid) which will reveal the identity of the nucleotide(s) of interest.

Thus, as discussed in detail hereinabove, methods according to the present invention employ, inter alia, the steps of using appropriate primer(s) and a target nucleic acid to prepare an intermediate structure (e.g., an amplicon) that is digested with a suitable restriction endonuclease to produce one or more small nucleic acid fragments. One or more of these fragments is then characterized to obtain partial or complete base sequence information about the fragment, including identification of the nucleotide(s) of interest in the target nucleic acid.

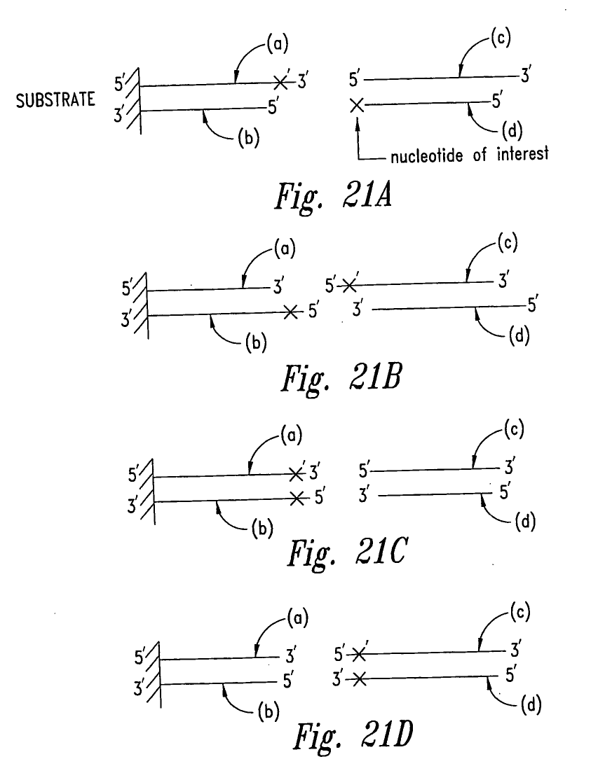

Any digestion product that contains the nucleotide(s) at a defined location or the complement thereof may be characterized to determine the identity of the nucleotide(s). As shown in FIG. 21, the nucleotide and/or its complement may reside in a digestion product immobilized to a substrate. Alternatively, the nucleotide and/or its complement may be in a digestion product in solution. There are three types of immobilized digestion products (double-stranded) that contain the nucleotide of interest and/or its complement: (1) a product having the complement of the nucleotide of interest within a 3′ overhang (FIG. 21, panel A); (2) a product having the nucleotide of interest within a 5′ overhang (FIG. 21, panel B); and (3) a product having both the nucleotide of interest and its complement (FIG. 21, panel C). Likewise, there are three types of digestion products in solution that contain the nucleotide of interest and/or its complement: (1) a product having the nucleotide of interest in a 3′ overhang (FIG. 21, panel A); (2) a product having the complement of the nucleotide of interest in a 5′ overhang (FIG. 21, panel B); and (3) a product having both the nucleotide of interest and its complement (FIG. 21, panel D). The first and second products (either immobilized or in solution) are resulted from digestions of REs that produce a 3′ overhang and a 5′ overhang, respectively. However, the third product (either immobilized or in solution) can be resulting from a digestion of a RE that produces any of the following ends: a 3′ overhang, a 5′ overhang and a blunt end.

The present invention allows the characterization of digestion products either on a solid substrate (i.e., for the immobilized digestion products) or in solution (i.e., for the non-immobilized digestion products). The immobilization of half of the digestion products eliminates, or reduces the extent of, fractionation needed before the characterization. For instance, the characterization of oligonucleotide (a) (i.e., the immobilized strand that contains the first ODNP) does not require any fractionation steps prior to its characterization as both the non-immobilized double-stranded digestion product and oligonucleotide (b) (i.e, the complementary strand of oligonucleotide (a)) may be washed off the substrate to which oligonucleotide (a) is bound after denaturation (FIG. 21). Similarly, the characterization of oligonucleotide (b) does not require any fractionation steps as oligonucleotide (b) can be separated from the non-immobilized double-stranded digestion product by washing off the substrate to which oligonuleotide (b) is bound via its base-pairing with oligonucleotide (a) and further separated from oligonucleotide (a) by denaturation. For non-immobilized digestion products, oligonucleotide (c) (i.e., the non-immobilized strand that was a portion of an extension product of the first ODNP) and its complementary strand oligonucleotide (d) may not need to be separated from each other before their characterization in certain embodiments. Even if separation is necessary in some other embodiments, the immobilization of oligonucleotides (a) and (b) simplifies the separation process as two, instead of four, single-stranded oligonucleotides need to be fractionated.

If various digestion products are not immobilized prior to their characterization and an array is used to generate these digestion products, the array may be designed to physically separate areas that contain different digestion products from each other. Any partitioning means suitable for this purpose may be used. For instance, the array may have raised portions to delineate distinct areas. Alternatively, the array may have lowered portions (e.g., wells as in microtiter plates) to define distinct areas. The digestion products that are not immobilized prior to their characterization include oligonucleotides (c) and (d), oligonucleotide (b) after its dissociation from oligonucleotide (a), and oligonucleotide (a) containing a cleavable linking element after its cleavage.

The characterization of a nucleic acid fragment (i.e., a digest product) can be done directly, that is, without the need to incorporate a tag or label into the fragment. Alternatively, in some embodiments, it may be advantageous to add one or more detectable labels.

### a. Direct Characterization

The present invention transfers information about nucleotide(s) of interest from a relatively large target nucleic acid into a relatively small nucleic acid fragment. Such information transfer allows direct characterization of the small fragment in many instances. For example, small nucleic acid fragments are amenable to direct detection by a variety of mass spectrometric methodologies (as discussed herein below) as well as by ultraviolet (UV) absorption.

In many instances according to the present invention, the complete nucleotide sequence, with the exception of a single nucleotide, will be known for the short nucleic acid fragment even before it is formed. The issue then becomes detecting the nucleotide of interest over the “noise” created while concurrently detecting the other bases. However, if the identity of the other nucleotides is known and their signal in the detection method is known, then this signal can be subtracted from the overall signal for the fragment, to leave information about the nucleotide of interest. This approach is essentially adopted in using mass spectrometry to characterize the small nucleic acid fragment. Other suitable methods, as discussed in detail herein, include determining the mass-to-charge ratio of the small nucleic acid fragment(s), by measuring fluorescence polarization and/or by quantifying ultraviolet (UV) absorption.

In some instances, characterizing a small nucleic acid fragment may entail simply determining the sizes of these single-strand fragments, and from this information the skilled artisan can deduce whether a target nucleic acid contains one or more mutations at a defined nucleotide locus. It will be apparent that the size of a single-strand fragment may be determined by numerous methods that are readily available in the art. Exemplary methods disclosed herein, including methods for measuring the size and/or molecular weight of a single-strand nucleic acid fragment, include, but are not limited to fluorescence including fluorescence polarization (FP), mass spectrometry (MS), ultraviolet (UV) absorption, cleavable mass tags, TaqMan (homogeneous), fluorescence resonance energy transfer (FRET), colormetric, luminescence and/or fluorescence methodologies employing substrates for horseradish peroxidase (HRP) and/or alkaline phosphatase (AP), as well as methods employing radioactivity.

In certain embodiments of the present invention, Mass Spectrometry (MS) may be employed for characterizing a strand of a small (short) nucleic acid fragments comprising the nucleotide locus of the target nucleic acid. MS may be particularly advantageous in those applications in which it is desirable to eliminate a fractionation step prior to detection. Alternatively, MS may also be employed in conjunction with a fractionation methodology, as discussed herein below, such as, for example, one of the liquid chromatography methodologies including HPLC and DHPLC. Typically, MS detection does not require the addition of a tag or label to the small nucleic acid fragment. Instead, the nucleic acid fragment can be identified directly in the mass spectrometer.

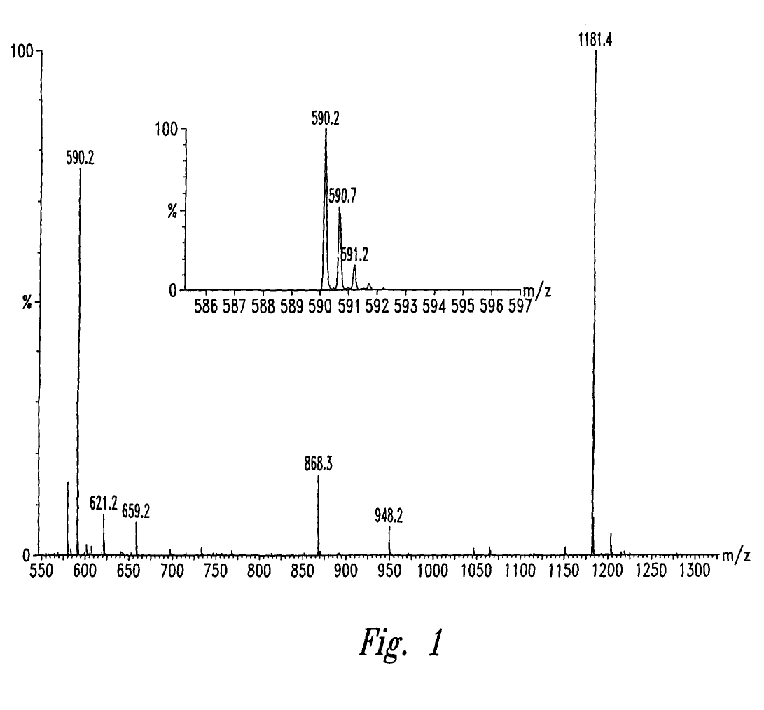

As disclosed herein in Examples 1 and 2 and FIGS. 1-9, MS may be particularly suitable to the detection of small nucleic acid fragments from as small as 1 nucleic acid to as large as several hundred nucleotides. More preferable are fragments of 1 to 50 nucleotides, still more preferable are fragments of from 1 to 14 nucleotides.

Sensitivities may be achieved to at least to 1 amu. The smallest mass difference in nucleic acid bases is between adenine and thymidine, which is 9 Daltons.

Particularly preferred MS methodologies employ Liquid Chromatography-Time-of-Flight Mass Spectrometry (LC-TOF-MS). LC-TOF-MS is composed of an orthogonal acceleration Time-of-Flight (TOF) MS detector for atmospheric pressure ionization (API) analysis using electrospray (ES) or atmospheric pressure chemical ionization (APCI). LC-TOF-MS provides high mass resolution (5000 FWHM), high mass measurement accuracy (to within 5 ppm) and very good sensitivity (ability to detect picomolar amount of DNA polymer) compared to scanning quadrupole instruments. TOF instruments are generally more sensitive than quadrupoles, but correspondingly more expensive.

LC-TOF-MS has a more efficient duty cycle since the current instruments can sequentially analyze one mass at a time while rejecting all others (this is referred to as single ion monitoring (SIM)). LC-TOF-MS samples all of the ions passing into the TOF analyzer at the same time. This results in higher sensitivity and provides quantitative data which improves the sensitivity between 10 and 100 fold. Enhanced resolution (5000 FWHM) and mass measurement accuracy of better than 5 ppm imply that differences between nucleosides as small as 9 amu (Daltons) can be accurately measured. The TOF mass analyzer performs very high frequency sampling (10 spectra/sec) of all ions simultaneously across the full mass range of interest. The duty cycle of the LC-TOF-MS allows high sensitivity spectra to be recorded in quick succession making the instrument compatible with more efficient separation techniques such as narrow bore LC, capillary chromatography (CE) and capillary electrochromatography (CEC). The ions are pulsed into the analyzer, effectively taking a “snapshot” of the ions present at any time.

In the first stage the ES or APCI, aerosol spray is directed perpendicularly past the sampling cone, which is displaced from the central axis of the instrument. Ions are extracted orthogonally from the spray into the sampling cone aperture leaving large droplets, involatile materials, particulates and other unwanted components to collect in the vent port that is protected with an exchangeable liner. The second orthogonal step enables the volume of gas (and ions) sampled from atmosphere to be increased compared with conventional API sources. Gas at atmospheric pressure sampled through an aperture into a partial vacuum forms a freely expanding jet, which represents a region of high performance compared to the surrounding vacuum. When this jet is directed into the second aperture of a conventional API interface it increases the flow of gas through the second aperture. Maintaining a suitable vacuum in the MS-TOF therefore places a restriction on the maximum diameter of the apertures in such an LC interface. Ions in the partial vacuum of the ion block are extracted electrostatically into the hexapole ion bridge which efficiently transports ions to the analyzer.

The coupling of the TOF mass analyzers with MUX-technology allows the connection of up to 8 HPLC columns in parallel to a single LC-TOF-MS. (Micromass, Manchester UK). A multiplexed electrospray (ESI) interface is used for on-line LC-MS utilizing an indexed stepper motor to sequentially sample from up to 8 HPLC columns or liquid inlets operated in parallel.

Use of LC-TOF-MS is generally preferred over use of MALDI-TOF because LC-TOF-MS is a quantitative method for analysis of the molecular weight of polymers. LC-TOF-MS does not fragment the polymers and it employs a very gentle ionization process compared to matrix-assisted-lazer-desorption-ionization (MALDI). Because every MALDI blast is different, the ionization is not quantitative. LC-TOF-MS does, however, produce different m/z values for polymers, but, as disclosed in Example 1 and FIGS. 1-9, this property provides the additional advantage of reducing background and providing complementary information.

Tandem MS or MS/MS is used for structure determination of molecular ions or fragments. In Tandem MS, the ion of interest is selected with the first analyzer (MS-1), collided with inert gas atoms in a collision cell, and the fragments generated by the collision are separated by a second analyzer (MS-2). In Ion Trap and Fourier transform experiments, the analyses are carried out in one analyzer, and the various events are separated in time, not in space. The information can be used to sequence peptides and small DNA/RNA oligomers.

Exact mass measurements, sometimes referred to as “high-resolution measurements,” are used for elemental-composition determination of the sample molecular ion or an ionic fragment. The basis of the method is that each element has a unique mass defect (deviation from the integer mass). The measurement is carried out by scanning with an internal calibrant (in EI or CI mode) or by peak matching (in FAB mode). The elemental composition is determined by comparing the masses of many possible compositions to the measured one. The method is very reliable for samples having masses up to 800 Da. At higher masses, higher precision or knowledge of expected composition are required to determine the elemental composition unambiguously.

Electron ionization (EI) is widely used in mass spectrometry for relatively volatile samples that are insensitive to heat and have relatively low molecular weight. The spectra, usually containing many fragment-ion peaks, are useful for structural characterization and identification. Small impurities in the sample are easy to detect. Chemical ionization (CI) is applied to similar samples; it is used to enhance the abundance of the molecular ion. For both ionization methods, the molecular weight range is 50 to 800 Da. In rare cases it is possible to analyze samples of higher molecular weight. Accuracy of the mass measurement at low resolving power is ±0.1 Dalton and in the high resolution mode, ±5 ppm.

Fast atom bombardment ionization (FAB or sometimes called liquid secondary ionization MS, LSIMS) is a softer ionization method than EI. The spectrum often contains peaks from the matrix, which is necessary for ionization, a few fragments and a peak for a protonated or deprotonated sample molecule. FAB is used to obtain the molecular weight of sensitive, nonvolatile compounds. The method is prone to suppression effects by small impurities. The molecular weight range is 100 to 4000 Da. Exact mass measurement is usually done by peak matching. The accuracy of the mass is the same as obtained in EI, CI.

Matrix-assisted laser desorption (MALDI) has been used to determine the molecular weight of peptides, proteins, oligonucleotides, and other compounds of biological origin as well as of small synthetic polymers. The amount of sample needed is very low (pmoles or less). The analysis can be performed in the linear mode (high mass, low resolution) up to a molecular weight of m/z 300,000 (in rare cases) or reflectron mode (lower mass, higher resolution) up to a molecular weight of 10,000. The analysis is relatively insensitive to contaminants, and accordingly a purification step is not necessarily a part of the characterization process when characterization includes MS. Mass accuracy (0.1 to 0.01%) is not as high as for other mass spectrometry methods. Recent development in Delayed Extraction TOF allows higher resolving power and mass accuracy.

Electrospray ionization (ESI) allows production of molecular ions directly from samples in solution. It can be used for small and large molecular-weight biopolymers (peptides, proteins, carbohydrates, and DNA fragments), and lipids. Unlike MALDI, which is pulsed, it is a continuous ionization method that is suitable for using as an interface with HPLC or capillary electrophoresis. Multiply charged ions are usually produced. ESI should be considered a complement to MALDI. The sample must be soluble, stable in solution, polar, and relatively clean (free of nonvolatile buffers, detergents, salts, etc.).

Electron-capture (sometimes called negative ion chemical ionization or NICI) is used for molecules containing halogens, NO₂, CN, etc, and it usually requires that the analyte be derivatized to contain highly electron-capturing moieties (e.g., fluorine atoms or nitrobenzyl groups). Such moieties are generally inserted into the target analyte after isolation and before mass spectrometric analysis. The sensitivity of NICI analyses is generally two to three orders of magnitude greater than that of PCI or EI analyses. Little fragmentation occurs during NICI.

### b. Indirect Characterization

In some embodiments of the present invention, it may be advantageous to add one or more detectable labels to a short nucleic acid fragment or the reaction product thereof (e.g., a portion or the whole complementary strand of the short nucleic acid fragment). Such labels facilitate the characterization of the fragment and thereby the identification of nucleotide(s) of interest and/or genetic variations within the fragment.

Tables 2 and 3 summarize exemplary labels and detectors, respectively, that are generally suitable for use in methodologies for detecting small nucleic acid fragments.

TABLE 2
Labels Suitable for use in Methodologies for
Detecting Small Nucleic Acid Fragments

| Tagging Technologies | Attributes |
|---|---|
| Fluorophores | Multi-color, overlapping emission spectra, inexpensive |
| | detectors |
| FRET | High sensitivity |
| Fluorescent quenching | Homogenous assay formats |
| Time-resolved fluorescence | Low background |
| Colloidal gold | Good sensitivity |
| Mass Tags (CMSTs) | High level of multiplexing |
| Mass Tags (Electrophore) | High level of multiplexing |
| Radiolabels | Excellent sensitivity |
| Chemiluminescence | Excellent sensitivity |
| Colorimetric | Inexpensive |
| Assay product = “Tag” | Accurate, inexpensive, direct |

TABLE 3
Detectors Suitable for use in Methodologies for
Detecting Small Nucleic Acid Fragments

| Detector | Attributes |
|---|---|
| Film | Inexpensive |
| Scintillation Counter | Reliable, sensitive |
| Fluorescent plate reader | Reliable, inexpensive, sensitive, multicolor |
| Fluorescence Polarization | Permits homogeneous assay formats, some instruments |
| | very sensitive. |
| Time-resolved fluorescence | Low background, sensitive |
| Fluorescent-monitoring of | Useful information on the process of PCR |
| PCR | |
| ABI-377 | Reliable |
| Capillary Instrument | High throughput, expensive |
| Chemiluminescence plate | Reliable, sensitive |
| reader | |
| CCD | Versatile, sensitive |
| Quadrupole MS | Wide spectral range, quantitative |
| GC/MS | |
| Maldi-TOF | Wide spectral range, not quantitative |
| Plate Reader (colorimetric | Reliable, inexpensive, sensitive |
| assays) | |
| Cell Sorter | High throughput |
| Light Microscopy (Confocal) | Excellent sensitivity |
| Electron mic oscopy | Sensitivity |
| Amphoteric device | Ability to multiplex |
| DHPLC (HPLC/UV) | Reliable, relatively inexpensive |
| HPLC/Fluorescence | Reliable, sensitive, relatively inexpensive |
| Text scanner | Very inexpensive, make your own assay |
| UV box (for stains) | Very inexpensive |

Detectors for these tags and labels are available in generic and non-generic instruments. The generic instruments are the plate readers that usually read micro-plates in 96-well or 384-well formats, and are capable of reading multiple colors (4-6 fluorescent tags). These instruments can be found in customized versions to perform more specialized measurements like time-resolyed-fluorescence (TFR) or fluorescence polarization. The detectors for PAGE sequencing and bundled capillary instruments are highly dedicated and non-generic. The generic mass spectrometers MALDI-TOF, electrospray-TOF and APCI-quadrupole (and combinations thereof including ion-trap instruments) are opened-ended instruments with versatility. Suitable software packages have been developed for combinatorial chemistry applications. Scintillation counters are dedicated in that they need to be used with radioisotopes, but can accommodate a wide range of assays formats.

The following is exemplary indirect characterization methodologies. However, the present invention is not limited to these examples. Any techniques known in the art suitable for characterizing small nucleic acid fragments and thereby determining the identity of nucleotide(s) at a defined location may be used in the present invention.

### i. i. Sequencing

In one aspect of the invention, a nucleic acid fragment (i.e., a digestion product described above) is characterized by performing a complete nucleotide sequence analysis. Many techniques are known in the art for identifying each of the bases in a nucleic acid fragment, so as to obtain base sequence information. For instance, two different DNA sequencing methodologies that were developed in 1977, and are commonly known as “Sanger sequencing” and “Maxam Gilbert sequencing,” among other names, are still in wide use today and are well known to those of ordinary skill in the art. See, e.g., Sanger, Proc. Natl. Acad. Sci. (USA) 74:5463, 1977) and Maxam and Gilbert, Proc. Natl. Acad. Sci. (USA) 74:560, 1977). Both methods produce populations of shorter fragments that begin from a particular point and terminate in every base that is found in the nucleic acid fragment that is to be sequenced. The shorter nucleic acid fragments are separated by polyacrylamide gel electrophoresis and the order of the DNA bases (adenine, cytosine, thymine, guanine; also known as A, C, T, G, respectively) is read from a autoradiograph of the gel.

Automated DNA sequencing methods may also be used. Such methods are in wide-spread commercial use to sequence both long and short nucleic acid molecules. In one approach, these methods use fluorescent-labeled primers or ddNTP-terminators instead of radiolabeled components. Robotic components can utilize polymerase chain reaction (PCR) technology which has lead to the development of linear amplification strategies. Current commercial sequencing allows all 4 dideoxy-terminator reactions to be run on a single lane. Each dideoxy-terminator reaction is represented by a unique fluorescent primer (one fluorophore for each base type: A, T, C, G). Only one template DNA (i.e., DNA sample) is represented per lane. Current gels permit the simultaneous electrophoresis of up to 64 samples in 64 different lanes. Different ddNTP-terminated fragments are detected by the irradiation of the gel lane by light followed by detection of emitted light from the fluorophore. Each electrophoresis step is about 4-6 hours long. Each electrophoresis separation resolves about 400-600 nucleotides (nt), therefore, about 6000 nt can be sequenced per hour per sequencer.

Gilbert has described an automated DNA sequencer (EPA, 92108678.2) that consists of an oligomer synthesizer, an array on a membrane, a detector which detects hybridization and a central computer. The synthesizer synthesizes and labels multiple oligomers of arbitrary predicted sequence. The oligomers are used to probe immobilized DNA on membranes. The detector identifies hybridization patterns and then sends those patterns to a central computer which constructs a sequence and then predicts the sequence of the next round of synthesis of oligomers. Through an iterative process, a DNA sequence can be obtained in an automated fashion. This approach may be used to characterize a short nucleic acid fragment (either double or, more commonly single stranded) according to the present invention.

The use of mass spectrometry for the study of monomeric constituents of nucleic acids has also been described (ignite, In Biochemical Applications of Mass Spectrometry, Waller and Dermer (eds.), Wiley-Interscience, Chapter 16, p. 527, 1972). Briefly, for larger oligomers, significant early success was obtained by plasma desorption for protected synthetic oligonucleotides up to 14 bases long, and for unprotected oligos up to 4 bases in length. As with proteins, the applicability of ESI-MS to oligonucleotides has been demonstrated (Covey et al., Rapid Comm. in Mass Spec. 2:249-256, 1988). These species are ionized in solution, with the charge residing at the acidic bridging phosphodiester and/or terminal phosphate moieties, and yield in the gas phase multiple charged molecular anions, in addition to sodium adducts. These approaches to nucleic acid characterization may be used according to the present invention.

Sequencing nucleic acids with <100 bases by the common enzymatic ddNTP technique is more complicated than it is for larger nucleic acid templates, so that chemical degradation is sometimes employed. However, the chemical decomposition method requires about 50 pmol of radioactive ³²P end-labeled material, 6 chemical steps, electrophoretic separation, and film exposure. For small oligonucleotides (<14 nts), as may need to be characterized according to the present invention, the combination of electrospray ionization (ESI) and Fourier transform (FT) mass spectrometry (MS) is far faster and more sensitive, and is a preferred method for the present invention. Dissociation products of multiply-charged ions measured at high (105) resolving power represent consecutive backbone cleavages providing the full sequence in less than one minute on sub-picomole quantity of sample (Little et al., J. Am. Chem. Soc. 116:4893, 1994). For molecular weight measurements, ESI/MS has been extended to larger fragments (Potier et al., Nuc. Acids Res. 22:3895, 1994). ESI/FTMS appears to be a valuable complement to classical methods for sequencing and pinpoint mutations in nucleotides as large as 100-mers. Spectral data have recently been obtained loading 3×10-13 mol of a 50-mer using a more sensitive ESI source (Valaskovic, Anal. Chem. 68:259, 1995).

Other methods for obtaining complete, or near complete base sequence information for a nucleic acid molecule are described in the following references: Brennen et al. (Biol. Mass Spec., New York, Elsevier, p. 219, 1990); U.S. Pat. No. 5,403,708); PCT Patent Application No. PCT/US94/02938; and PCT Patent Application No. PCT/US94/11918.**

FP is based on the property that when a fluorescent molecule is excited by plane-polarized light, it emits polarized fluorescent light into a fixed plane if the tagged-molecules do not significantly rotate between excitation and emission. If the molecule is small enough and rotates and tumbles in space, however, fluorescence polarization is not observed fully by the detector.

The fluorescence polarization of a molecule is proportional to the molecule's rotational relaxation time (usually the time it takes to rotate through an angle of 68.5°), which is related to properties of the solution such as the viscosity, temperature, and molecular volume of the analyte or biomolecule. Therefore, if the viscosity and temperature are held constant, fluorescence polarization is directly proportional to molecular volume, which, in turn, is directly proportional to molecular weight. Larger tagged molecules rotate and tumble slowly in space and, accordingly, fluorescence polarization values can be obtained. In contrast, smaller molecules rotate and tumble faster and fluorescence polarization cannot be measured.

The present invention exploits the property that small nucleic acid fragments tumble slowly in solution and, consequently, are amenable to detection by fluorescence polarization. In contrast, fluorescent-tagged-nucleoside triphosphates (including dideoxyribonucleoside triphosphates (ddNTPs), deoxyribonucleoside triphosphates (DNTPs) and ribonucleoside triphosphates (rNTPs)) are small and rotate rapidly in solution. Thus, absent incorporation into a small nucleic acid fragment, fluorescent-tagged-nucleoside triphosphates are undetectable by fluorescence polarization.

By the methodology of the present invention, fluorescent-tagged-nucleoside triphosphates are made detectable by their incorporation into small nucleic acid fragments. The methods described herein may be performed homogenously in a single vessel and may employ a nucleotide fill-in reaction wherein a labeled deoxynucleoside or ribonucleoside is incorporated into the end of the small nucleic acid fragment. More specifically, the target nucleic acid of interest may be amplified from a population of nucleic acids from a biological sample and the resulting amplicon incubated with a RE, e.g., EcoN I thereby generating two fragments with recessed 3′ ends.

In some embodiments, the nucleotide of interest (ie., a genetic variation) or its complement is located within a 5′ overhang (i.e., oligonulceotides (b) and (c) of FIG. 21, panel (B)), and fluorescent-labeled nucleotide(s) are incorporated into the complement of the nucleic acid fragment containing the nucleotide of interest or its complement nucleotide by a fill-in reaction. Preferably, the fill-in reaction is performed in the presence of allele-specific dye-labeled ddNTPs and a commercially available modified Taq DNA polymerase. An alternative preferable method to incorporate fluorescent label is to use a RNA polymerase to fill in a recessed 3′ end with fluorescent-labeled rNTPs. Such a fill-in reaction eliminates the need of using alkaline phosphatase to dephosphorylate all the existing dNTPs. In standard primer extension assays, alkaline phosphatase must be used to dephosphorylate all the existing dNTPs to permit the efficient incorporation of the ddNTPs. By using an RNA polymerase that has strict requirements for rNTPs and the inability to use dNTPs or ddNTPs, the need for a de-phosphorylation step is abolished.

The fill-in reaction places the fluorescent-labeled nucleoside specific for the allele of the template, increasing about 20-fold the molecular weight of the fluorophore. At the end of the reaction, the fluorescence polarization of the resulting fluorescent-label oligonucleotides, as well as remaining fluorescent-labeled nucleoside triphosphates, in the reaction mixture is analyzed directly without separation or purification.

Currently, fluorometers and more than 50 fluorescence polarization immunoassays (FPIAs) are commercially available, many of which are routinely used in clinical laboratories for the measurement of therapeutics, metabolites, and drugs of abuse in biological fluids. (See, e.g., Checovich et al., Nature 375:254-256 (1995); published erratum appears in Nature 375:520 (1995) both or which are incorporated herein by reference in their entirety).

Various fluorescent dyes or probes may be employed in the present invention. Fluorescent dyes are identified and quantified most directly by their absorption and fluorescence emission wavelengths and intensities. Emission spectra (fluorescence and phosphorescence) are much more sensitive and specific than absorption spectra. Other photophysical characteristics (like fluorescence anisotropy) are less widely used. The useful intensity parameters are quantum yield (QY) for fluorescence, and the molar extinction coefficient (ε) for absorption. QY is a measure of the total photon emission over the entire fluorescence spectral profile and the value of ε is specified at a given wavelength (usually the absorption maximum of the probe). A narrow optical bandwidth (<25 nm) is usually used for fluorescence excitation (via absorption), whereas the fluorescence detection bandwidth is more variable, ranging from full spectrum for maximal sensitivity to narrow band (˜20 mm) for maximal resolution. Fluorescence intensity per probe molecule is proportional to the product of ε and QY. Commercially important and exemplary fluorochromes that are widely used are fluorescein, tetramethylrhodamine, lissamine, Texas Red and BODIPYs.

Fluorescent labels are now commonly used for the detection of small nucleic acid fragments that have been separated by capillary electrophoresis (CE) or high-performance liquid chromatography (HPLC). One group of labels that may be employed for this purpose are those based on near-infrared (near-IR) fluorescent dyes. In aqueous solution, these types of tags have a maximum absorption of light at >680 nm, followed by the emission of fluorescence at near-IR wavelengths (emission maximum, >700 nm). One advantage of using this type of fluorescence for detection is that it occurs in a spectral region where there is relatively little absorption or emission due to other compounds that might be present in biological samples. This, plus the fact that most near IR probes can be excited with commercially available lasers, provides this approach with low background signals and limits of detection that extend into the attomole range.

One of the limitations of fluorescence is the phenomenon of autofluorescence. An elegant method to avoid autofluorescence is to employ fluorochromes that possess significantly longer delay times to emission (see Fernandes for review). These fluorochromes are usually luminescent metal chelates that are attached at the 5′-end of an ODN probe or primer.

Terbium deoxynucleoside triphosphates are available that allow the incorporation of time-resolved fluorochromes into “natural” nucleic acids. These probes have the advantage of the large Stokes shift, narrow emission bands and long lifetimes. Time-resolved fluorescence spectroscopy is particularly useful in structural biology and is used to monitor molecular interactions and motions that occur in the picosecond-nanosecond time range. Time-resolved fluorescence spectroscopy is beginning to dominate the analysis of biomolecular structure and dynamics.

Deoxyribonucleoside analogs that may be incorporated into a small nucleic acid fragment of the present invention, to thereby afford an effective characterization means for the small nucleic acid, include but are not limited to: Fluorescein-12-dUTP, Coumarin-5-dUTP, Tetramethylrhodamine-6-dUTP, Texas Red®-5-dUTP, Napthofluorescein-5-dUTP, Fluorescein Chlorotriazinyl-4-dUTP, Pyrene-8-dUTP, Diethylaminocoumarin-5-dUTP,Cyanine 3-dUTP, Cyanine 5-dUTP, Coumarin-5-dCTP, Fluorescein-12-dCTP, Tetramethylrhodamine-6-dCTP, Texas Red®-5-dCTP, Lissamine™-5-dCTP, Napthofluorescein-5-dCTP, Fluorescein Chlorotriazinyl-4-dCTP, Pyrene-8-dCTP, Diethylaminocoumarin-5-dCTP, Cyanine 3-dCTP, Cyanine 5-dCTP, Coumarin-5-dATP, Diethylaminocoumarin-5-dATP, Fluorescein-12-dATP, Fluorescein Chlorotriazinyl-4-dATP, Lissamine™-5-dATP, Napthofluorescein-5-dATP, Pyrene-8-dATP, Tetramethylrhodamine-6-dATP, Texas Red®-5-dATP, Cyanine 3-dATP, Cyanine 5-dATP, Coumarin-5-dGTP, Fluorescein-12-dGTP, Tetramethylrhodamine-6-dGTP, Texas Red®-5-dGTP, Lissamine™-5-dGTP.

Ribonucleoside analogs include but are not limited to: Fluorescein-12-UTP, Coumarin-5-UTP, Tetramethylrhodamine-6-UTP, Texas Red®-5-UTP, Lissamine™-5-UTP, Napthofluorescein-5-UTP, Fluorescein Chlorotriazinyl-4-UTP, Pyrene-8-UTP, Cyanine 3-UTP, Cyanine 5-UTP, Coumarin-5-CTP, Fluorescein-12-CTP, Tetramethylrhodamine-6-CTP, Texas Red®-5-CTP. Lissamine™-5-CTP, Napthofluorescein-5-CTP, Fluorescein Chlorotriazinyl-4-CTP, Pyrene-8-CTP, Cyanine 3-CTP, Cyanine 5-CTP, Coumarin-5-ATP, Fluorescein-12-ATP, Tetramethylrhodamine-6-ATP, Texas Red®-5-ATP, Lissamine™-5-ATP, Coumarin-5-GTP, Fluorescein-12-GTP, Tetramethylrhodamine-6-GTP, Texas Red®-5-GTP, Lissamine™-5-GTP.

Dideoxy analogs include but are not limited to: Fluorescein-12-ddUTP, FAM-ddUTP, ROX-ddUTP, R6G-ddUTP, TAMRA-ddUTP, JOE-ddUTP, R110-ddUTP, Fluorescein-12-ddCTP, FAM-ddCTP, ROX-ddCTP, R6G-ddCTP, TAMRA-ddCTP, JOE-ddCTP, R110-ddCTP, Fluorescein-12-ddGTP, FAM-ddGTP, ROX-ddGTP, R6G-ddGTP, TAMRA-ddGTP, JOE-ddGTP, R110-ddGTP, Fluorescein-12-ddATP, FAM-ddATP, ROX-ddATP, R6G-ddATP, TAMRA-ddATP, JOE-ddATP, R110-ddATP.

All of the above analogs can be radiolabeled with ³H, deuterium, 32P, ¹⁴C, ³⁵S and other radioisotopes.

Analogs can also be un-natural nucleoside analogs including, but not limited to, the following: 8-Bromo-2′-deoxyadenosine-TTP, 8-Oxo-2′-deoxyadenosine, Etheno-2′-deoxyadenosine-TTP, Etheno-2′-deoxyadenosine-TTP, N⁶-Methyl-2′deoxyadenosine-TTP, 2,6-Diaminopurine-2′-deoxyriboside-TTP, 8-Bromo-2′-deoxyguanosine-TTP, 7-Deaza-2′-deoxyguanosine-TTP, 2′-Deoxyisoguanosine-TTP, -Oxo-2′-deoxyguanosine-TTP, O⁶-Methyl-2′-deoxyguanosine-TTP, S⁶-DNP-2′-deoxythioguanosine-TTP, 3-Nitropyrrole-2′-deoxyriboside-TTP, 5-Propynyl-2′-deoxyuridine-TTP, 5-Fluoro-2′-deoxyuridine-TTP, 2′-deoxyuridine-TTP, 5-Bromo-2′-deoxyuridine-TTP, 5-Iodo-2′-deoxyuridine-TTP, 4-Triazolyl-2′-deoxyuridine-TTP.

Besides the use of determining the identity of nucleotides of interest in target nucleic acids, the above method may also be useful for determining whether the organism from which the targets are isolated contains homozygous wild type alleles, two or more variant alleles, or both wild type and variant alleles (i.e., heterozygous alleles). Such determination is due to the fact that different fluorophores may be used to label nucleotide specific to a wild type allele and various variant alleles. For instance, the detection of the incorporation of both wild type-specific and variant allele-specific fluorescent labeled nucleosides would indicate the organism contain heterozygous alleles. In addition, the present method may further be used in measuring allelic frequency by quantifying different types of fluorescent-labeled nucleosides incorporated when the target nucleic acids are from a selected population of organisms.

### ii. Fluid Handling

As used herein, the term “Fluid Handling” refers to those assays that are array (including microtiter-plate) based and use fluorescence, fluorescence-polarization, luminescence, radioactivity (scintillation counters), or colorimetric readouts. Fluid handling may be useful when the characterization method employs modification of the short nucleic acid fragment, e.g., when a tag or label is incorporated into the short nucleic acid fragment. These assays can be amplified by the use of enzymes such as horseradish peroxidase or alkaline phosphatase that can generate soluble or insoluble colorimetric products from soluble substrates or sensitive luminescent products. These assays have large dynamic ranges (6-8 logs) and can be made robust. Fluid Handling using microplates scales well and has been partially miniaturized by the use of 384-well plates. Fluid Handling is especially compatible with commercial robotics and readout systems such as fluorometers, and plate readers. The data is easy to archive and manipulate.

Besides microplates, other types of ODNP arrays may be used with the present method. In a preferred embodiment, labeled or tagged nucleoside(s) are incorporated onto an immobilized ODNP (i.e., oligonucleotide (a) in FIG. 21) by filling in a 3′ recessed terminus resulting from digestion with a RE (e.g., EcoN I) in a way similar to that described above in the “Fluorescence Polarization” section. The only difference between the two filling-in reactions is that for immobilized ODNPs, even if DNA polymerase is used for the filling-in reaction, dephosphorylation of excessive labeled dNTPs prior to the reaction is not necessary. The elimination of the need for dephosphorylation is due to the fact that the excessive dNTPs may be removed by a simple wash of the array.

The means of detection for the labeled ODNPs of an array will be selected in combination with the type of labels and arrays used and various other considerations. For instance, for fluorescent labeled ODNPs of a microarray, a detection device may include a microscope and a monochromatic or polychromatic light source for directing light at the substrate of the array. A photon counter detects fluorescence from the substrate, while an x-y translation stage varies the location of the substrate. Exemplary devices are described in U.S. Pat. Nos. 6,141,096, 5,143,854, and 6,045,996. These patents are incorporated herein by reference in their entirety.

Similar to fluorescence polarization, fluid handling may also be useful for determining whether the organism from which target nucleic acids are isolated contains homozygous wild type alleles, two or more variant alleles, or both wild type and variant alleles (i.e., heterozygous alleles). In addition, the method may further be used in measuring allelic frequency by quantifying different types of labeled nucleosides incorporated when the target nucleic acids are from a selected population of organisms.

### c. Fractionation Methodologies

According to the present invention, the small nucleic acid fragment(s) may, optionally, undergo a step of fractionation prior to a step of detection. The fractionation step may simply remove undesired impurities from the small fragment of interest, to allow more convenient and/or more accurate characterization of the fragment. This type of fractionation step may be referred to as purification. Alternatively, or in addition, the fractionation may separate nucleic acids from one another (such as in chromatography) and the detection technique is simply determining whether the nucleic acid is, or is not, present at a particular time and space (e.g., using ultraviolet detection to determine whether a nucleic acid is eluting from a chromatography column).

Thus, depending on the particular detection methodology employed, it may be advantageous to couple a detection methodology with one or more methodologies for the fractionation of small nucleic acid fragments. As discussed below, such fractionation methodologies include, but are not limited to, electrophoresis including polyacrylamide or agarose gel electrophoresis and capillary electrophoresis, and liquid chromatography (LC) including high pressure liquid chromatography (HPLC) and denaturing high pressure liquid chromatography (DHPLC).

### i. Gel Electrophoresis

As used herein, the term “electrophoresis” refers generally to those separation techniques based on the mobility of nucleic acid in an electric field. Negatively charged nucleic acid migrates towards a positive electrode and positively charged nucleic acid migrates toward a negative electrode. Charged species have different migration rates depending on their total charge, size, and shape, and can therefore be separated.

An electrophoresis apparatus consists of a high-voltage power supply, electrodes, buffer, and a support for the buffer such as a polyacrylamide gel, or a capillary tube. Open capillary tubes are used for many types of samples and the other gel supports are usually used for biological samples such as protein mixtures or nucleic acid fragments.

The most powerful separation method for nucleic acid fragments is PAGE, generally in a slab gel format. The major limitation of the current technology is the relatively long time required in performing the gel electrophoresis of nucleic acid fragments produced in sequence reactions. An increased magnitude (10-fold) can be achieved with the use of capillary electrophoresis which utilize ultrathin gels.

Capillary electrophoresis (CE) in its various forms, including free solution, isotachophoresis, isoelectric focusing, PAGE, and micellar electrokinetic “chromatography,” is a suitable technology for the rapid, high resolution separation of very small sample volumes of complex mixtures. In combination with the inherent sensitivity and selectivity of mass spectrometry (CE-MS; see below), CE is a potentially powerful technique for bioanalysis. In the methodology disclosed herein, the interfacing of these two methods provides superior DNA sequencing methods that are superior to the current rate methods of sequencing.

By alternate embodiments, CE may be employed in conjunction with electrospray ionization (ESI) flow rates. The combination of both capillary zone electrophoresis (CZE) and capillary isotachophoresis with quadrapole mass spectrometers based upon ESI have been described. (Olivares et al., Anal. Chem. 59:1230 (1987); Smith et al., Anal. Chem. 60:436 (1988); Loo et al., Anal. Chem. 179:404 (1989); Edmonds et al., J. Chroma. 474:21 (1989); Loo et al., J. Microcolumn Sep. 1:223 (1989); Lee et al., J. Chromatog. 458:313 (1988); Smith et al., J. Chromatog. 480:211 (1989); Grese et al., J. Am. Chem. Soc. 111:2835 (1989) each of which is incorporated herein by reference in its entirety). Small peptides are easily amenable to CZE analysis with good (femtomole) sensitivity.

Polyacrylamide gels, such as those discussed above, may be applied to CE methodologies. Remarkable plate numbers per meter have been achieved with cross-linked polyacrylamide. (See, e.g., Cohen et al., Proc. Natl. Acad. Sci., USA 85:9660 (1988) reporting 10⁺⁷ plates per meter). Such CE columns as described can be employed for nucleic acid (particularly DNA) sequencing. The CE methodology is in principle 25 times faster than slab gel electrophoresis in a standard sequencer. For example, about 300 bases can be read per hour. The separation speed is limited in slab gel electrophoresis by the magnitude of the electric field that can be applied to the gel without excessive heat production. Therefore, the greater speed of CE is achieved through the use of higher field strengths (300 V/cm in CE versus 10 V/cm in slab gel electrophoresis). The capillary format reduces the amperage and thus power and the resultant heat generation.

In alternative embodiments, multiple capillaries may be used in parallel to increase throughput and may be used in conjunction with high throughput sequencing. (Smith et al., Nuc. Acids. Res. 18:4417 (1990); Mathies et al., Nature 359:167 (1992); Huang et al., Anal. Chem. 64:967 (1992); Huang et al., Anal. Chem. 64:2149 (1992)). The major disadvantage of capillary electrophoresis is the limited volume of sample that can be loaded onto the capillary. This limitation may be circumvented by concentrating large sample volumes prior to loading the capillary with the accompanying benefit of >10-fold enhancement in detection.

The most popular method of preconcentration in CE is sample stacking. (Chien et al., Anal. Chem. 64:489A (1992)). Sample stacking depends on the matrix difference (i.e., pH and ionic strength) between the sample buffer and the capillary buffer, so that the electric field across the sample zone is more than in the capillary region. In sample stacking, a large volume of sample in a low concentration buffer is introduced for preconcentration at the head of the capillary column. The capillary is filled with a buffer of the same composition, but at higher concentration. When the sample ions reach the capillary buffer and the lower electric field, they stack into a concentrated zone. Sample stacking has increased detectability by 1-3 orders of magnitude.

Alternatively, preconcentration may be achieved by applying isotachophoresis (ITP) prior to the free zone CE separation of analytes. ITP is an electrophoretic technique that allows microliter volumes of sample to be loaded onto the capillary, in contrast to the low nL injection volumes typically associated with CE. This technique relies on inserting the sample between two buffers (leading and trailing electrolytes) of higher and lower mobility followed by the analyte. The technique is inherently a concentration technique, where the analytes concentrate into pure zones migrating with the same speed. The technique is currently less popular than the stacking methods described above because of the need for several choices of leading and trailing electrolytes, and the ability to separate only cationic or anionic species during a separation process.

Central to the nucleic acid sequencing process is the remarkably selective electrophoretic separation that may be achieved with nucleic acid and/or ODN fragments. Separations are routinely achieved with fragments differing in sequence by only a single nucleotide. This methodology is suitable for separations of fragments up to 1000 bp in length. A further advantage of sequencing with cleavable tags is that there is no requirement to use a slab gel format when nucleic acid fragments are separated by PAGE. Since numerousx samples are combined (4 to 2000) there is no need to run samples in parallel as is the case with current dye-primer or dye-terminator methods (i.e., ABI 373 sequencer). Since there is no reason to run parallel lanes, there is no reason to use a slab gel. Therefore, one can employ a tube gel format for the electrophoretic separation method. It has been shown that considerable advantage is gained when a tube gel format is used in place of a slab gel format. (Grossman et al., Genet. Anal. Tech. Appl. 9:9 (1992)). This is due to the greater ability to dissipate Joule heat in a tube format compared to a slab gel which results in faster run times (by 50%), and much higher resolution of high molecular weight nucleic acid fragments (greater than 1000 nt). Long reads are critical in genomic sequencing. Therefore, the use of cleavable tags in sequencing has the additional advantage of allowing the user to employ the most efficient and sensitive nucleic acid separation method that also possesses the highest resolution.

As discussed above, CE is a powerful method for nucleic acid sequencing, particularly DNA sequencing, forensic analysis, PCR product analysis and restriction fragment sizing. CE is faster than traditional slab PAGE since with capillary gels a higher 6+potential field can be applied, but has the drawback of allowing only one sample to be processed per gel. Thus, by alternative embodiments, micro-fabricated devices (MFDs) are employed to combine the faster separations times of CE with the ability to analyze multiple samples in parallel.

MFDs permit an increase in information density in electrophoresis by miniaturizing the lane dimension to about 100 micrometers. The current density of capillary arrays is limited to the outside diameter of the capillary tube. Microfabrication of channels produces a higher density of arrays. Microfabrication also permits physical assemblies not possible with glass fibers and links the channels directly to other devices on a chip. A gas chromatograph and a liquid chromatograph have been fabricated on silicon chips, but these devices have not been widely used. (Terry et al., IEEE Trans. Electron Device ED-26:1880 (1979) and Manz et al., Sens. Actuators B1:249 (1990)). Several groups have reported separating fluorescent dyes and amino acids on MFDs. (Manz et al., J. Chromatography 593:253 (1992); Effenhauser et al., Anal. Chem. 65:2637 (1993)).

Photolithography and chemical etching can be used to make large numbers of separation channels on glass substrates. The channels are filled with hydroxyethyl cellulose (HEC) separation matrices. DNA restriction fragments could be separated in as little as two minutes. (Woolley et al., Proc. Natl. Acad. Sci. 91:11348 (1994))

### ii. Liquid Chromatography (LC)

Liquid chromatography, including HPLC and DHPLC, may be used in conjunction with one of the detection methodologies discussed above such as, for example, fluorescence polarization, mass spectrometry and/or electron ionization. Alternatively LC, HPLC and/or DHPLC may be utilized in conjunction with a UV detection methodology. Regardless of the detection methodology employed, a fractionation step provides the separation of complex mixtures of non-volatile compounds prior to detection.

LC may be used for compounds that have a high molecular weight or are too sensitive to heat to be analyzed by GC. The most common ionization methods that are interfaced to LC are ESI and Atmospheric Chemical Ionization (APCI) in positive and negative-ion modes. The LC is done in most cases by RP-HPLC, and the buffer system should not contain involatile salts (e.g., phosphates). ESI can be used for m/z 500-4000 and is done at low resolving power. LC-MS can be used to look at a wide variety of biologically important compounds including, peptides, proteins, oligonucleotides, and lipids.

The chromatography for gene expression profiling or genotyping by LC/MS can be performed using a ProStar Helix System (catalog # Helixsys01) which is composed of two pumps, a column oven, a UV detector, a degasser, a mixer and an autoinjector. The column is like a Varian Microsorb MV (catalog number R0086203F5), C18 packing with 5 uM particle size, with 300 Angstroms pore size, 4.6 mm×50 mm. The column can be run at 30° C. to 40° C. with a gradient of acetonitrile in 100 mM Triethylamine acetate (TEAA) and 0.1 mM EDTA. The following HPLC method can be used to separation the fragments on the column: Buffer A is 100 mM TEAA with 0.1 mM EDTA, Buffer B is 100 mM TEAA with 0.1 mM EDTA and 25% (V/V) acetonitrile, 0-3 minutes there is a gradient of 20% B to 25% B, at 3.01 minutes to 4 minutes, there is a ramp to 45% B, at 4.01 to 4.5 minutes there is a ramp to 95% B, at 4.51 minutes there is 1 minutes hold at 20% B to re-equilibrate the column. The column can be run at 30-50 C. by adjusting the column oven to 30 C. to 50 C. The flow rate can be 0.5 to 1.5 ml per minute. About 1 to 200 nanogram of fragment can be injected per 10-50 microliter volume. The UV detector measures the effluent of the column.

High-Performance Liquid Chromatography (HPLC) is a chromatographic technique for separation of compounds dissolved in solution. HPLC instruments consist of a reservoir of mobile phase, a pump, an injector, a separation column, and a detector. Compounds are separated by injecting an aliquot of the sample mixture onto the column. The different components in the mixture pass through the column at different rates due to differences in their partitioning behavior between the mobile liquid phase and the stationary phase. The pumps provide a steady high performance with no pulsating, and can be programmed to vary the composition of the solvent during the course of the separation.

Exemplary detectors useful within the methods of present invention include UV-VIS absorption, or fluorescence after excitation with a suitable wavelength, mass spectrometers and IR spectrometers. Oligonucleotides labeled with fluorochromes may replace radio-labeled oligonucleotides in semi-automated sequence analysis, minisequencing and genotyping. (Smith et al., Nature 321:674 (1986)).

IP-RO-HPLC on non-porous PS/DVB particles with chemically bonded alkyl chains may be employed in the analysis of both single and double-strand nucleic acids. (Huber et al., Anal. Biochem. 212:351 (1993); Huber et al., Nuc. Acids Res. 21:1061 (1993); Huber et al., Biotechniques 16:898 (1993)). In contrast to ion-exchange chromatography, which does not always retain double-strand DNA as a function of strand length (since AT base pairs interact with the positively charged stationary phase, more strongly than GC base-pairs), IP-RP-HPLC enables a strictly size-dependent separation.

A method has been developed using 100 mM triethylammonium acetate as ion-pairing reagent, phosphodiester oligonucleotides could be successfully separated on alkylated non-porous 2.3 μM poly(styrene-divinylbenzene) particles by means of high performance liquid chromatography. (Oefner et al., Anal. Biochem. 223:39 (1994)). The technique described allows the separation of PCR products differing by only 4 to 8 base pairs in length within a size range of 50 to 200 nucleotides.

Denaturing HPLC (DHPLC) is an ion-pair reversed-phase high performance liquid chromatography methodology (IP-RP-HPLC) that uses a non-porous C-18 column as the stationary phase. The column is comprised of a polystyrene-divinylbenzene copolymer. The mobile phase is comprised of an ion-pairing agent of triethylammonium acetate (TEAA), which mediates binding of DNA to the stationary phase, and acetonitrile (ACN) as an organic agent to achieve subsequent separation of the DNA from the column. A linear gradient of acetonitrile allows separation DHPLC identifies mutations and polymorphisms based on detection of heteroduplex formation between mismatched nucleotides in double stranded PCR amplified DNA. Sequence variation creates a mixed population of heteroduplexes and homoduplexes during reannealling of wild type and mutant DNA of fragments based on size and/or presence of heteroduplexes (this is the traditional use of the DHPLC technology). When this mixed population is analyzed by HPLC under partially denaturing temperatures, the heteroduplexes elute from the column earlier than the homoduplexes because of their reduced melting temperature. Analysis can be performed on individual samples to determine heterozygosity, or on mixed samples to identify sequence variation between individuals.

In certain applications, it may be preferred to use the DHPLC column in a non-denaturing mode in order to separate identically sized DNA fragments which possess a different nucleotide composition. For example, the non-denaturing mode may be applicable where, for example, a 6-mer contains a C→T single nucleotide polymorphism (SNP) such as where the wild-type single strand DNA fragment has the nucleotide sequence 5′-AACCCC-3′ and where the mutant single strand DNA fragment has the nucleotide sequence 5′-AATCCC-3′. Fragments as short as 1-mers, 2-, 3-, 4-, 5-, 6-, 7-, 8-, to 16-mers show different mobilities (retention times) on the DHPLC instrument. Alternative to applications employing non-porous materials for performing the chromatography of the small nucleic acid fragments generated by IPRE cleavage, HPLC as both sizing and DHPLC applications work on a wide pore silica based material. Porous materials have the advantage of high sample capacity for semipreparative work. This is marketed by HP as Eclipse dsDNA columns.

### 7. Software for Analysis of Sequence Information Derived From Detection Methodologies

Detection methodologies employed in the methods of the present invention may optionally employ one or more computer algorithms for analyzing the derived sequence information. Algorithms of the present invention may be encompassed within software packages that convert a detection signal, such as a mass-to-charge ratio of a given small nucleic acid fragment, to a genotyping call.

Exemplary software packages may comprise the following: a peak identification algorithm which identifies peaks above a certain threshold of intensity (area under the curve), an algorithm that identifies and records the mass to charge ratio of the peaks between the scan intervals, an algorithm that calculates the intensity of peaks by measuring the area under the curve, an algorithm that calculates the number of peaks during a scan interval, an algorithm that calculates the ratio of each set of two peaks, an algorithm that calculates the allele calling from the ratiometric values.

The software package and algorithms may record the sample identification (sample ID), source, primer name and sequence, mass to charge ratio of expected fragment, estimation of expected mass to charge ratio, mass spectrometry details, sample plate ID, sample well ID, date and time, number of peaks observed, observed mass to charge ratio, and calculated allele call. The algorithms may also download the data to existed databases and check for accuracy of recording.

A complete genotyping system for use with a mass spectrometry detection system can be composed of the following: A computer (e.g., a Dell Optiplex Gx 110, with a CD-ROM), a software package to control the mass spectrometry, a thermocycler, and a robot that moves microtiter plates on and off the autoinjector, a simple HPLC to desalt the PCR or amplification reaction, the mass spectrometer such as an Agilent LC-quadrupole, ES-TOF, a Micromass ES-TOF or APCI-quadrupole, and a software program to call the alleles.

Alternatively, a complete genotyping system for use with a fluorescence based detection system can be composed of the following: a peak identification algorithm which identifies peaks above a certain threshold of intensity (area under the curve), an algorithm that identifies and records the retention time of the peaks between certain time intervals (ie., between 1.75 and 3 minutes in a 5 minute run), an algorithm that calculates the intensity of peaks by measuring the area under the curve, an algorithm that calculates the number of peaks between a certain time interval (i.e., between 1.75 and 3 minutes in a 5 minute run), an algorithm that calculates the ratio of each set of two peaks, an algorithm that calculates the allele calling from the ratiometric values. The software package and algorithms record the sample identification (sample ID), source, primer name and sequence, length of expected fragment, estimation of expected retention time, chromatography details, sample plate ID, sample well ID, date and time, number of peaks observed, observed retention times, and calculated allele call. The algorithms will also download the data to existed databases and check for accuracy of recording.

The software package that converts the presence of a label on the fragment to a genotyping call is composed of the following: an algorithm that calculates the allele calling from the ratiometric values of fluorescence or fluorescence polarization. The software package and algorithms record the sample identification (sample ID), source, primer name and sequence, mass to charge ratio of expected fragment, estimation of expected fluorescence ratios, instrument details, sample plate ID, sample well ID, date and time, and calculated allele call. The algorithms will also download the data to existing databases and check for accuracy of recording.

The software package that converts the mass to charge ratio of the fragment to a genotyping call is composed of the following: an algorithm that records the temporal parameter of the chromatography, a peak identification algorithm which identifies peaks above a certain threshold of intensity (area under the curve), an algorithm that identifies and records the mass to charge ratio of the peaks between the scan intervals, an algorithm that calculates the intensity of peaks by measuring the area under the curve, an algorithm that calculates the number of peaks during a scan interval, an algorithm that calculates the ratio of each set of two peaks, an algorithm that calculates the allele calling from the ratiometric values. The software package and algorithms record the sample identification (sample ID), source, primer name and sequence, mass to charge ratio of expected fragment, estimation of expected mass to charge ratio, chromatography details, elution time of each fragments, mass spectrometry details, sample plate ID, sample well ID, date and time, number of peaks observed, observed mass to charge ratio, and calculated allele, sequence identity, or gene identity call. The algorithms will also download the data to existed databases and check for accuracy of recording.

### C. Applications for the Methods, Compositions and Compounds of the Present Invention in the Detection of Mutations and Defined Nucleotide Loci

As discussed in detail herein above, the present invention provides methodology for the detection of mutations at defined nucleotide loci within target nucleic acids and/or measurement of genetic variations in parallel. Also provided herein, are various “readout” technologies that may be employed with the methodologies of the present invention for detecting, for example, the size and/or molecular weight of one or more single-strand fragment comprising the mutations and/or genetic variations. Methods according to the present invention will find utility in a wide variety applications wherein it is necessary to identify such a mutation at a defined nucleotide locus or measure genetic variations. Such applications include, but are not limited to, genetic analysis for hereditary diseases, tumor diagnosis, disease predisposition, forensics or paternity, crop cultivation and animal breeding, expression profiling of cell function and/or disease marker genes, and identification and/or characterization of infectious organisms that cause infectious diseases in plants or animals and/or that are related to food safety. Furthermore, the present methods may be utilized to greatly increase the specificity, sensitivity and throughput of the assay while lowering costs in comparison to conventional methods currently available in the art. Described below are certain exemplary applications of the present invention.

### 1. Expression Profiling

Most mRNAs are transcribed from single copy sequences. Another property of cDNAs is that they represent a longer region of the genome because of the introns present in the chromosomal version of most genes. The representation varies from one gene to another but can be very significant as many genes cover more than 100 kb in genomic DNA, represented in a single cDNA. One possible use of molecular profiling is the use of probes from one species to find clones made from another species. Sequence divergence between the mRNAs of mouse and man permits specific cross-reassociation of long sequences, but except for the most highly conserved regions, prevents cross-hybridization of PCR primers.

Differential screening in complex biological samples such as developing nervous system using cDNA probes prepared from single cells is now possible due to the development of PCR-based and cRNA-based amplification techniques. Several groups reported previously the generation of cDNA libraries from small amounts of poly (A)+RNA (1 ng or less) prepared from 10-50 cells (Belyav et al., Nuc. Acids Res. 17:2919, 1989). Although the libraries were sufficiently representative of mRNA complexity, the average cDNA insert size of these libraries was quite small (<2 kb).

More recently, methodologies have been combined to generate both PCR-based (Lambolez et al., Neuron 9:247, 1992) and cRNA-based (Van Gelder et al., Proc. Natl. Acad. Sci. USA 87:1663, 1990) probes from single cells. After electrical recordings, the cytoplasmic contents of a single cell were aspirated with patch-clamp microelectrodes for in situ cDNA synthesis and amplification. PCR was used to amplify cDNA of selective glutamate receptor mRNAs from single Purkinje cells and GFAP mRNA from single glia in organotypic cerebellar culture (Lambolez et al., Neuron 9:247, 1992). In the case of cRNA amplification, transcription promoter sequences were designed into primers for cDNA synthesis and complex antisense cRNAs were generated by in vitro transcription with bacteriophage RNA polymerases.

The array of the present invention is useful for determining whether a particular cDNA molecule is present in cDNAs from a biological sample and further determine whether genetic variation(s) exist in the cDNA molecule.

### 2. Forensics

The identification of individuals at the level of DNA sequence variation offers a number of practical advantages over such conventional criteria as fingerprints, blood type, or physical characteristics. In contrast to most phenotypic markers, DNA analysis readily permits the deduction of relatedness between individuals such as is required in paternity testing. Genetic analysis has proven highly useful in bone marrow transplantation, where it is necessary to distinguish between closely related donor and recipient cells. Two types of probes are now in use for DNA fingerprinting by DNA blots. Polymorphic minisatellite DNA probes identify multiple DNA sequences, each present in variable forms in different individuals, thus generating patterns that are complex and highly variable between individuals. VNTR probes identify single sequences in the genome, but these sequences may be present in up to 30 different forms in the human population as distinguished by the size of the identified fragments. The probability that unrelated individuals will have identical hybridization patterns for multiple VNTR or minisatellite probes is very low. Much less tissue than that required for DNA blots, even single hairs, provides sufficient DNA for a PCR-based analysis of genetic markers. Also, partially degraded tissue may be used for analysis since only small DNA fragments are needed. The methods of the present invention are useful in characterizing polymorphism of sample DNAs, therefore useful in forensic DNA analyses. For example, the analysis of 22 separate gene sequences in a sample, each one present in two different forms in the population, could generate 1010 different outcomes, permitting the unique identification of human individuals.

### 3. Tumor Diagnostics

The detection of viral or cellular oncogenes is another important field of application of nucleic acid diagnostics. Viral oncogenes (v-oncogenes) are transmitted by retroviruses while their cellular counterparts (c-oncogenes) are already present in normal cells. The cellular oncogenes can, however, be activated by specific modifications such as point mutations (as in the c-K-ras oncogene in bladder carcinoma and in colorectal tumors), small deletions and small insertions. Each of the activation processes leads, in conjunction with additional degenerative processes, to an increased and uncontrolled cell growth. In addition, point mutations, small deletions or insertions may also inactivate the so-called “recessive oncogenes” and thereby leads to the formation of a tumor (as in the retinoblastoma (Rb) gene and the osteosarcoma). Accordingly, the present invention is useful in detecting or identifying the point mutations, small deletions and small mutations that activate oncogenes or inactivate recessive oncogenes, which in turn, cause cancers.

### 4. Transplantation Analyses

The rejection reaction of transplanted tissue is decisively controlled by a specific class of histocompatibility antigens (HLA). They are expressed on the surface of antigen-presenting blood cells, e.g., macrophages. The complex between the HLA and the foreign antigen is recognized by T-helper cells through corresponding T-cell receptors on the cell surface. The interaction between HLA, antigen and T-cell receptor triggers a complex defense reaction which leads to a cascade-like immune response on the body.

The recognition of different foreign antigens is mediated by variable, antigen-specific regions of the T-cell receptor-analogous to the antibody reaction. In a graft rejection, the T-cells expressing a specific T-cell receptor which fits to the foreign antigen, could therefore be eliminated from the T-cell pool. Such analyses are possible by the identification of antigen-specific variable DNA sequences which are amplified by PCR and hence selectively increased. The specific amplification reaction permits the single cell-specific identification of a specific T-cell receptor.

Similar analyses are presently performed for the identification of auto-immune disease like juvenile diabetes, arteriosclerosis, multiple sclerosis, rheumatoid arthritis, or encephalomyelitis.

Accordingly, the present invention is useful for determining gene variations in T-cell receptor genes encoding variable, antigen-specific regions that are involved in the recognition of various foreign antigens.

### 5. Genome Diagnostics

Four percent of all newborns are born with genetic defects; of the 3,500 hereditary diseases described which are caused by the modification of only a single gene, the primary molecular defects are only known for about 400 of them.

Hereditary diseases have long since been diagnosed by phenotypic analyses (anamneses, e.g., deficiency of blood: thalassemias), chromosome analyses (karyotype, e.g., mongolism: trisomy 21) or gene product analyses (modified proteins, e.g., phenylketonuria: deficiency of the phenylalanine hydroxylase enzyme resulting in enhanced levels of phenylpyruvic acid). The additional use of nucleic acid detection methods considerably increases the range of genome diagnostics.

In the case of certain genetic diseases, the modification of just one of the two alleles is sufficient for disease (dominantly transmitted monogenic defects); in many cases, both alleles must be modified (recessively transmitted monogenic defects). In a third type of genetic defect, the outbreak of the disease is not only determined by the gene modification but also by factors such as eating habits (in the case of diabetes or arteriosclerosis) or the lifestyle (in the case of cancer). Very frequently, these diseases occur in advanced age. Diseases such as schizophrenia, manic depression or epilepsy should also be mentioned in this context; it is under investigation if the outbreak of the disease in these cases is dependent upon environmental factors as well as on the modification of several genes in different chromosome locations.

Using direct and indirect DNA analysis, the diagnosis of a series of genetic diseases has become possible: bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease. The present application is useful in diagnosis of any genetic diseases that are caused by point mutations, small deletions or small insertions at defined positions.

### 6. Infectious Disease

The application of recombinant DNA methods for diagnosis of infectious diseases has been most extensively explored for viral infections where current methods are cumbersome and results are delayed. In situ hybridization of tissues or cultured cells has made diagnosis of acute and chronic herpes infection possible. Fresh and fomalin-fixed tissues have been reported to be suitable for detection of papillomavirus in invasive cervical carcinoma and in the detection of HIV, while cultured cells have been used for the detection of cytomegalovirus and Epstein-Barr virus. The application of recombinant DNA methods to the diagnosis of microbial diseases has the potential to replace current microbial growth methods if cost-effectiveness, speed, and precision requirements can be met. Clinical situations where recombinant DNA procedures have begun to be applied include the identification of penicillin-resistant Neisseria gonorrhoeae by the presence of a transposon, the fastidiously growing chlamydia, microbes in foods; and simple means of following the spread of an infection through a population. The worldwide epidemiological challenge of diseases involving such parasites as leishmania and plasmodia is already being met by recombinant methods.

The present invention is useful to detect and/or measure genetic variations that are involved in infectious diseases, especially those in drug resistance genes. Thus, the present invention facilitates the characterization and classification of organisms that cause infectious diseases and consequently the treatment of such diseases caused by these organisms.

### EXAMPLES

The following experimental examples are offered by way of illustration, not limitation.

### Example 1

### DETECTION OF OLIGONUCLEOTIDE FRAGMENTS WITH ELECTROSPRAY-LIQUID CHROMATOGRAPHY/MASS SPECTROMETRY

This example discloses the use of Electrospray-Liquid-Chromatography/Mass Spectrometry (ES-LC/MS) for determining the molecular weight of single-strand oligonucleotide (ODN) fragments of 4, 6, 8 and 10 nucleotides in length (Table 4).

ODNs were synthesized by Midland Certified reagents of Midland Tex. The ODNs were diluted to a concentration of 0.5 nm/ul in 0.01 M Tris-HCl, 0.1 mM EDTA to create a stock solution. Each stock solution of ODN was subsequently diluted 1:10, 1:100 and 1:1000 in purified water. Five microliters of each dilution was injected into a electrospray-liquid-chromatography/mass spectrometry, time-of flight (ES-LC/MS-TOF) system using negative ion full scan, cone=35 volts, source at 100° C., desolvation at 250° C., with a Xterra column, C8, 2.1×50 mm, with a flow rate of 300 μl/min., direct, running isocratic in water, methanol +0.05% TEA.

The chromatography was performed using the following system: a ProStar Helix System (catalog # Helixsys01) which is composed of two pumps, a column oven, a UV detector, a degasser, a mixer and an autoinjector. The column is a Varian Microsorb MV (catalog number R0086203F5), C18 packing with 5 uM particle size, with 300 Angstroms pore size, 4.6 mm×50 mm. The column was run at 30° C. to 40° C. with a gradient of acetonitrile in 100 mM Triethylamine acetate (TEAA) and 0.1 mM EDTA. The following HPLC method was used to separate the fragments on the column: Buffer A is 100 mM TEAA with 0.1 mM EDTA, Buffer B is 100 mM TEAA with 0.1 mM EDTA and 25% (V/V) acetonitrile, 0-3 minutes there is a gradient of 20% B to 25% B, at 3.01 minutes to 4 minutes, there is a ramp to 45% B, at 4.01 to 4.5 minutes there is a ramp to 95% B, at 4.51 minutes there is 1 minutes hold at 20% B to re-equilibrate the column. The column was run at 40° C. by adjusting the column oven to 40° C. The flow rate was 1.5 ml per minute. About 200 nanogram of fragment was injected per 10 microliter volume. The UV detector measures the effluent of the column.

TABLE 4

| | | | MOLECULAR | |
|---|---|---|---|---|
| | | | WEIGHT | |
| ODN | SEQUENCE | LENGTH | (Daltons) | SEQ ID NO. |
| Fok0001 | 5′-ACGA-3′ | 4-mer | 1181 Da | |
| Fok0002 | 5′-ACGATG-3′ | 6-mer | 1816 Da | |
| Fok0003 | 5′-ACGATGCA-3′ | 8-mer | 2418.6 Da | |
| Fok0004 | 5′-GAACATCCAT-3′ | 10-mer | 2996 Da | SEQ ID NO: 8 |

The molecular weights of the ODNs detailed in Table 4 were measured by ES-LC/MS and the lower limit of detection was determined.

For the 4-mer with an expected molecular weight of 1181 Daltons, the total extracted ion current between 1181.102 and 1181.7 was 0.41. FIG. 1 shows the chromatogram of 1/10 dilution of the 4-mer with the 1181.4 peak normalized to 100%. The 1181.4 peak represents the singly charged species in which the mass/charge (m/z) ratio=1. A doubly charged 4-mer with a mass of 590.2 appears at 80% the intensity of the 1181.4 species. The 590.2 mass represents an m/z value of 0.5 that of the singly charged species (i.e., the 590.2 species has two charges). Also note that an n+0.5 and n+1 charge was seen at 590.7 and 591.2. This is typical with electrospray ionization with polymeric molecules.

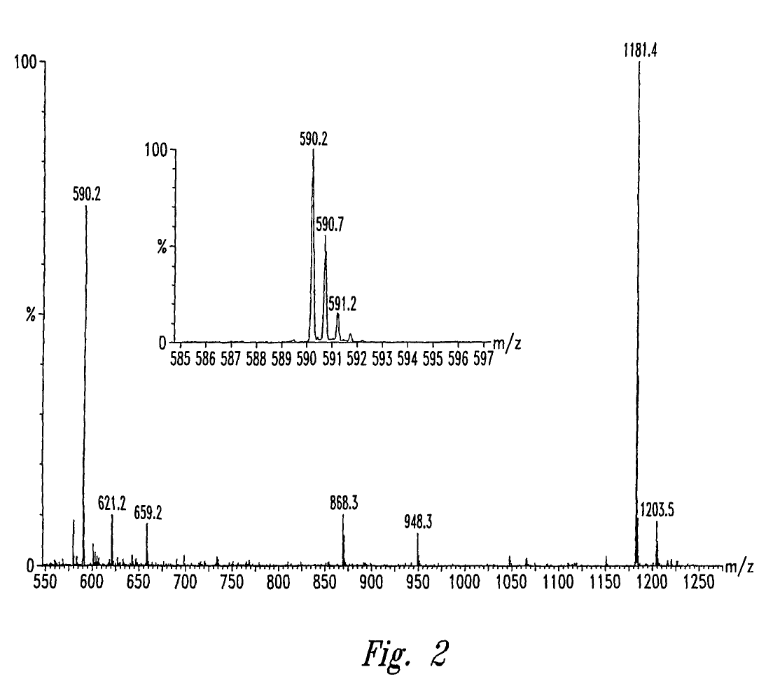

At a 1/100 dilution, the mass spectra looks similar to the 1/10 dilution, with peaks of expected molecular weight 1181.4 and 590.2. The peak at 1203.5 is the Na+ adduct (+23 Daltons) of the 4-mer. The peaks at 621.2, 659.2, 868.3, 948.3 are either fragmentation products or background (FIG. 2).

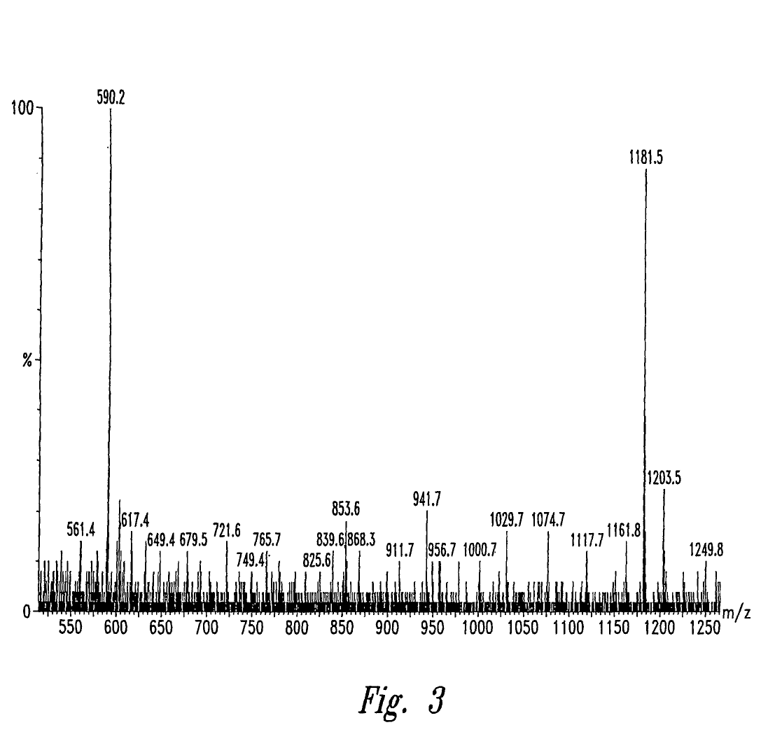

In FIG. 3, the dilution at 1/1000 is shown which is about at the lower limit of detection for the ES-TOF. Peaks at 1181.5 and 590.2 are clearly visible above the background. More “background” is visible in the spectra as the lower limits of detection are being pushed.

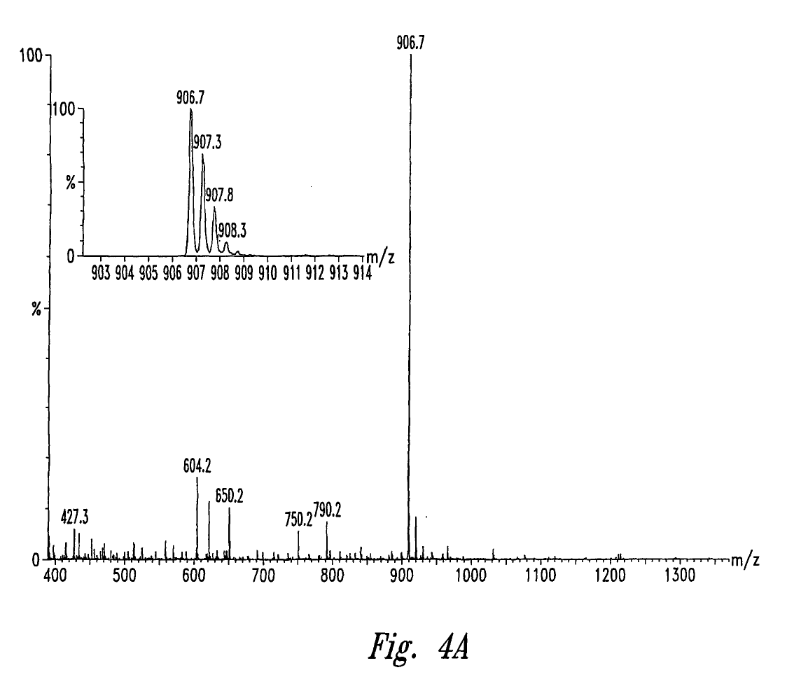

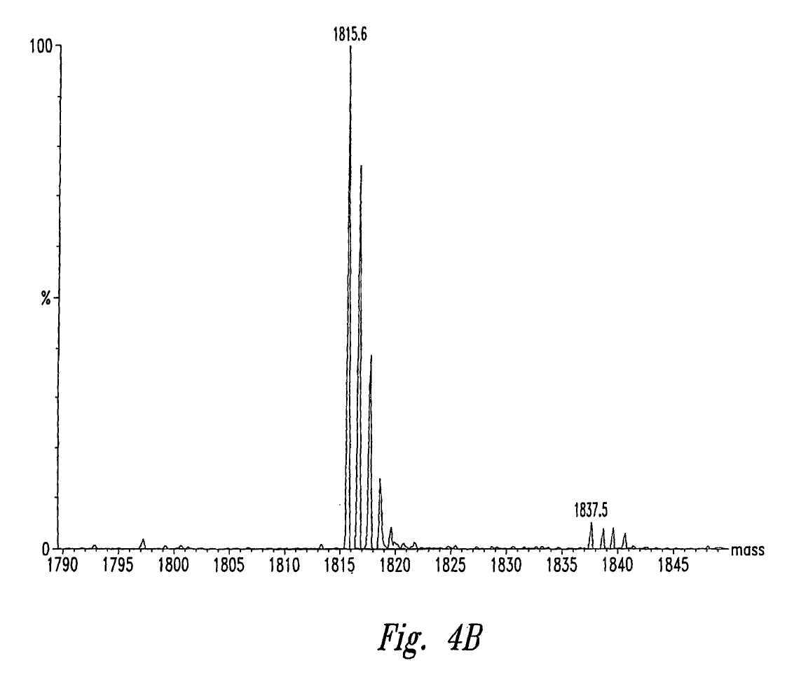

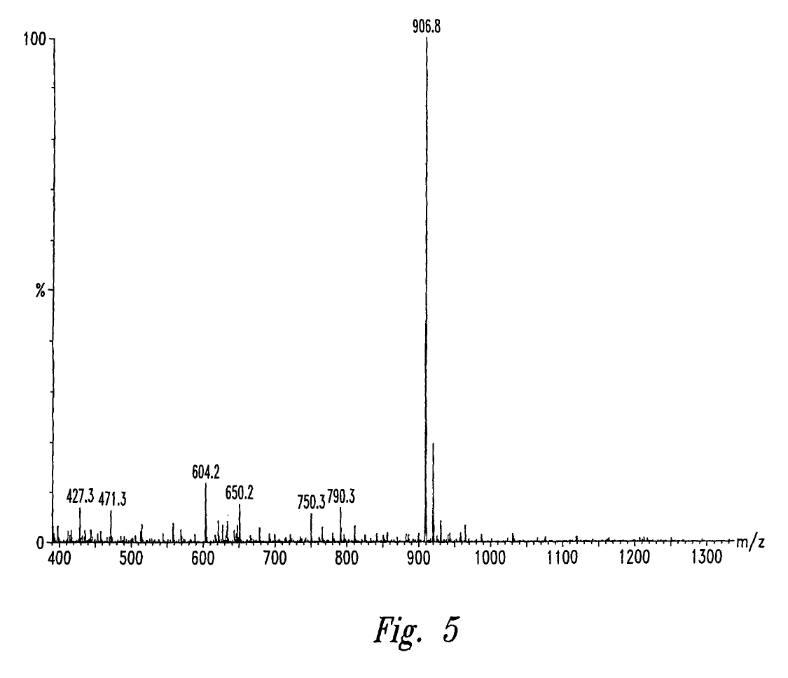

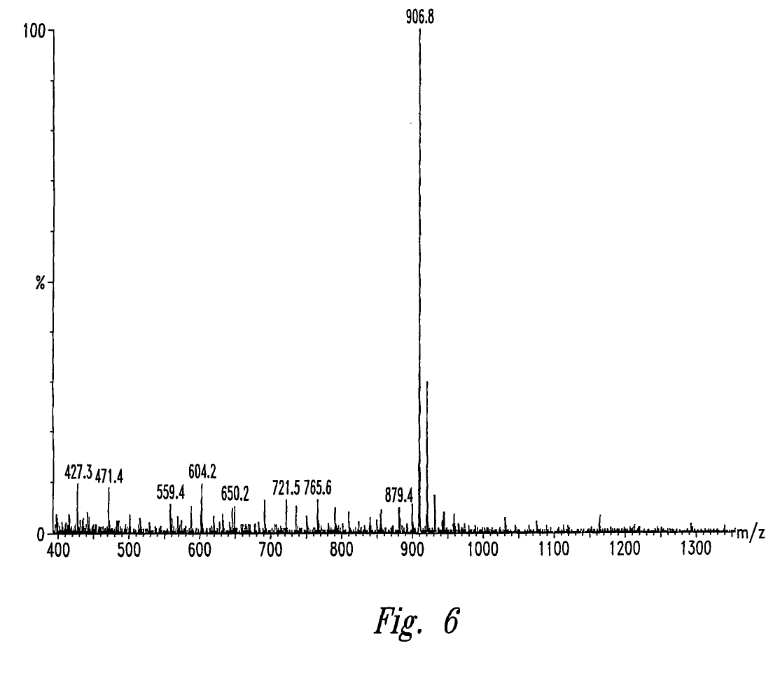

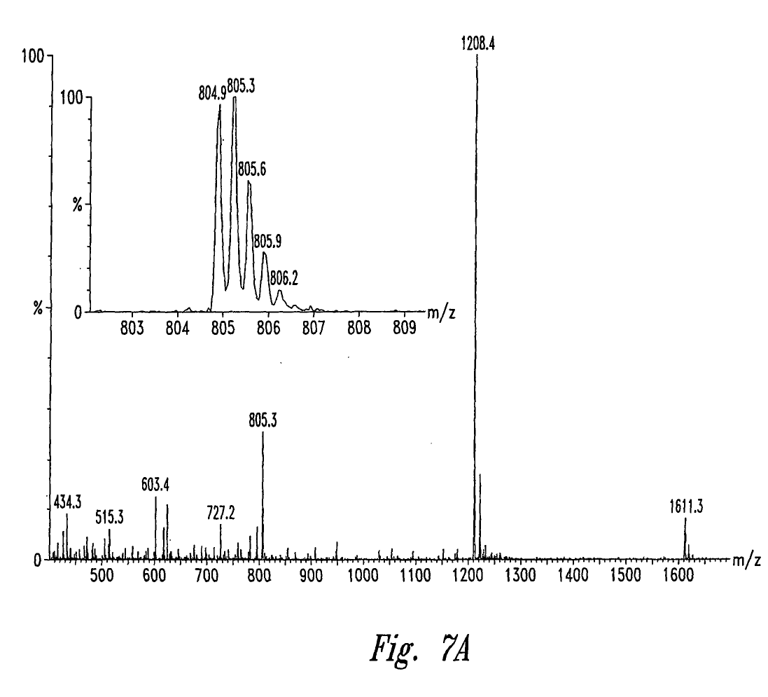

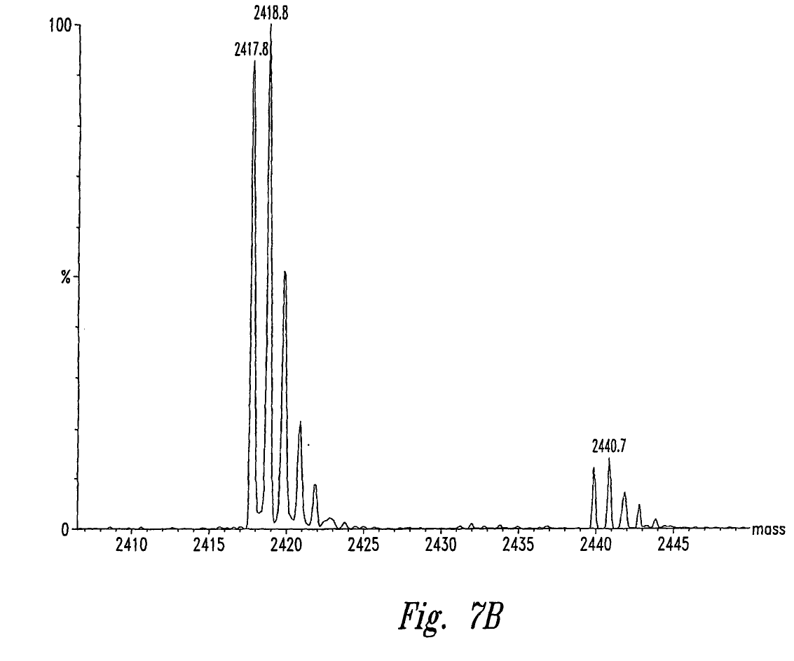

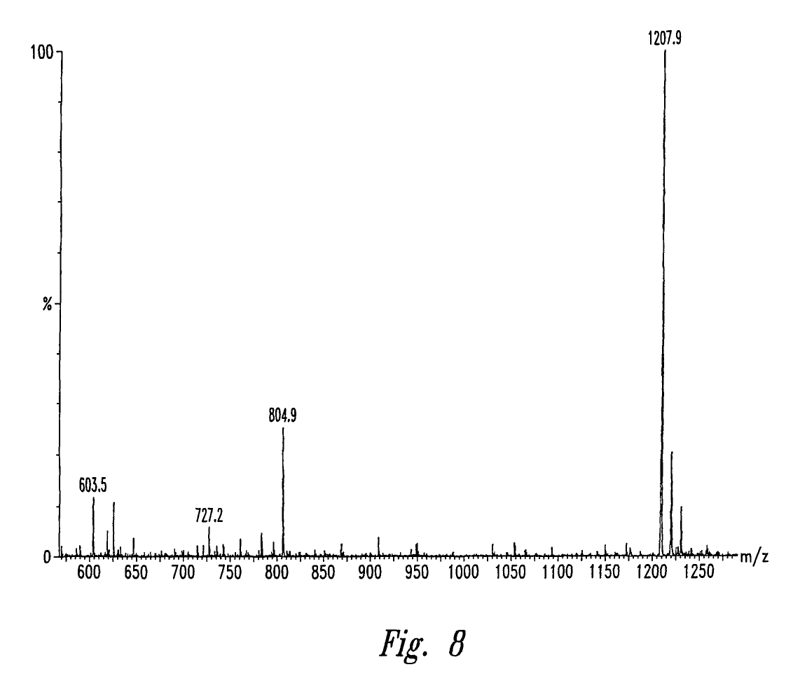

For the 6-mer with an expected molecular weight of 1816 Daltons, the total extracted ion current between 906.6 and 907.5 was 0.35 (the doubly charged species). FIG. 4A shows the chromatogram of 1/10 dilution of the 6-mer with the 906.7 peak normalized to 100% (this is the doubly charged species where m/z=0.5 (mass/charge=0.5). The 906.7 mass represents an m/z value of 0.5 that of the singly charged species (i.e., the 906.7 species has two charges.). Also note that an n+0.5 and n+1 charge is seen at 907.3 and 907.8. This is typical with electrospray ionization with polymeric molecules. In FIG. 4B, the mass spectrum shows peaks of expected molecular weight 1815.6. The peak at 1837.5 is the Na+ adduct (+23 Daltons) of the 6-mer. FIG. 5 shows the chromatogram of the 1/100 dilution of the 6 mer with the 906.8 peak normalized to 100%. In FIG. 6, the dilution at 1/1000 is shown which is about at the lower limit of detection for the ES-TOF. The peak at 906.8 is clearly visible above the background. More “background” is visible in the spectra as the lower limits of detection are being pushed.

For the 8-mer with an expected molecular weight of 2418.6 Daltons, the total extracted ion current between 1207.602 and 1210.1 was 0.37 (the doubly charged species). FIG. 7, panels A and B, show the chromatogram of 1/10 dilution of the 8-mer with the 1208.4 peak normalized to 100% (this is the doubly charged species where m/z=0.5 (mass/charge=0.5). The 805.3 mass represents an m/z value of 0.33 that of the singly charged species (i.e., the 805.3 species has three charges.). Also note that an n+0.5 and n+1 charge is seen at 805.9 and 806.3. This is typical with electrospray ionization with polymeric molecules. At a 1/100 dilution (FIG. 8), the mass spectra shows peaks of expected molecular weight 1207.9 (doubly charged) and 804.9 (triply charged).

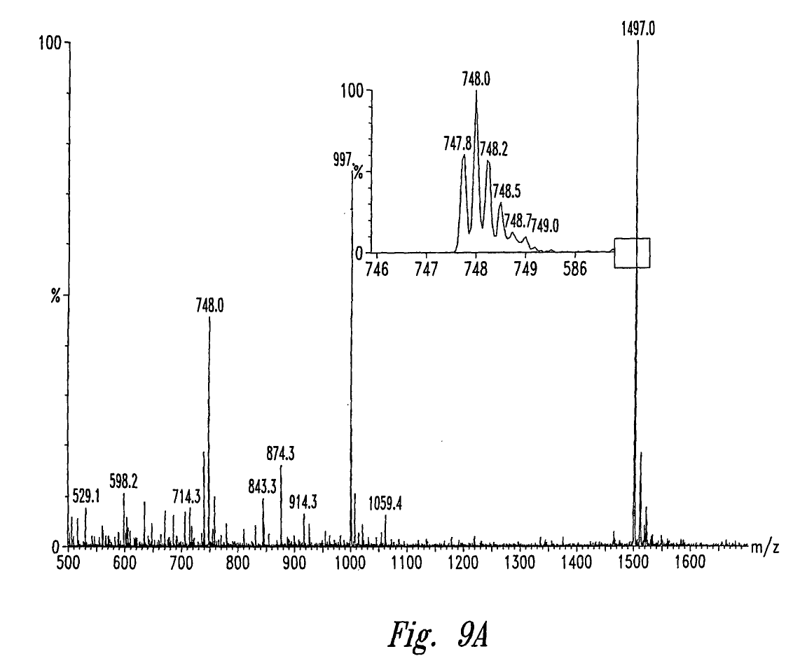

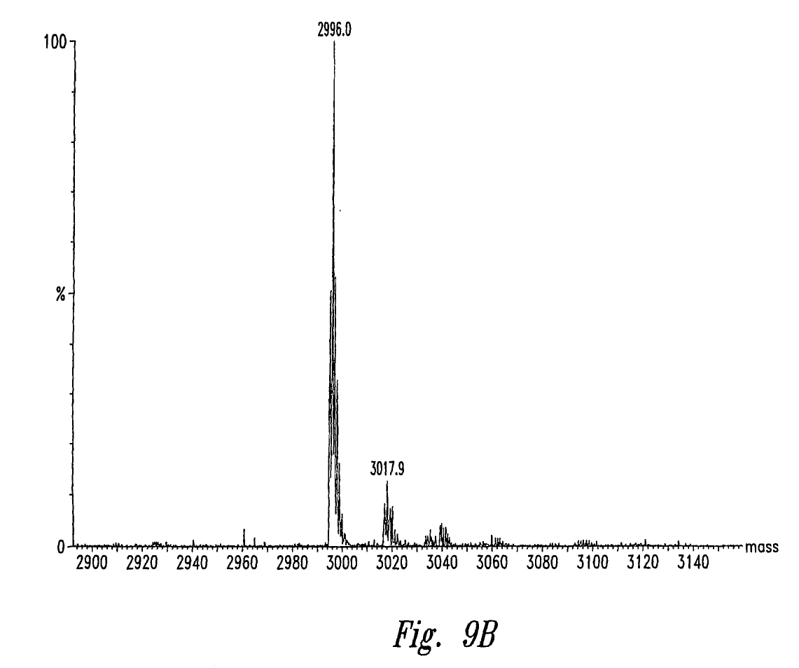

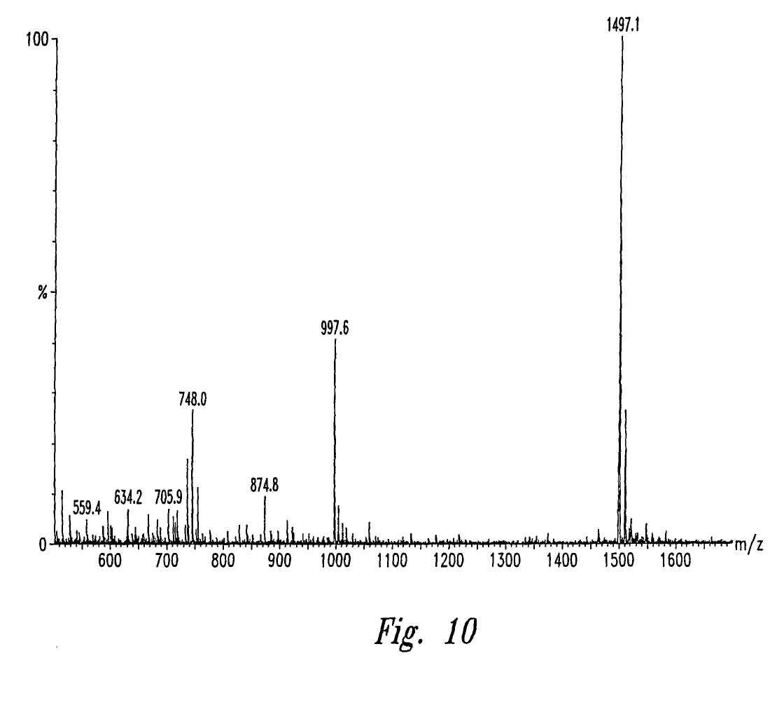

For the 10-mer with an expected molecular weight of 2996 Daltons, the total extracted ion current between 1496.199 and 1500.182 was 0.37 (the doubly charged species). FIGS. 9A and B shows the chromatogram of 1/10 dilution of the 10-mer with the 1497.0 peak normalized to 100% (this is the doubly charged species where m/z=0.5 (mass/charge=0.5). The 1497.0 mass represents an m/z value of 0.5 that of the singly charged species (i.e., the 1497 species has two charges.). Also note that an n+0.5 and n+1 charge is seen at 748.5 and 749 for the species with 4 charges. This is typical with electrospray ionization with polymeric molecules. FIG. 9B shows the mass spectra with peaks of expected molecular weight 2996. The peak at 3017.9 is the Na+ adduct (+23 Daltons) of the 10-mer. FIG. 10 shows the 1/100 dilution of the 10-mer with the 1497.1 peak normalized to 100%. In FIG. 11. the dilution at 1/1000 is

### Example 2

### NON-PALINDROMIC RESTRICTION ENDONUCLEASE BASED METHODOLOGY FOR DETECTING A SINGLE NUCLEOTIDE POLYMORPHISM AT A DEFINED LOCATION IN A TARGET NUCLEIC ACID

This example discloses a methodology for determining the presence of a single nucleotide polymorphism (SNP) at a defined location within a target nucleic acid by measuring the molecular weight of a single-strand nucleic acid fragment released after digestion of an amplicon with a restriction endonuclease.

In this example, two SNPs are discriminated using the generation of a small fragment by Fok I digestion which is then measured using mass spectrometry (ES-TOF). The forward and reverse primers each contain a Fok I recognition sequence. The primers are used to amplify an allele of interest from the lambda genome. The Fok I cutting sites are designed such that after the restriction digest is performed, a fragment of 6 or 10 nucleotides is formed, depending on the primer set used.

An amplicon was generated by PCR amplifying the genomic DNA of SEQ ID NO: 13 (Table 6) using the first ODNP and second ODNP of primer set 1 (Table 5, i.e., SEQ ID NOs: 9 and 10, respectively). This amplicon was digested with Fok I to release a 10-mer single-stranded nucleic acid fragment comprising the sequence 5′-ATTATTCAGC-3′ (SEQ ID NO: 15).

An amplicon was generated by PCR amplifying the genomic DNA of SEQ ID NO: 14 (Table 6) using the first ODNP and second ODNP of primer set 2 (Table 5, i.e., SEQ ID NOs: 11 and 12, respectively). This amplicon was digested with Fok I to release a 6-mer single-stranded nucleic acid fragment comprising the sequence 5′-TTATTA-3′.

TABLE 5
Primer Sets for Generating Amplicons from Target Nucleic Acid

| | NAME OF FIRST | | NAME OF | |
|---|---|---|---|---|
| PRIMER | ODNP | SEQUENCE OF | SECONDODNP | SEQUENCE OF |
| SET | (SEQ ID NO) | FIRST ODNP | (SEQ ID NO) | SECOND ODNP |
| 1 | RE5P01 | 5′- | RE5P02 | 5′- |
| | (SEQ ID NO: 9) | GAAGTGATGGGGA | (SEQ ID NO: | GTAAGCCACACATC |
| | | TGCGGAAAGAG-3′ | 10) | CAGGAACGGG-3′ |
| 2 | RE5P03 | 5′- | RE5P04 | 5′- |
| | (SEQ ID NO: 11) | AAAGCTGGCAGGA | (SEQ ID NO: | AGCGTCTGTTCATC |
| | | TGACCGGCAGA-3′ | 12) | CTCGTGGCGG-3′ |

TABLE 6
Genomic Sequence Corresponding to each Primer Set

| PRI- | |
|---|---|
| MER | |
| SET | GENOMIC SEQUENCE (SEQ ID NO) |
| 1 | 5′-gaagtgatggcagagcggaaagagcattattcagcgcccgttcctgaccgtgtggcttac- |
| | 3′ (SEQ ID NO: 13) |
| 2 | 5′-gaaagctggctgattgaccggcagattattatgggccgccacgacgatgaacagacgctg- |
| | 3′ (SEQ ID NO: 14) |

The 100 μl PCR reactions comprised 100 ng genomic DNA; 0.5 μM of each first ODNP and second ODNP; 10 mM Tris, pH 8.3; 50 mM KCl; 1.5 mM MgCl₂; 200 μM each dNTP; 4 units Taq™ DNA Polymerase (Boehringer Mannheim; Indianapolis, Ind.), and 880 ng TaqStart™ Antibody (Clontech, Palo Alto, Calif.). Thermocycling conditions were as follows: 94° C. for 5 minutes initial denaturation; 45 cycles of 94° C. for 30 seconds, 60° C. for 30 seconds, 72° C. for 1 minute; final extension at 72° C. for 5 minutes. An MJ Research 9600 thermocycler (MJ Research, Watertown, Mass.) was used for all PCR reactions. Products were visualized via a 2.0% agarose gel stained with ethidium bromide.

Five μl of each dilution was injected into a mass spectrometry system composed of: ES-LC/MS (electrospray-liquid-chromatography/mass spectrometry, time-of flight (TOF)), using negative ion full scan, cone=35 volts, source at 100° C., desolvation at 250° C., with a Xterra column, C8, 2.1×50 mm, with a flow rate of 300 microliters per minute, direct, running isocratic in water, methanol +0.05% TEA.

The results indicated that the mass of the wild-type (3002 Daltons for the 10-mer) was observed for the primer set 1 after Fok I cutting, and the wild-type mass (1782 for the 6-mer) was observed for the primer set 2 after Fok I cutting.

### Example 3

### SEPARATION AND IDENTIFICATION OF OLIGONUCLEOTIDE FRAGMENTS THAT DIFFER BY A SINGLE NUCLEOTIDE USING HPLC

This example describes the separation and identification of short genotyping DNA fragments by liquid chromatography. The identification of the DNA fragments is by UV absorbance and retention time on the column.

The chromatography system is from Varian (Walnut Creek, Calif.) and is a ProStar Helix System (catalog # Helixsys01) which is composed of two pumps, a column oven, a UV detector, a degasser, a mixer and an autoinjector. The column is a Varian Microsorb MV (catalog number R0086203F5), C18 packing with 5 uM particle size, with 300 Angstroms pore size, 4.6 mm×50 mm. The column was run at 30° C. to 40° C. with a gradient of acetonitrile in 100 mM Triethylamine acetate (TEAA) and 0.1 mM EDTA. The type of gradient is described in the text.

The following genotyping fragments, each containing a specific Single Nucleotide Polymorphism were tested and successfully separated.

4-merA: 5′-ACGA-3′

6-merA: 5′-ACGATG-3′

8-merA: 5′-ACGACGCA-3′

8-merB: 5′-ATGACGCA-3′

8-merC: 5′-ACGATGCA-3′

10-merA: 5′-GAATATCCAT-3′ (SEQ ID NO. 16)

10-merB: 5′-GAATATCCAC-3′ (SEQ ID NO. 17)

10-merC: 5′-GAACATCCAT -3′ (SEQ ID NO. 8)

The polymorphisms in the 8-mers and 10-mers are underlined. The 8-mers B and C differ from 8-mer A by only a single base. The 10-mers B and C differ from 10-mer A by only a single base.

The following HPLC method was used to separate the fragments on the column: Buffer A is 100 mM TEAA with 0.1 mM EDTA, Buffer B is 100 mM TEAA with 0.1 mM EDTA and 25% (V/V) acetonitrile, 0-3 minutes there is a gradient of 20% B to 25% B, at 3.01 minutes to 4 minutes, there is a ramp to 45% B, at 4.01 to 4.5 minutes there is a ramp to 95% B, at 4.51 minutes there is 1 minutes hold at 20% B to re-equilibrate the column. The column was run at 40° C. by adjusting the column oven to 40° C. The flow rate was 1.5 ml per minute. The injection volume was 10 microliters and 200 nanogram of fragment was injected per 10 microliter volume. Different combinations of the 4-mer, 6-mer, 8-mer and 10-mer were injected to determine the chromagraphic behavior.

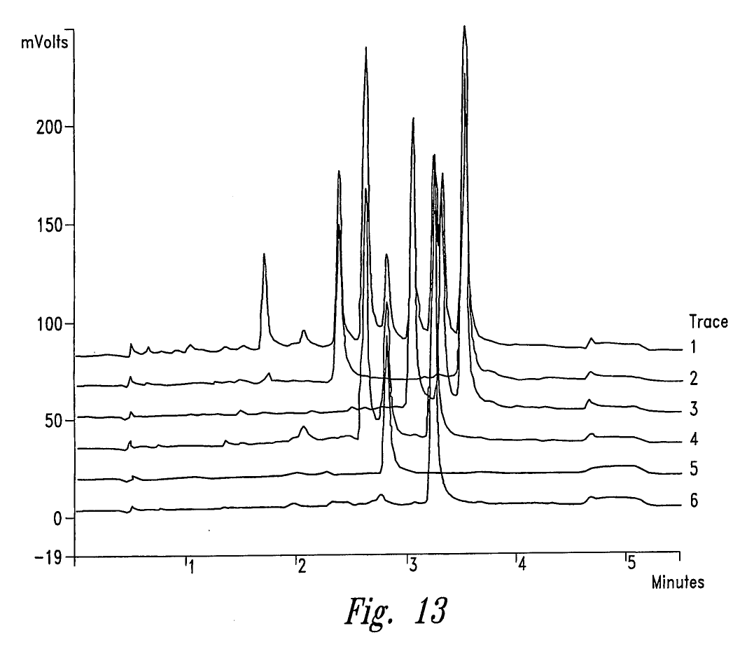

The first result is shown in FIG. 13. In Trace 1 in FIG. 13, all 8 fragments composed of the 4-mer, 6-mer, 8-mer and 10-mer were separated. All three 8-mers and all three 10-mers were separated even though they differed by only a single base. The fragments are single stranded. The order of elution in Trace 1 is (from left to right): 4-mer, 6-mer, 8-merB, 8-merA, 10-merA, 8-merC, 10-merB, 10merC. In Trace 2, the 6-mer and 10-merC were coinjected and the elution times of the 6-mer and 10-merC were the same as seen in Trace 1. In Trace 3, the 3 10-mers were co-injected and separated. The elution times of the 3 10-mers were the same as seen in Trace 1. In Trace 4, the 3 8-mers were co-injected and separated. The elution times of the 3 8-mers were the same as seen in Trace 1. Trace 5 shows a single peak of 8-merA and Trace 6 shows a single Trace of 8-merB. Genotypes can be directly inferred from the retention times during the chromatography, even from fragments that differ by only a single base.

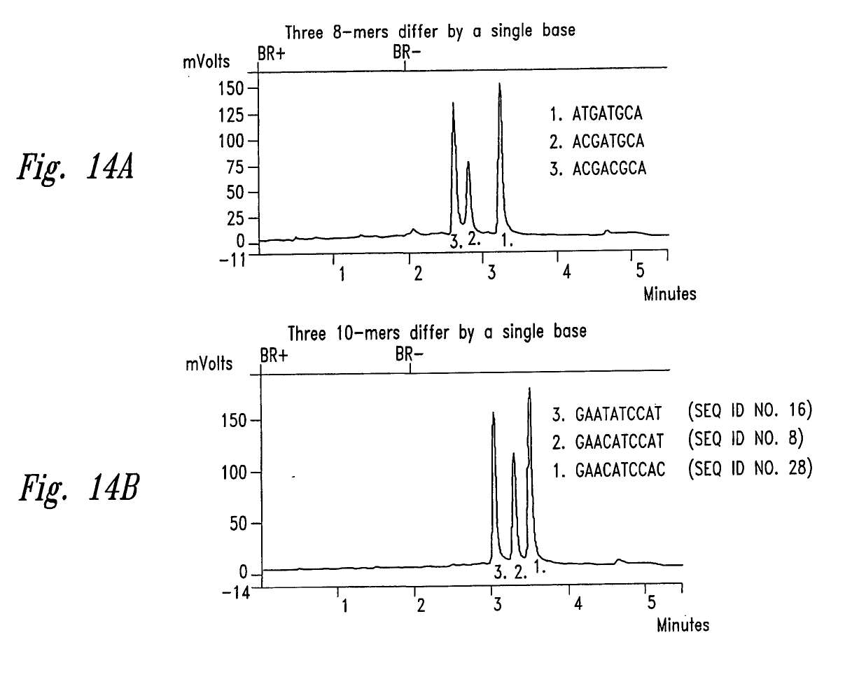

FIG. 14 shows an 8-mer genotyping result and a 10-mer genotyping result. In panel A, the “T” allele is position 2 is discriminated from the “C” allele in position 2 and the “C” allele in position 5 from the “T” allele in position 5. In panel B, the “T” allele is position 4 is discriminated from the “C” allele in position 4 and the “C” allele in position 10 from the “T” allele in position 10. Genotypes can be directly inferred from the retention times during the chromatography, even from fragments that differ by only a single base.

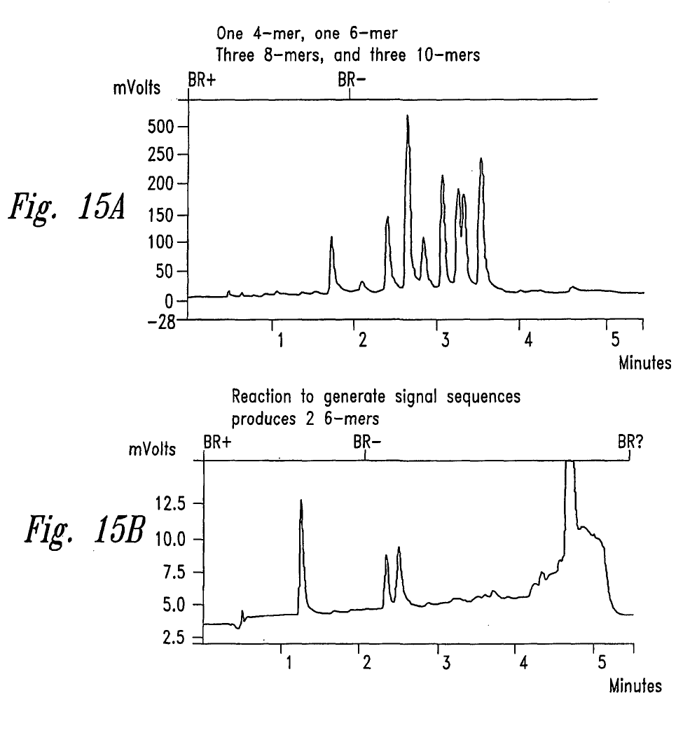

In FIG. 15, in Panel A, one 4-mer, one 6-mer, three 8-mers and three 10-mers are separated, and in panel B, two 6-mers are shown eluting between 2 and 3 minutes. The 6-mers were generated by double Fok I digestion of a 41-mer which contained the forward and reverse Fok I recognition site which was separated by 6 nucleotides.

### Example 4

### AMPLIFICATION, CUTTING AND DETECTION OF A GENOTYPING FRAGMENT USING THE FOK I NON-PALINDROMIC RESTRICTION ENDONCLEASE

The following example describes the amplification of a specific sequence from the lambda genome in which the primers contain the Fok I recognition sequence (both the forward and reverse primers). The resulting amplicon contains a “double-Fok I” cutting site which liberates a small oligonucleotide fragment, which is then subjected to a chromatography step and identified by UV absorbance.

Two sets of primers were designed to generate two different amplicons from two different regions of the lambda genome.

| | |
|---|---|
| RE5P01F: | |
| 5′-GAAGTGATGGGGATGCGGAAAGAG-3′ | (SEQ ID NO.9) |
| RE5P02R: | |
| 5′-GTAAGCCACAGGATGAGGAACGGG-3′ | (SEQ ID NO.18) |
| RE5P03F: | |
| 5′-AAAGCTGGCAGGATGACCGGCAGA-3′ | (SEQ ID NO.11) |
| RE5P04R: | |
| 5′-AGCGTCTGTTGGATGTCGTGGCGG-3′ | (SEQ ID NO.19) |

Where RE5P01F and RE5P02R are primer set one and RE5P03F and RE5P04R are primer set 2. All oligonucleotides were synthesized by Midland Reagent CO. of Midland Tex.

The following PCR reaction mixture was used in 25 μl volumes: The 25 μl PCR reactions were composed of 25 ng genomic DNA, 0.5 μM forward and reverse primers, 10 mM Tris pH 8.3, 50 mM KCl, 1.5 mM MgCl₂, 200 μM each dNTP, 4 Units Taq DNA Polymerase (Boehringer Mannheim, Indianapolis, Ind.), and 880 ng TaqStart Antibody (Clontech, Palo Alto, Calif.). Thermocycling conditions were as follows: 94° C. for 5 minutes initial denaturation; 45 cycles of 94° C. for 30 seconds, 60° C. for 30 seconds, 72° C. for 1 minute; fmal extension at 72° C. for 5 minutes. A MJ Research 9600 thermocycler (MJ Research, Watertown, Mass.) was used for all PCR reactions.

After the thermocycling was complete, 5 microliters of NEB buffer-4 (New England Biolabs, Beverly Mass.) and 5 microliters of Fok I enzyme (New England Biolabs, Beverly Mass.), 20 units, were added, giving final concentrations of 50 mM potassium acetate, 20 mM Tris acetate, 10 mM Mg acetate, 1 mM DTT, pH 7.9. The reaction was carried out at 37° C. for 30 minutes. The reaction was injected directly without any further purification.

The following HLC method was used to separate the fragments on the column: Buffer A is 100 mM TEAA with 0.1 mM EDTA, Buffer B is 100 mM TEAA with 0.1 mM EDTA and 25% (VIV) acetonitrile, 0-3 minutes there is a gradient of 20% B to 25% B, at 3.01 minutes to 4 minutes, there is a ramp to 45% B, at 4.01 to 4.5 minutes there is a ramp to 95% B, at 4.51 minutes there is 1 minutes hold at 20% B to re-equilibrate the column. The column was run at 40° C. by adjusting the column oven to 40° C. The flow rate was 1.5 ml per minute. The injection volume was 10 to 30 microliters.

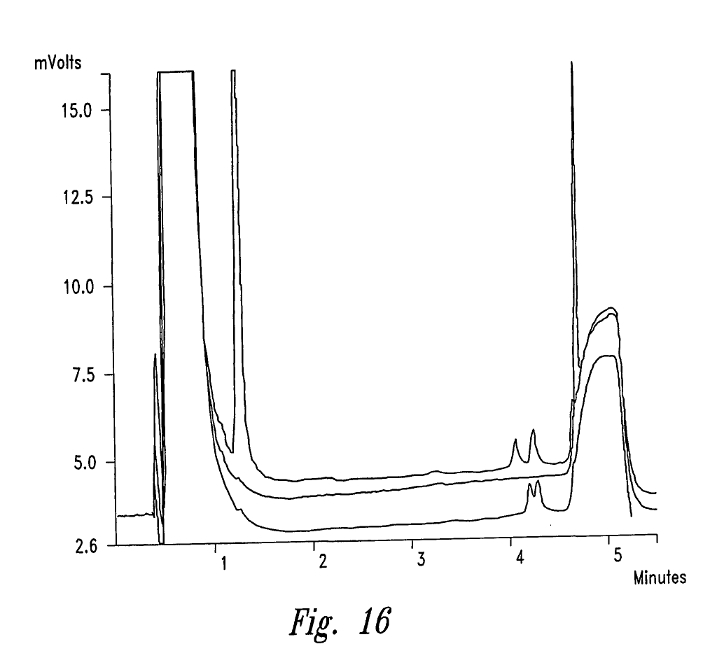

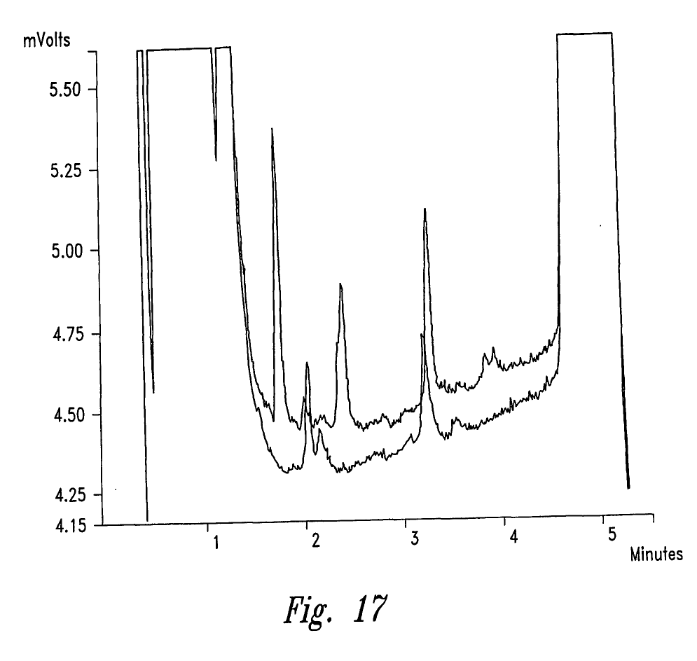

In FIG. 16, the controls for the chromatograms are shown. In Trace 1, the no-template control is shown in which the unincorporated primers (primer set 1) can be seen eluting after the 4 minute mark. In Trace 3, the no-template control is shown in which the unincorporated primers (primer set 2) can be seen eluting after the 4 minute mark. In trace 2, the +template control is seen prior to cutting with Fok I. The large amplicon is seen eluting at 4.6 minutes. In Trace 1, a peak is seen at 1.24 minutes which is due to the Fok I enzyme of buffer (the no-template control was mixed with the Fok I buffer components as a control). The large peak at 0.5 to 1 minute is due to the PCR components. In FIG. 17 is shown the short fragments generated by the Fok I enzyme double digest. 6-mers and 8-mers were expected. For primer set 1, peaks are seen at 2.0 and 3.2 minutes and for primers set 2, peaks are seen at 2.4 and 3.3 minutes. Therefore, SNP fragments can be easily generated and detected by the double-Fok I amplification and cutting.

### Example 5

### GENOTYPE ASSIGNMENT USING FLUORESCENCE POLARIZATION-Eco NI ASSAY

This example discloses the use of fluorescence polarization (FP) and the EcoN I template-directed primer extension (fill-in) assay in assigning genotype.

Enzymes

EcoN I was obtained from New England BioLabs (Beverly Mass.), AmpliTaq and AmpliTaq-FS DNA polymerase were obtained from Perkin-Elmer Applied Biosystems Division (Foster City, Calif.).

Oligonucleotides

Oligonucleotides used are listed in Table 7. Four synthetic 48-mers with identical sequence except for position 23 were prepared (CF508-48), the variant bases are shown as boldface letters. PCR and EcoN I primers and synthetic template oligonucleotides were obtained from Life Technologies (Grand Island, N.Y.).

Dye-Labeled Dideoxyribonucleoside Triphosphates

Dideoxyribonucleoside triphosphates labeled with FAM, ROX, TMR, BFL, and BTR were obtained from NEN Life Science Products, Inc. (Boston, Mass.). Unlabeled ddNTPs were purchased from Pharmacia Biotech (Piscataway, N.J.).

PCR Amplification

Human genomic DNA (20 ng) from 34 unrelated individuals and 6 negative controls (water-blanks) were amplified for the marker D18S8 in 20-μl reaction mixtures containing 10 mM Tris-HCl (pH 8.3), 50 mM KCl, 1.5 mM MgCl₂, 0.2 mM dNTP, 1 μM of each primer, and AmpliTaq DNA polymerase (1 unit). The reaction mixture was held at 94° C. for 2 min followed by 10 cycles of 94° C. for 10 sec, ramping to 60° C. >90 sec, held at 60° C. for 30 sec, followed by 30 cycles of 94° C. for 10 sec, and 53° C. for 30 sec. For hemochromatosis mutation C282Y, 42 samples and 6 negative controls were amplified in the same buffer with these cycling conditions: 94° C. for 2 min followed by 10 cycles of 94° C. for 10 sec, ramping to 68° C. >90 sec, held at 68° C. for 30 sec, followed by 30 cycles of 94° C. for 10 sec, and 62° C. for 30 sec. At the end of the reaction, the reaction mixtures were held at 4° C. until further use.

Primer and dNTP Degradation

At the end of the PCR assay, 10 μl of an enzymatic cocktail containing shrimp alkaline phosphatase (2 units), E. coli exonuclease I (1 unit) in shrimp alkaline phosphatase buffer [20 mM Tris-HCl (pH 8.0), 10 mM MgCl₂] was added to the PCR product. The mixture was incubated at 37° C. for 30 min before the enzymes were heat inactivated at 95° C. for 15 min. The DNA mixture was kept at 4° C. and used in the FP-EcoN I assay without further quantification or characterization.

Primer Extension

To the PCR product was added 10 μl of 10× EcoN I buffer reaction mixture containing the EcoN I buffer and enzyme [100 mM Tris-HCl (pH 7.5), 50 mM NaCl, 10 mM MgCl₂, 0.025% Triton X-100, and 4 units of enzyme per genotype], 25 nM of each allele-specific dye-labeled ddNTP, 100 nM unlabeled other two ddNTPs, and AmpliTaq DNA polymerase FS (1 unit). The reaction mixtures were incubated at 93° 1 min, followed by 35 cycles of 93° 10 sec and 55° 30 sec. At the end of the reaction, the samples were held at 4° C.

Fluorescence Polarization Measurement

After the primer extension reaction, 100 μl of EcoN I buffer and 50 μl of methanol were added to each tube before they were transferred to a microtiter plate for FP measurement on a Fluorolite FPM2 instrument (Jolley Consulting and Research, Grayslake, Ill.) or Analyst fluorescence reader (LJL Biosystems, Sunnyvale, Calif.). Fluorescence polarization value was calculated using the formula:

P=[Ivv−Ivh]/[Ivv+Ivh]

where Ivv is the emission intensity measured when the excitation and emission polarizers are parallel and Ivh is the emission intensity measured when the emission and excitation polarizers are oriented perpendicular to each other. The degree of polarization is expressed by the unit mP, or a 0.001 ratio between (Ivv−Ivh) and (Ivv+Ivh).

Genotype Assignment

The average FP value and standard deviation of the negative control samples were determined for each set of experiment. The FP value of the test sample reactions was then compared to the average FP value of the control samples. If the net change is >40 mP (more than seven times the standard deviation of the controls), the test sample is scored as positive for the allele.

Although dye-labeled dideoxy-terminators have been used extensively in sequencing reactions and the sensitivity and specificity of template-directed primer extension genotyping methods are well established, the use of FP as a detection method in a fill-in reaction has not been reported before this work was done. Three sets of experiments were performed to show that FP is a simple, highly sensitive and specific detection method in a homogeneous fill-in reaction for measuring SNPs, deletions and insertions. In the first set of experiments, four synthetic oligonucleotide templates containing the four possible nucleotides at one particular site in the middle of otherwise identical sequence were used to establish the sensitivity and specificity of FP detection of labeled nucleoside incorporation. In the second set of experiments, several dyes were tested for their utility in the fill-in assay. In the third set of experiments, PCR products were used as templates in a dual-color FP assay to show that accurate genotyping data could be obtained for both alleles of a marker or mutation in a homogeneous assay.

TABLE 7
Synthetic Templates and Primers Used in the FP Studies

| Oligo- | | |
|---|---|---|
| nucleotides | Sequence (5′to 3′) | |
| Synthetic | | |
| templates | | |
| CF508-48A | ATATTCATCATAGGAAACCTCAAAGAGGATATTTTCTTTAATGGTGCC | (SEQ ID NO.20) |
| CF508-48C | ATATTCATCATAGGAAACCTCACAGAGGATATTTTCTTTAATGGTGCC | (SEQ ID NO.21) |
| CF508-48G | ATATTCATCATAGGAAACCTCAGAGAGGATATTTTCTTTAATGGTGCC | (SEQ ID NO.22) |
| CF508-48T | ATATTCATCATAGGAAACCTCATAGAGGATATTTTCTTTAATGGTGCC | (SEQ ID NO.23) |
| PCR primers | | |
| C282Y-p1 | TGGCAAGGGTAAACAGATCC | (SEQ ID NO.24) |
| C282Y-p2 | CTCAGGCACTCCTCTCAACC | (SEQ ID NO.25) |
| D18S8-p1 | TTGCACCATGCTGAAGATTGT | (SEQ ID NO.26) |
| D18S8-p2 | ACCCTCCCCCTGATGACTTA | (SEQ ID NO.27) |

Synthetic template sequences (bold) are bases to be tested.

For each synthetic template (Table 7), one of the four possible bases was found at position 23. The synthetic 48-mers served as template in four separate reactions where each was incubated with EcoN I and one of the four 5-carboxy-fluorescein (FAM)-labeled terminators in the presence of AmpliTaq DNA polymerase FS. At the end of the EcoN I reaction, the reaction mixture was diluted and the fluorescence polarization was measured. Table 8 shows the results of these experiments.

TABLE 8
FP-TDI Assay with Synthetic Templates Using FAM-Labeled Dye Terminators

| Templates | FAM-ddA (mP)^{a} | FAM-ddC (mP)^{a} | FAM-ddG (mP)⁸ | FAM-ddU (mP)³ |
|---|---|---|---|---|
| CF508-48A | 52 | 36 | 54 | 89 |
| | 55 | 37 | 41 | 92 |
| | 52 | 39 | 48 | 101 |
| | 50 | 39 | 40 | 93 |
| CF508-48C | 57 | 37 | 121 | 39 |
| | 50 | 37 | 126 | 30 |
| | 55 | 39 | 115 | 40 |
| | 52 | 34 | 117 | 40 |
| CF508-48G | 52 | 92 | 42 | 42 |
| | 63 | 85 | 35 | 32 |
| | 50 | 91 | 40 | 47 |
| | 49 | 103 | 37 | 35 |
| CF508-48T | 186 | 32 | 48 | 34 |
| | 180 | 38 | 63 | 41 |
| | 183 | 36 | 43 | 33 |
| | 179 | 33 | 55 | 45 |
| Avg. ctrl. | 53 | 36 | 46 | 38 |
| S.D. Ctrl. | 4.0 | 2.5 | 8.3 | 5.3 |
| Avg. net chg.^{b} | 129 | 57 | 74 | 55 |

Positive reactions are in boldface type.
^{a}FP measurements for FAM were made with excitation at 485 nm and monitored at 530 nm.
^{b}Net change over average of control.

In all cases, only the nucleoside complementary to the polymorphic base was incorporated and showed significant FP change, with net gains of FP of at least 50 mP, which is nine times standard deviation of the controls.

### FP-EcoN I Assay With Different Terminators Labeled With Different Dyes

To identify different dyes suitable for multicolor detection in the same reaction, a number of different dyes were studied for their FP properties in the FP-EcoN I assay. With all the combinations of dye-terminators tested, the optimal set of terminators, chosen for minimal standard deviations in the control samples and large net changes in the positive samples, were found to be BODIPY-fluorscein-ddA (BFL-ddA), N,N,N′,N′-tetramethyl-6-carboxyrhodamine (TMR-ddC) b-carboxy-x-rhodamine-ddG (ROX-ddg), and BODIPY-Texas Red-ddU (BTR-ddU) (see Table 9). In all of these cases, the net increase in FP exceeded 10 times standard deviation of the mean of the control samples. In addition BFL-ddC, BFL-ddT, ROX-ddA, BTR-ddC, TMR-ddU, and all FAM terminators also worked.

TABLE 9
FP-EcoN I Assay with Synthetic Templates Using Different Dye Terminators

| Templates | BFL-ddA (mP)^{a} | TMR-ddC (mP)^{a} | ROX-ddG (mP)^{a} | BTR-ddU (mP)^{a} |
|---|---|---|---|---|
| CF508-48A | 38 | 43 | 77 | 174 |
| | 37 | 53 | 73 | 175 |
| | 31 | 36 | 78 | 174 |
| | 35 | 49 | 82 | 170 |
| CF508-48C | 20 | 50 | 214 | 32 |
| | 19 | 37 | 209 | 27 |
| | 20 | 56 | 215 | 25 |
| | 14 | 38 | 207 | 26 |
| CF508-48G | 23 | 247 | 84 | 23 |
| | 24 | 266 | 80 | 30 |
| | 22 | 253 | 75 | 23 |
| | 15 | 262 | 74 | 21 |
| CF508-48T | 113 | 52 | 81 | 32 |
| | 106 | 41 | 68 | 39 |
| | 108 | 59 | 81 | 30 |
| | 103 | 32 | 76 | 28 |
| Avg. ctrl. | 25 | 46 | 86 | 28 |
| S.D. Ctrl. | 8.2 | 8.8 | 4.6 | 5.0 |
| Avg. net chg.^{b} | 83 | 211 | 134 | 145 |

Positive reactions are in boldface type.
^{a}FP measurements for BFL were made with excitation at 480 nm and emission at 520 nm; for TMR the excitation was 535 nm and emission was 590 nm; for BTR the excitation was 591 nm and emission was 635 nm; for ROX the excitation was 580 nm and emission was 620 nm.
^{b}Net change over average of control.

### Dual Color FP-EcoN I Assay For Amplified Genomic DNA

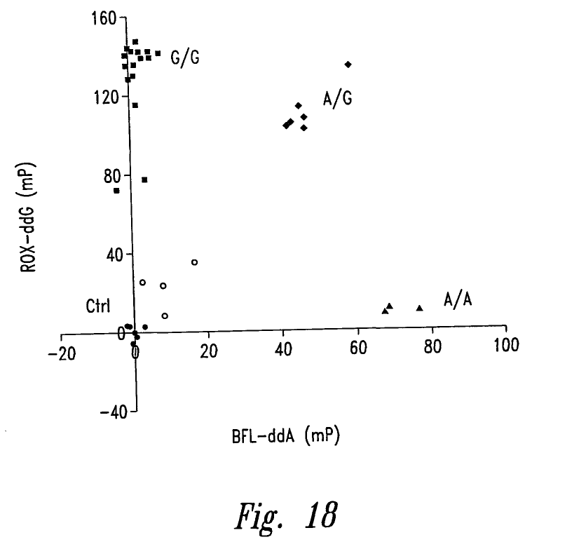

Markers D18S8 and the C282Y mutation in the human hereditary hemochromatosis (HFE) gene implicated in hemochromatosis were used in FP-TDI assays designed to test for both alleles in the same reaction. For marker D18S8, genomic DNA samples from 34 individuals were amplified and then cut with EcoN I and the 3′-ends filled in, in the presence of BFL-ddA and ROX-ddG. The FP values of the reaction mixtures were read at the BFL and ROX emission wavelengths, respectively, and the results are plotted and shown in FIG. 18 as changes in fluorescence polarization. The results are plotted in mP units above the average polarization of the negative controls. A change of 40 mP for a dye-terminator is scored as positive. DNA samples from 34 individuals and 6 water blanks were used. (▪) Samples positive for the G allele but negative for the A allele (homozygous G); (▴) samples positive for the A allele but negative for the G allele (homozygous A); (♦) samples positive for both alleles (heterozygotes); ( ) negative controls; (◯) samples with failed PCR amplification.

The FP values cluster into four groups. In the upper left corner of the plot, the samples have high FP for ROX-ddG but low FP for BFL-ddA, signifying that they are of homozygous G genotype (▪). The heterozygous A/G samples (♦) exhibit high FP values in both BFL-ddA and ROX-ddG and occupy the right upper corner of the plot. The homozygous A/A samples (▴) are found in the lower right corner, with low ROX-ddG but high BFL-ddA FP values. The negative controls (●) and samples with failed PCR reactions (o) occupy the area near the origin with low FP values for both dyes. The positive samples in both the BFL-ddA and the ROX reactions gave FP values that were >40 mP and 100 mP above average of controls, respectively. These values were >20 times standard deviation of the controls and the genotypes of the samples were easily assigned. Of 34 test samples, 4 gave inconclusive results because of PCR failure, which would prevent analysis by any method, including those based on gel electrophoresis.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for identifying one or more nucleotide(s) at a defined position in a single-stranded target nucleic acid, comprising
(a) providing a first oligonucleotide primer (ODNP) immobilized to a substrate, wherein the first ODNP comprises a nucleotide sequence complementary to a nucleotide sequence of the target nucleic acid at a location 3′ to the defined position, and further comprises a first constant recognition sequence (CRS) of a first strand of an interrupted restriction endonuclease recognition sequence (IRERS), but not a complete IRERS, the complete IRERS being a double-stranded oligonucleotide having the first strand and a second strand and comprising the first and a second CRS linked by a variable recognition sequence (VRS);
(b) exposing the immobilized first ODNP to the target nucleic acid and a second ODNP, wherein the second ODNP comprises a nucleotide sequence complementary to a nucleotide sequence of the complement of the target nucleic acid at a location 3′ to the defined position of the target nucleic acid, and further comprises the second CRS of the second strand of the IRERS;
(c) extending the first and second ODNPs so as to form a fragment having the complete IRERS wherein the nucleotide to be identified is within the VRS of the complete IRERS;
(d) cleaving the fragment with a restriction endonuclease that recognizes the complete IRERS; and
(e) characterizing a product of step (d) to thereby determine the identity of the nucleotide to be identified.

2. The method of claim 1 wherein the defined position is polymorphic.

3. The method of claim 1 wherein a mutation at the defined position is associated with a disease.

4. The method of claim 3 wherein the disease is selected from the group consisting of bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

5. The method of claim 1 wherein a mutation at the defined position is associated with drug resistance of a pathogenic microorganism.

6. The method of claim 1 wherein the single-stranded target nucleic acid is one strand of a denatured double-stranded nucleic acid.

7. The method of claim 6 wherein the double-stranded nucleic acid is genomic nucleic acid.

8. The method of claim 6 wherein the double-stranded nucleic acid is cDNA.

9. The method of claim 1 wherein the single-stranded target nucleic acid is derived from the genome of a pathogenic virus.

10. The method of claim 1 wherein the single-stranded target nucleic acid is derived from the genome or episome of a pathogenic bacterium.

11. The method of claim 1 wherein the target nucleic acid is synthetic nucleic acid.

12. The method of claim 1 wherein the substrate comprises a material selected from the group consisting of silicon, glass, paper, ceramic, metal, metalloid, and plastics.

13. The method of claim 1 wherein the first ODNP is non-covalently immobilized to the substrate.

14. The method of claim 1 wherein the first ODNP has 3′ and 5′ termini and is covalently immobilized to the substrate at the 5′ terminus.

15. The method of claim 1 wherein the first ODNP is prepared by photolithography.

16. The method of claim 1 wherein the first ODNP is synthesized on the substrate.

17. The method of claim 1 wherein the first ODNP is first synthesized and subsequently immobilized onto the substrate.

18. The method of claim 1 wherein the nucleotide sequence of the first ODNP that is complementary to the nucleotide sequence of the target nucleic acid is at least 12 nucleotides in length.

19. The method of claim 1 wherein the nucleotide sequence complementary to the nucleotide sequence of the complement of the target nucleic acid in the second ODNP is at least 12 nucleotides in length.

20. The method of claim 1 wherein step (c) comprises performing a polymerase chain reaction.

21. The method of claim 1 wherein step (d) produces a fragment with a 5′ overhang.

22. The method of claim 1 wherein step (d) produces a fragment with a blunt end.

23. The method of claim 1 wherein step (d) produces a fragment with a 3′ overhang.

24. The method of claim 1 wherein the restriction endonuclease is EcoN I.

25. The method of claim 21 wherein the nucleotide to be identified or the complement thereof is within the 5′ overhang.

26. The method of claim 25 wherein step (e) further comprises filling a 3′ recessed terminus corresponding to the 5′ overhang with one or more nucleoside triphosphates.

27. The method of claim 26 wherein step (e) further comprises washing the substrate before filling the 3′ recessed terminus.

28. The method of claim 26 wherein the nucleoside triphosphate comprises a detectable label.

29. The method of claim 28 wherein the detectable label is selected from the group consisting of a fluorophore and a radioisotope.

30. The method of claim 1 wherein the product of step (c) characterized in step (e) is not immobilized to the substrate.

31. The method of claim 1 wherein the product of step (c) characterized in step (e) is immobilized to the substrate.

32. The method of claim 1 wherein step (e) is performed at least partially by the use of a technique selected from the group consisting of mass spectrometry, liquid chromatography, fluorescence polarization, electron ionization, gel electrophoresis, and capillary electrophoresis.

33. An immoblilized oligonucleotide primer (ODNP), comprising
(a) an oligonucleotide sequence complementary to a nucleotide sequence of a single-stranded target nucleic acid at a location 3′ to a defined position, the oligonucleotide sequence having 3′ and 5′ termini; and
(b) at a location 3′ to the oligonucleotide sequence of (a), a first constant recognition sequence (CRS) of a first strand of an interrupted restriction endonuclease recognition sequence (IRERS), but not a complete IRERS, the complete IRERS being a double-stranded oligonucleotide having the first strand and a second strand and comprising the first CRS and a second CRS linked by a variable recognition sequence (VRS).

34. The immobilized ODNP of claim 33 wherein the oligonucleotide sequence of (a) is at least 18 nucleotides in length.

35. The immobilized ODNP of claim 33 further comprising one or more nucleotides complementary to the target nucleic acid at a location 3′ to the first CRS.

36. The immobilized ODNP of claim 33 wherein the ODNP is non-covalently immobilized to the substrate.

37. The immobilized ODNP of claim 33 wherein the ODNP has 3′ and 5′ termini and is covalently immobilized to the substrate at the 5′ terminus.

38. The immobilized ODNP of claim 33 wherein the ODNP is 15-80 nucleotides in length.

39. The immobilized ODNP of claim 33 wherein the complete IRERS is recognizable by EcoN I.

40. The immobilized ODNP of claim 33 wherein the defined position in the target nucleic acid is polymorphic.

41. The immobilized ODNP of claim 33 wherein a mutation at the defined position in the target nucleic acid is associated with a disease.

42. An immobilized oligonucleotide primer (ODNP) having regions A, B, C, D, E and F, the ODNP being partially complementary to a target nucleic acid as shown below: 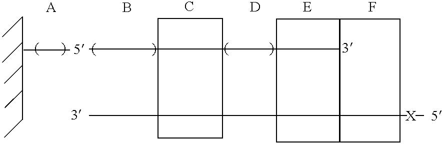
A designates an optional linking element that links the 5′ end of the ODNP to a solid support;
B designates an optional nucleotide sequence;
C designates a nucleotide sequence that is complementary to a nucleotide sequence of a single-stranded target nucleic acid at a location 3′ to a defined position “X” of the target nucleic acid;
D designates a first constant recognition sequence (CRS) of a first strand of an interrupted restriction endonuclease recognition sequence (IRERS), but not a complete IRERS, the complete IRERS being a double-stranded oligonucleotide having the first strand and a second strand and comprising the first CRS and a second CRS linked by a variable recognition sequence (VRS) having a number n of variable nucleotides;
E designates an optional nucleotide sequence; and
F designates an optional gap of nucleotides, where the number of nucleotides within regions E and F is within the range 0 to n-1.

43. The primer of claim 42 wherein A is absent.

44. The primer of claim 42 wherein A is present.

45. The primer of claim 44 wherein A is selected from a polyether and a polyester.

46. The primer of claim 44 where A is cleavable.

47. The primer of claim 42 wherein B comprises 1 to 50 nucleotides.

48. The primer of claim 42 wherein C comprises 2-30 nucleotides.

49. The primer of claim 42 wherein D comprises 2-6 nucleotides.

50. The primer of claim 42 wherein D has the sequence 5′-CCT-3′.

51. The primer of claim 42 wherein E is absent.

52. The primer of claim 42 wherein E is present.

53. The primer of claim 52 wherein E comprises 1-8 nucleotides.

54. The primer of claim 53 wherein E is complementary to the target nucleic acid.

55. The primer of claim 42 wherein F is absent.

56. The primer of claim 42 wherein F is present.

57. The primer of claim 56 wherein F comprises 1-8 nucleotides.

58. The primer of claim 42 wherein the number of nucleotides with regions B, C, D and E is between 15-80 nucleotides.

59. The primer of claim 42 wherein the immobilization is non-covalent attachment to the solid support.

60. The primer of claim 42 wherein the immobilization is covalent attachment to the solid support.

61. A composition comprising an immobilized oligonucleotide primer (ODNP) and a target nucleic acid, the ODNP having regions A, B, C, D, E and F and being partially complementary to the target nucleic acid, as shown below: 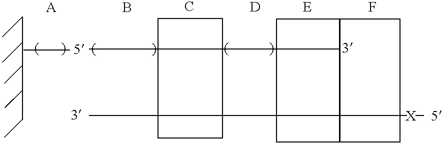
A designates an optional linking element that links the 5′ end of the ODNP to a solid support;
B designates an optional nucleotide sequence;
C designates a nucleotide sequence that is complementary to a nucleotide sequence of a single-stranded target nucleic acid at a location 3′ to a defined position “X” of the target nucleic acid;
D designates a first constant recognition sequence (CRS) of a first strand of an interrupted restriction endonuclease recognition sequence (IRERS), but not a complete IRERS, the complete IRERS being a double-stranded oligonucleotide having the first strand and a second strand and comprising the first CRS and a second CRS linked by a variable recognition sequence (VRS) having a number N of variable nucleotides;
E designates an optional nucleotide sequence; and
F designates an optional gap of nucleotides, where the number of nucleotides within regions E and F is within the range 0 to N-1.

62. The composition of claim 61 wherein A is absent.

63. The composition of claim 61 wherein A is present.

64. The composition of claim 63 wherein A is selected from a polyether and a polyester.

65. The composition of claim 63 wherein A is cleavable.

66. The composition of claim 61 wherein B comprises 1 to 50 nucleotides.

67. The composition of claim 61 wherein C comprises 2-30 nucleotides.

68. The composition of claim 61 wherein D comprises 2-6 nucleotides.

69. The composition of claim 61 wherein D has the sequence 5′-CCT-3′.

70. The composition of claim 61 wherein E is absent.

71. The composition of claim 62 wherein E is present.

72. The composition of claim 71 wherein E comprises 1-8 nucleotides.

73. The composition of claim 71 wherein E is complementary to the target nucleic acid.

74. The composition of claim 61 wherein F is absent.

75. The composition of claim 61 wherein F is present.

76. The composition of claim 75 wherein F comprises 1-8 nucleotides.

77. The composition of claim 61 wherein the number of nucleotides within regions B, C, D and E is between 15-80 nucleotides.

78. The composition of claim 61 wherein the immobilization is non-covalent attachment to the solid support.

79. The composition of claim 61 wherein the immobilization is covalent attachment to the solid support.

80. The composition of claim 61 wherein X is a single nucleotide polymorphism (SNP).

81. The composition of claim 61 wherein X is polymorphic.

82. The composition of claim 61 wherein X is a mutation associated with a disease.

83. The composition of claim 82 wherein the disease is selected from bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

84. An array, comprising:
(a) a substrate having a plurality of distinct areas; and
(b) a plurality of oligonucleotide primers (ODNPs) immobilized to the distinct areas wherein an ODNP in the plurality comprises
(i) an oligonucleotide sequence complementary to a nucleotide sequence of a single-stranded target nucleic acid at a location 3′ to a defined position at which position a nucleotide is to be identified, the oligonucleotide sequence having 3′ and 5′ termini, and
(ii) at the 3′ terminus of the oligonucleotide sequence of (i), a first constant recognition sequence (CRS) of a first strand of an interrupted restriction endonuclease recognition sequence (IRERS), but not a complete IRERS, the complete IRERS being a double-stranded nucleic acid having the first strand and a second strand and comprising the first CRS and a second CRS linked by a variable recognition sequence (VRS).

85. The array of claim 84 wherein the ODNPs in any one of the distinct areas are homogeneous, but different from the ODNPs in a second distinct area.

86. The array of claim 84 wherein the ODNPs in at least one of the distinct areas are heterogeneous.

87. The array of claim 84, wherein an ODNP is non-covalently immobilized to the substrate.

88. The array of claim 84, wherein an ODNP has 3′ and 5′ termini and is covalently immobilized to the substrate at the 5′ terminus.

89. The array of claim 84, wherein the plurality of ODNPs are prepared by photolithography.

90. The array of claim 84, wherein the plurality of ODNPs are synthesized on the substrate.

91. The array of claim 84, wherein the plurality of ODNPs are first synthesized and subsequently immobilized to the substrate.

92. The array of claim 84 wherein each ODNP is 15-80 nucleotides in length.

93. The array of claim 84 wherein for each ODNP, the oligonucleotide sequence of (i) is at least 12 nucleotides in length.

94. The array of claim 84 wherein at least one of the plurality of ODNPs further comprises one or more nucleotides complementary to the target nucleic acid at a location 3′ to the first CRS.

95. The array of claim 84 wherein the defined position is polymorphic.

96. The array of claim 84 wherein a mutation at the defined position is associated with a disease.

97. The array of claim 84, wherein the complete IRERS is recognizable by EcoN I.

98. The array of claim 84 wherein 1000 to 10¹² ODNP molecules are immobilized in at least one in the plurality of distinct areas.

99. The array of claim 84 wherein the substrate has 10-100 distinct areas.

100. The array of claim 84 wherein the substrate has 101-400 distinct areas.

101. The array of claim 84 wherein the substrate has 401-1000 distinct areas.

102. The array of claim 84 wherein the substrate has more than 1000 distinct areas.

103. The array of claim 84 wherein the substrate is made of a material selected from the group consisting of silicon, glass, paper, ceramic, metal, metalloid, and plastic.

104. The array of claim 84 wherein the single-stranded target nucleic acid is one strand of a denatured double-stranded nucleic acid.

105. The array of claim 104 wherein the double-stranded nucleic acid is genomic DNA.

106. The array of claim 84 wherein the target nucleic acids complementary to the ODNP(s) that comprise sequences (i) and (ii) are from one organism.

107. The array of claim 84 wherein the target nucleic acids complementary to the ODNP(s) that comprise sequences (i) and (ii) are from two or more organisms of one species.

108. The array of claim 84 wherein the ODNP(s) in any one of the distinct areas are the same as the ODNP(s) in a second distinct area.

109. The array of claim 84 wherein the surface of the array has raised portions to delineate the distinct areas.

110. A method, comprising
(a) providing a first set of oligonucleotide primers (ODNPs) immobilized to a substrate in a plurality of distinct areas wherein each ODNP of the first set comprises
(i) an oligonucleotide sequence complementary to a nucleotide sequence of a single-stranded target nucleic acid at a location 3′ to a defined position whereat a nucleotide is to be identified, and
(ii) a first constant recognition sequence (CRS) of a first strand of an interrupted restriction endonuclease recognition sequence (IRERS), but not a complete IRERS, the complete IRERS being a double-stranded nucleic acid having the first strand and a second strand and comprising the first CRS and a second CRS linked by a variable recognition sequence (VRS);
(b) exposing the immobilized first set of ODNPs to one or more target nucleic acids and a second set of ODNPs wherein each ODNP of the second set comprises
(i) an oligonucleotide sequence complementary to a nucleotide sequence of the complement of the single-stranded target nucleic acid at a location 3′ to the defined position, and
(ii) the second CRS of the second strand of the complete IRERS;
(c) extending the ODNPs of the first and second sets so as to form one or more fragments having the complete IRERS wherein the nucleotide(s) to be identified is within the VRS of the complete IRERS;
(d) cleaving the fragment(s) with a restriction endonuclease that recognizes the complete IRERS; and
(e) characterizing a product of step (d) to thereby determine the identity of the nucleotide to be identified.

111. The method of claim 110 wherein the ODNPs of the first set in any one of the distinct areas are homogeneous, but different from the ODNPs in a second distinct area.

112. The method of claim 110 wherein the ODNPs of the first set in at least one of the distinct areas are heterogeneous.

113. The method of claim 110 wherein each ODNP of the first set is non-covalently immobilized to the substrate.

114. The method of claim 110 wherein each ODNP of the first set has 3′ and 5′ termini and is covalently immobilized to the substrate at the 5′ terminus.

115. The method of claim 110 wherein the first set of ODNPs are immobilized to the substrate by photolithography.

116. The method of claim 110 wherein the first set of ODNPs are synthesized on the substrate.

117. The method of claim 110 wherein the first set of ODNPs are first synthesized and subsequently immobilized to the substrate.

118. The method of claim 110 wherein each of the first set of ODNPs is 15-80 nucleotides in length.

119. The method of claim 110 wherein each of the second set of ODNPs is 15-80 nucleotides in length.

120. The method of claim 110 wherein for each of the first set of ODNPs, the oligonucleotide sequence of (i) is at least 12 nucleotides in length.

121. The method of claim 110 wherein for each of the second set of ODNPs, the oligonucleotide sequence of (i) is at least 12 nucleotides in length.

122. The method of claim 110 wherein at least one of the first set of ODNPs further comprises one or more nucleotides complementary to the target nucleic acid at a location 3′ to the first CRS.

123. The method of claim 110 wherein at least one of the second set of ODNPs further comprises one or more nucleotides complementary to the complement of the target nucleic acid at a location 3′ to the second CRS.

124. The method of claim 110 wherein the defined position is polymorphic.

125. The method of claim 110 wherein a mutation at the defined position is associated with a disease.

126. The method of claim 110 wherein the disease is selected from the group consisting of bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

127. The method of claim 110 wherein 1000 to 10¹² ODNP molecules of the first set are immobilized in at least one of the plurality of distinct areas.

128. The method of claim 110 wherein the substrate has 10-100 distinct areas.

129. The method of claim 110 wherein the substrate has 101-400 distinct areas.

130. The method of claim 110 wherein the substrate has at least 401-1000 distinct areas.

131. The method of claim 110 wherein the substrate has more than 1000 distinct areas.

132. The method of claim 110 wherein the substrate is made of a material selected from the group consisting of silicon, glass, paper, ceramic, metal, metalloid, plastics and plastic copolymers.

133. The method of claim 110 wherein the single-stranded target nucleic acid is one strand of a denatured double-stranded nucleic acid.

134. The method of claim 133 wherein the double-stranded nucleic acid is genomic DNA.

135. The method of claim 133 wherein the double-stranded nucleic acid is cDNA.

136. The method of claim 110 wherein the target nucleic acid is synthetic nucleic acid.

137. The method of claim 110 wherein the target nucleic acids complementary to the ODNPs of the first set are from one organism.

138. The method of claim 110 wherein the target nucleic acids complementary to the ODNP of the first set are from two or more organisms of one species.

139. The method of claim 110 wherein the ODNP(s) of the first set in any one of the distinct areas are the same as the ODNP(s) in a second distinct area.

140. The method of claim 110 wherein step (c) comprises performing a polymerase chain reaction.

141. The method of claim 110 wherein step (d) produces a fragment with a 5′ overhang.

142. The method of claim 110 wherein step (d) produces a fragment with a 3′ overhang.

143. The method of claim 110 wherein step (d) produces a fragment with a blunt end.

144. The method of claim 141 wherein the nucleotide to be identified or the complement thereof is within the 5′ overhang produced by step (d).

145. The method of claim 141 wherein step (e) further comprises filling a 3′ recessed terminus corresponding to the 5′ overhang with one or more nucleoside triphosphates.

146. The method of claim 145 wherein the 3′ recessed terminus is filled in with a RNA polymerase.

147. The method of claim 145 wherein step (e) further comprises washing the substrate before filling the 3′ recessed terminus.

148. The method of claim 145 wherein the nucleoside triphosphate comprises a detectable label.

149. The method of claim 148 wherein the detectable label is selected from the group consisting of a flurophore and a radioisotope.

150. The method of claim 110 wherein step (e) is performed at least partially by the use of a technique selected from the group consisting of mass spectrometry, liquid chromatography, fluorescence polarization, electron ionization, gel electrophoresis, and capillary electrophoresis.

151. The method of claim 110 wherein the restriction endonuclease is EcoN I.

152. The method of claim 110 wherein the product of step (c) characterized in step (e) is not immobilized to the substrate.

153. The method of claim 110 wherein the product of step (c) characterized in step (e) is immobilized to the substrate.

154. The method of claim 110 wherein the substrate has raised portion to delineate the distinct areas.

155. A method, comprising
(a) exposing the array of claim 84 to one or more target nucleic acids and a set of ODNPs wherein each ODNP of the set comprises
(i) an oligonucleotide sequence complementary to a nucleotide sequence of the complement of the single-stranded target nucleic acid at a location 3′ to the defined position, and
(ii) the second CRS of the second strand of the complete IRERS;
(b) extending the immobilized ODNPs of the array and the ODNPs of the set so as to form one or more fragments having the complete IRERS wherein the nucleotide(s) to be identified is within the VRS of the complete IRERS;
(c) cleaving the fragment(s) with a restriction endonuclease that recognizes the complete IRERS; and
(d) characterizing a product of step (d) to thereby determine the identity of the nucleotide to be identified.

156. A kit for genotyping comprising the array of claim 84.

157. A kit for genotyping comprising the array of any one of claims 85 to 108.

158. The kit of claim 156 further comprising a restriction endonuclease that recognizes the complete IRERS.

159. The kit of claim 158 further comprising a DNA polymerase.


SEQUENCE LISTING (28 sequences)

SEQ ID NO: 1 - DNA, 11 bp, unknown
  DNA motif cleaved by Bsl I restriction enzyme
ccnnnnnnngg

SEQ ID NO: 2 - DNA, 11 bp, UNKNOWN
  DNA motif cleaved by Bsl I restriction enzyme
ccnnnnnnngg

SEQ ID NO: 3 - DNA, 11 bp, UNKNOWN
  DNA motif cleaved by EcoN I restriction enzyme
cctnnnnnagg

SEQ ID NO: 4 - DNA, 11 bp, unknown
  DNA motif cleaved by EcoN I restriction enzyme
cctnnnnnagg

SEQ ID NO: 5 - DNA, 11 bp, unknown
  Recognition sequence of restriction enzyme Ahd I
gacnnnnngtc

SEQ ID NO: 6 - DNA, 11 bp, unknown
  Recognition sequence of restriction enzyme Bgl I
gccnnnnnggc

SEQ ID NO: 7 - DNA, 10 bp, unknown
  Recognition sequence of restriction enzyme Xmn I
gaannnnttc

SEQ ID NO: 8 - DNA, 10 bp, Artificial Sequence
  Single stranded oligonucleotide fragment
gaacatccat

SEQ ID NO: 9 - DNA, 24 bp, Artificial Sequence
  Primer for generating amplicons from target nucleic acids.
gaagtgatggggatgcggaaagag

SEQ ID NO: 10 - DNA, 24 bp, Artificial Sequence
  Primer for generating amplicons from target nucleic acids.
gtaagccacacatccaggaacggg

SEQ ID NO: 11 - DNA, 24 bp, Artificial Sequence
  Primer for generating amplicons from target nucleic acids.
aaagctggcaggatgaccggcaga

SEQ ID NO: 12 - DNA, 24 bp, Artificial Sequence
  Primer for generating amplicons from target nucleic acids.
agcgtctgttcatcctcgtggcgg

SEQ ID NO: 13 - DNA, 60 bp, Homo sapiens
gaagtgatggcagagcggaaagagcattattcagcgcccgttcctgaccgtgtggcttac

SEQ ID NO: 14 - DNA, 60 bp, Homo sapiens
gaaagctggctgattgaccggcagattattatgggccgccacgacgatgaacagacgctg

SEQ ID NO: 15 - DNA, 10 bp, Artificial Sequence
  Single stranded oligonucleotide fragment
attattcagc

SEQ ID NO: 16 - DNA, 10 bp, Artificial Sequence
  genotyping fragment
gaatatccat

SEQ ID NO: 17 - DNA, 10 bp, Artificial Sequence
  genotyping fragment
gaatatccac

SEQ ID NO: 18 - DNA, 24 bp, Artificial Sequence
  Primer for generating amplicons from target nucleic acids
gtaagccacaggatgaggaacggg

SEQ ID NO: 19 - DNA, 24 bp, Artificial Sequence
  Primer for generating amplicons from target nucleic acids
agcgtctgttggatgtcgtggcgg

SEQ ID NO: 20 - DNA, 48 bp, Artificial Sequence
  Synthetic template used in FP studies
atattcatcataggaaacctcaaagaggatattttctttaatggtgcc

SEQ ID NO: 21 - DNA, 48 bp, Artificial Sequence
  Synthetic template used in FP studies
atattcatcataggaaacctcacagaggatattttctttaatggtgcc

SEQ ID NO: 22 - DNA, 48 bp, Artificial Sequence
  Synthetic template used in FP studies
atattcatcataggaaacctcagagaggatattttctttaatggtgcc

SEQ ID NO: 23 - DNA, 48 bp, Artificial Sequence
  Synthetic template used in FP studies
atattcatcataggaaacctcatagaggatattttctttaatggtgcc

SEQ ID NO: 24 - DNA, 20 bp, Artificial Sequence
  PCR primer used in FP studies
tggcaagggtaaacagatcc

SEQ ID NO: 25 - DNA, 20 bp, Artificial Sequence
  PCR primer used in FP studies
ctcaggcactcctctcaacc

SEQ ID NO: 26 - DNA, 21 bp, Artificial Sequence
  PCR primer used in FP studies
ttgcaccatgctgaagattgt

SEQ ID NO: 27 - DNA, 20 bp, Artificial Sequence
  PCR primer used in FP studies
accctccccctgatgactta

SEQ ID NO: 28 - DNA, 10 bp, Homo sapiens
gaacatccac